SEQUENCE LISTING (1 sequence)

SEQ ID NO: 1 - protein, 8 aa, Artificial Sequence
  unknown origin
RVASKRAS

(19) United States
(12) Patent Application Publication
(10) Pub. No.: US 2010/0291034 A1
(43) Pub. Date: Nov. 18, 2010

(54) COMBINATIONS OF HCV PROTEASE INHIBITOR(S) AND CYP3A4 INHIBITOR(S), AND METHODS OF TREATMENT RELATED THERETO

(75) Inventors: Robert O RALSTON, II, Union, NJ (US); Julie M. Strizki, Yardley, PA (US); Jaromir Vlach, Annandale, NJ (US); Samir K. Gupta, East Brunswick, NJ (US); Edward M. O'Mara, JR., Skillman, NJ (US); Anima Ghosal, Edison, NJ (US); Michelle A. Treitel, New York, NY (US); James F. McLeod, Morris Township, NJ (US); Ronald E. White, Cranbury, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation, Kenilworth, NJ (US)

(21) Appl. No.: 12/839,097

(22) Filed: Jul. 19, 2010

Related U.S. Application Data
(63) Continuation of application No. 11/725,518, filed on Mar. 19, 2007, now abandoned.
(60) Provisional application No. 60/785,761, filed on Mar. 23, 2006.
(60) Provisional application No. 60/809,713, filed on May 31, 2006.

(51) Int. Cl. A61K 38/21; A61K 38/05; A61P 31/14

(52) U.S. Cl. 424/85.7; 514/4.3; 424/85.4

(57) ABSTRACT

Disclosed are medicaments, pharmaceutical compositions, pharmaceutical kits, and methods based on combinations comprising, separately or together: (a) a CYP3A4 inhibitor; and (b) a HCV protease inhibitor; for concurrent or consecutive administration in treating a human subject infected with HCV.

### CROSS-REFERENCE TO RELATED APPLICATIONS

This application is a continuation of U.S. patent application Ser. No. 11/725,518, filed Mar. 19, 2007, which in turn claims the benefit of priority to U.S. Provisional Patent Applications 60/785,761 filed Mar. 23, 2006, and 60/809,713 filed May 31, 2006, the entire disclosure of each of the priority applications is hereby incorporated by reference.

### FIELD OF THE INVENTION

The present invention relates to medicaments, pharmaceutical compositions, pharmaceutical kits, and methods based on combinations comprising, separately or together: (a) at least one cytochrome P450 isoenzyme 3A4 (CYP3A4) inhibitor; and (b) at least one hepatitis C virus (HCV) protease inhibitor; and optionally (c) at least one other therapeutic agent; for concurrent or consecutive administration in treating or ameliorating one or more symptoms of HCV or disorders associated with HCV in a subject in need thereof. The present invention also provides medicaments, pharmaceutical compositions, pharmaceutical kits, and methods based on combinations comprising, separately or together: (a) at least one cytochrome P450 isoenzyme 3A4 (CYP3A4) inhibitor; and (b) at least one anti-hepatitis C virus (anti-HCV) agent selected from the group consisting of a HCV protease inhibitor, a HCV polymerase inhibitor, a HCV NS3 helicase inhibitor, an inhibitor of HCV entry, an inhibitor of HCV p7, and a combination of two or more thereof; and optionally (c) at least one other therapeutic agent; for concurrent or consecutive administration in treating or ameliorating one or more symptoms of HCV or disorders associated with HCV in a subject in need thereof.

### BACKGROUND OF THE INVENTION

Citation of or reference to any application or publication in this Section or any Section of this application is not an admission that such document is available as prior art to the present invention.

HCV has been implicated in cirrhosis of the liver and in induction of hepatocellular carcinoma. The prognosis for patients suffering from HCV infection is currently poor. HCV infection is more difficult to treat than other forms of hepatitis due to the lack of immunity or remission associated with HCV infection. Current data indicates a less than 50% survival rate at four years post cirrhosis diagnosis. Patients diagnosed with localized resectable hepatocellular carcinoma have a five-year survival rate of 10-30%, whereas those with localized unresectable hepatocellular carcinoma have a five-year survival rate of less than 1%.

Current therapies for HCV include interferon-α (INF_{α}) and combination therapy with ribavirin and interferon. See, e.g., Berenguer and Wright, Proc Assoc Am Physicians, 110(2):98-112 (1998). These therapies suffer from a low sustained response rate and frequent side effects. See, e.g., Hoofnagle and di Bisceglie, N Engl J Med, 336(5):347-356 (1997). Currently, no vaccine is available for HCV infection.

HCV is a (+)-sense single-stranded RNA virus that has been implicated as the major causative agent in non-A, non-B hepatitis (NANBH), particularly in blood-associated NANBH (BB-NANBH) (see, International Patent Application Publication No. WO 89/04669 and European Patent Application Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliary cirrhosis.

Recently, a HCV protease necessary for polypeptide processing and viral replication has been identified, cloned and expressed; (see, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3, 4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. Other chymotrypsin-like enzymes are elastase, factor Xa, thrombin, trypsin, plasmin, urokinase, tPA and PSA. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating five viral proteins during viral replication. This has made the HCV NS3 serine protease an attractive target for antiviral chemotherapy.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e., trans).

Analysis of the natural cleavage sites for HCV protease revealed the presence of cysteine at P1 and serine at P1′ and that these residues are strictly conserved in the NS4a/NS4b, NS4b/NS5a and NS5a/NS5b junctions. The NS3/NS4a junction contains a threonine at P1 and a serine at P1′. The Cys→Thr substitution at NS3/NS4a is postulated to account for the requirement of cis rather than trans processing at this junction. See, e.g., Pizzi et al., Proc Natl Acad Sci (USA), 91(3):888-892 (1994), Failla et al., Fold Des, 1(1):35-42 (1996), Wang et al., J Virol, 78(2):700-709 (2004). The NS3/NS4a cleavage site is also more tolerant of mutagenesis than the other sites. See, e.g., Kolykhalov et al., J Virol, 68(11):7525-7533 (1994). It has also been found that acidic residues in the region upstream of the cleavage site are required for efficient cleavage. See, e.g., Komoda et al., J Virol, 68(11):7351-7357 (1994).

Inhibitors of HCV protease that have been reported include antioxidants (see, International Patent Application Publication No. WO 98/14181), certain peptides and peptide analogs (see, International Patent Application Publication No. WO 98/17679, Landro et al., Biochemistry, 36(31):9340-9348 (1997), Ingallinella et al., Biochemistry, 37(25):8906-8914 (1998), Llinàs-Brunet et al., Bioorg Med Chem Lett, 8(13):1713-1718 (1998)), inhibitors based on the 70-amino acid polypeptide eglin c (Martin et al., Biochemistry, 37(33):11459-11468 (1998), inhibitors affinity selected from human pancreatic secretory trypsin inhibitor (hPSTI-C3) and minibody repertoires (MBip) (Dimasi et al., J Virol, 71(10):7461-7469 (1997)), cV_{H}E2 (a “camelized” variable domain antibody fragment) (Martin et al., Protein Eng, 10(5):607-614 (1997), and α1-antichymotrypsin (ACT) (Elzouki et al., J Hepat, 27(1):42-48 (1997)). Reference is also made to the PCT Publications, No. WO 98/17679, published Apr. 30, 1998 (Vertex Pharmaceuticals Incorporated); WO 98/22496, published May 28, 1998 (F. Hoffmann-La Roche AG); and WO 99/07734, published Feb. 18, 1999 (Boehringer Ingelheim Canada Ltd.). A ribozyme designed to selectively destroy HCV RNA has recently been disclosed (see, Bio World Today, 9(217):4 (Nov. 10, 1998)).

The following pending and copending U.S. patent applications disclose various types of peptides and/or other compounds as NS-3 serine protease inhibitors of HCV: Ser. No. 60/194,607, filed Apr. 5, 2000 (corresponding to U.S. Publication No. 2002/010781), and Ser. No. 60/198,204, filed Apr. 19, 2000 (corresponding to U.S. Publication No. 2002/0016294), Ser. No. 60/220,110, filed Jul. 21, 2000 (corresponding to U.S. Publication No. 2002/0102235), Ser. No. 60/220,109, filed Jul. 21, 2000 (corresponding to U.S. Publication No. 2003/0036501), Ser. No. 60/220,107, filed Jul. 21, 2000 (corresponding to U.S. Publication No. 2002/0160962), Ser. No. 60/254,869, filed Dec. 12, 2000 (corresponding to U.S. Publication No. 2002/0147139), Ser. No. 60/220,101, filed Jul. 21, 2000 (corresponding to U.S. Publication No. 2002/0068702), Ser. No. 60/568,721 filed May 6, 2004 (corresponding to WO 2005/107745), and WO 2003/062265.

In drug metabolism, cytochrome P450 is probably the most important element of oxidative metabolism (also known as Phase I metabolism) in animals (metabolism in this context being the chemical modification or degradation of chemicals including drugs and endogenous compounds). Many drugs may increase or decrease the activity of various CYP isozymes in a phenomenon known as enzyme induction and inhibition. This is a major source of adverse drug interactions, since changes in CYP enzyme activity may affect the metabolism and clearance of various drugs. For example, if one drug inhibits the CYP-mediated metabolism of another drug, the second drug may accumulate within the body to toxic levels, possibly causing an overdose. Hence, these drug interactions may necessitate dosage adjustments or choosing drugs which do not interact with the CYP system. In addition, naturally occurring compounds may also cause a similar effect.

CYP3A4, in particular, is one of the most important enzymes involved in the metabolism of xenobiotics in the body. CYP3A4 is involved in the oxidation of the largest range of substrates of all the CYPs. CYP3A4 is also, correspondingly, present in the largest quantity of all the CYPs in the liver. In addition, although predominantly found in the liver, CYP3A4 is also present in other organs and tissues of the body where it may play an important role in metabolism. For example, CYP3A4 in the intestine plays an important role in the metabolism of certain drugs. Often the interaction of CYP3A4 allows prodrugs to be activated and absorbed—as in the case of the histamine H₁-receptor antagonist terfenadine. Notably, compounds found in grapefruit juice and some other fruit juices, including bergamottin, dihydroxybergamottin, and paradisin-A, have been found to inhibit CYP3A4-mediated metabolism of certain medications, leading to increased bioavailability and thus the strong possibility of overdosing.

Methods for improving the pharmacokinetics (e.g., increased half-life, increased time to peak plasma concentration, increased blood levels) of a HIV protease inhibitor which is metabolized by cytochrome P450 monooxygenase by coadministration with ritonavir (also known as ABT-538) an inhibitor of cytochrome P450 monooxygenase are described in U.S. Pat. No. 6,037,157 and U.S. Pat. No. 6,703,403.

There is a need for new treatments and therapies for HCV infection to treat, prevent or ameliorate of one or more symptoms of HCV, methods for modulating the activity of serine proteases, particularly the HCV NS3/NS4a serine protease, and for methods of modulating the processing of the HCV polypeptide.

Another aspect of the present invention is directed to inhibiting cathepsin activity. Cathepsins (Cats) belong to the papain superfamily of lysosomal cysteine proteases. Cathepsins are involved in the normal proteolysis and turnover of target proteins and tissues as well as in initiating proteolytic cascades by proenzyme activation and in participating in MHC class II molecule expression. Baldwin, Proc Natl Acad Sci, 90(14):6796-6800 (1993); Mizuochi, Immunol Lett, 43(3):189-193 (1994).

However, aberrant cathepsin expression has also been implicated in several serious human disease states. Cathepsins have been shown to be abundantly expressed in cancer cells, including breast, lung, prostate, glioblastoma and head/neck cancer cells, (Kos and Lah, Oncol Rep, 5(6):1349-1361 (1998); Yan et al., Biol Chem, 379(2):113-123 (1998); Mort and Buttle, Int J Biochem Cell Biol, 29(5): 715-720 (1997); Friedrich et al., Eur J Cancer, 35(1):138-144 (1999)) and are associated with poor treatment outcome of patients with breast cancer, lung cancer, brain tumor and head/neck cancer. Kos and Lah, supra. Additionally, aberrant expression of cathepsin is evident in several inflammatory disease states, including rheumatoid arthritis and osteoarthritis. Keyszer et al., Arthritis Rheum, 38(7):976-984 (1995).

The molecular mechanisms of cathepsin activity are not completely understood. Recently, it was shown that forced expression of cathepsin B rescued cells from serum deprivation-induced apoptotic death (Shibata et al., Biochem Biophys Res Commun, 251(1):199-203 (1998)) and that treatment of cells with antisense oligonucleotides of cathepsin B induced apoptosis. Isahara et al., Neuroscience, 91(1):233-249 (1999). These reports suggest an anti-apoptotic role for the cathepsins that is contrary to earlier reports that cathepsins are mediators of apoptosis. Roberts et al., Gastroenterology, 113(5):1714-1726 (1997); Jones et al., Am J Physiol, 275(4Pt1):G723-730 (1998).

Cathepsin K is a member of the family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard et al., J Biol Chem, 271(21):12517-12524 (1996); Drake et al., J Biol Chem, 271(21):12511-12516 (1996); Bromme et al., J Biol. Chem, 271(4):2126-2132 (1996).

Cathepsin K has been variously denoted as cathepsin O, cathepsin X or cathepsin O2 in the literature. The designation cathepsin K is considered to be the more appropriate one (name assigned by Nomenclature Committee of the International Union of Biochemistry and Molecular Biology).

Cathepsins of the papain superfamily of cysteine proteases function in the normal physiological process of protein degradation in animals, including humans, e.g., in the degradation of connective tissue. However, elevated levels of these enzymes in the body can result in pathological conditions leading to disease. Thus, cathepsins have been implicated in various disease states, including but not limited to, infections by pneumocystis carinii, trypsanoma cruzi, trypsanoma brucei, and Crithidia fusiculata; as well as in schistosomiasis malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa et al., Perspectives in Drug Discovery and Design, 2:445-458 (1994).

Cathepsin K is believed to play a causative role in diseases of excessive bone or cartilage loss. Bone is composed of a protein matrix in which spindle- or plate-shaped crystals of hydroxyapatite are incorporated. Type I Collagen represents the major structural protein of bone comprising approximately 90% of the structural protein. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodeling at discrete foci throughout life. These foci, or remodeling units, undergo a cycle consisting of a bone resorption phase followed by a phase of bone replacement. Bone resorption is carried out by osteoclasts, which are multinuclear cells of hematopoietic lineage. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

There are reports in the literature of the expression of Cathepsin B and L antigen and that activity is associated with early colorectal cancer progression. Troy et al., Eur J Cancer, 40(10):1610-1616 (2004). The findings suggest that cysteine proteases play an important role in colorectal cancer progression.

Cathepsin L has been shown to be an important protein mediating the malignancy of gliomas and it has been suggested that its inhibition may diminish their invasion and lead to increased tumor cell apoptosis by reducing apoptotic threshold. Levicar et al., Cancer Gene Ther, 10(2):141-151 (2003).

Katunuma et al., Arch Biochem Biophys, 397(2):305-311 (2002) reports on antihypercalcemic and antimetastatic effects of CLIK-148 in vivo, which is a specific inhibitor of cathepsin L. This reference also reports that CLIK-148 treatment reduced distant bone metastasis to the femur and tibia of melanoma A375 tumors implanted into the left ventricle of the heart.

Rousselet et al., Cancer Res, 64(1):146-151 (2004) reports that anti-cathepsin L single chain variable fragment (ScFv) could be used to inhibit the tumorigenic and metastatic phenotype of human melanoma, depending on procathepsin L secretion, and the possible use of anti-cathepsin L ScFv as a molecular tool in a therapeutic cellular approach.

Colella and Casey, Biotech Histochem, 78(2):101-108 (2003) reports that the cysteine proteinases cathepsin L and B participate in the invasive ability of the PC3 prostrate cancer cell line, and the potential of using cystein protease inhibitors such as cystatins as anti-metastatic agents.

Krueger et al., Cancer Gene Ther, 8(7):522-528 (2001) reports that in human osteosarcoma cell line MNNG/HOS, cathepsin L influences cellular malignancy by promoting migration and basement membrane degradation.

Frohlich et al., Arch Dermatol Res, 295(10):411-421 (2004) reports that cathepsins B and L are involved in invasion of basal cell carcinoma (BCC) cells. U.S. Provisional Patent Application Ser. No. 60/673,294, entitled “Compounds for Inhibiting Cathepsin Activity,” filed Apr. 20, 2005, (corresponding to U.S. Publication No. 2006/0252698), discloses various types of peptides and/or other compounds as inhibitors of cathepsin.

Cathepsins therefore are attractive targets for the discovery of novel chemotherapeutics and methods of treatment effective against a variety of diseases. There is a need for compounds and combinations useful in the inhibition of cathepsin activity and in the treatment of these disorders.

It would also be desirable to modify the pharmacokinetic behavior of HCV treatments and cathepsin inhibitors to enhance the efficacy and duration of action thereof.

### SUMMARY OF THE INVENTION

The present invention provides medicaments, pharmaceutical compositions, pharmaceutical kits, and methods based on combinations comprising, separately or together: (a) at least one CYP3A4 inhibitor; and (b) at least one HCV protease inhibitor; for concurrent or consecutive administration in treating or ameliorating one or more symptoms of HCV or disorders associated with HCV in a subject in need thereof.

In one embodiment, the present invention provides medicaments, pharmaceutical compositions, pharmaceutical kits, and methods based on combinations comprising, separately or together: (a) at least one cytochrome P450 isoenzyme 3A4 (CYP3A4) inhibitor; and (b) at least one hepatitis C virus (HCV) protease inhibitor which is a compound of Formula I to XXVI below or a pharmaceutically acceptable salt, solvate or ester thereof; with the proviso that when at least one CYP3A4 inhibitor is ritonavir, then at least one HCV protease inhibitor is not Formula Ia; for concurrent or consecutive administration in treating or ameliorating one or more symptoms of HCV or disorders associated with HCV in a subject in need thereof.

In one embodiment, the present invention provides medicaments and methods using the same comprising, separately or together: (a) at least one cytochrome P450 isoenzyme 3A4 (CYP3A4) inhibitor; and (b) at least one hepatitis C virus (HCV) protease inhibitor which is:

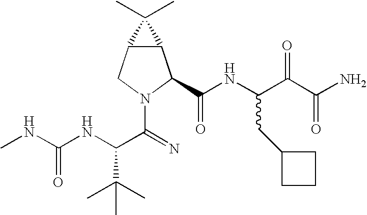

Formula Ia or a pharmaceutically acceptable salt, solvate or ester thereof; with the proviso that when at least one CYP3A4 inhibitor is ritonavir then at least one HCV protease inhibitor is not Formula Ia; for concurrent or consecutive administration in treating or ameliorating one or more symptoms of HCV or disorders associated with HCV in a subject in need thereof.

In a preferred embodiment, the present invention provides medicaments and methods using the same comprising, separately or together: (a) at least one cytochrome P450 isoenzyme 3A4 (CYP3A4) inhibitor; and (b) at least one hepatitis C virus (HCV) protease inhibitor which is:

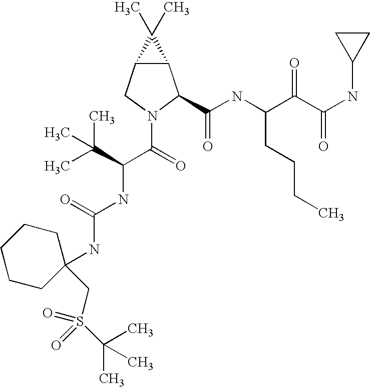

Formula XIVa or a pharmaceutically acceptable salt, solvate or ester thereof; for concurrent or consecutive administration in treating or ameliorating one or more symptoms of HCV or disorders associated with HCV in a subject in need thereof.

In another preferred embodiment, the present invention provides medicaments and methods using the same comprising, separately or together: (a) at least one cytochrome P450 isoenzyme 3A4 (CYP3A4) inhibitor; and (b) at least one hepatitis C virus (HCV) protease inhibitor which is:

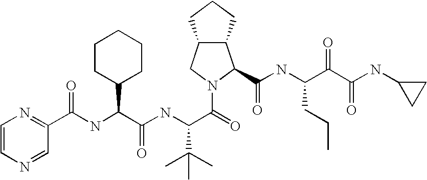

Formula XXVII or a pharmaceutically acceptable salt, solvate or ester thereof; for concurrent or consecutive administration in treating or ameliorating one or more symptoms of HCV or disorders associated with HCV in a subject in need thereof.

The present invention also provides medicaments, pharmaceutical compositions, pharmaceutical kits, and methods based on combinations comprising, separately or together: (a) at least one cytochrome P450 isozyme 3A4 (CYP3A4) inhibitor; and (b) at least one anti-HCV agent selected from the group consisting of a HCV protease inhibitor, a HCV polymerase inhibitor, a HCV NS3 helicase inhibitor, an inhibitor of HCV entry, an inhibitor of HCV p7, and a combination of two or more thereof; for concurrent or consecutive administration in treating or ameliorating one or more symptoms of HCV or disorders associated with HCV in a subject in need thereof.

In one embodiment, the present invention provides medicaments and methods using the same comprising, separately or together: (a) at least one cytochrome P450 isozyme 3A4 (CYP3A4) inhibitor; and (b) at least one anti-HCV agent which is a compound of Formula I to XXVI below or a pharmaceutically acceptable salt, solvate or ester thereof; with the proviso that when at least one CYP3A4 inhibitor is ritonavir then at least one anti-HCV agent is not Formula Ia; for concurrent or consecutive administration in treating or ameliorating one or more symptoms of HCV or disorders associated with HCV in a subject in need thereof.

In one embodiment, the present invention provides medicaments and methods using the same comprising, separately or together: (a) at least one cytochrome P450 isozyme 3A4 (CYP3A4) inhibitor; and (b) at least one anti-HCV agent which is:

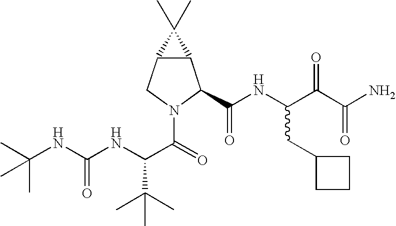

Formula Ia or a pharmaceutically acceptable salt, solvate or ester thereof; with the proviso that when at least one CYP3A4 inhibitor is ritonavir then at least one anti-HCV agent is not Formula Ia; for concurrent or consecutive administration in treating or ameliorating one or more symptoms of HCV or disorders associated with HCV in a subject in need thereof.

In a preferred embodiment, the present invention provides medicaments and methods using the same comprising, separately or together: (a) at least one cytochrome P450 isozyme 3A4 (CYP3A4) inhibitor; and (b) at least one anti-HCV agent which is:

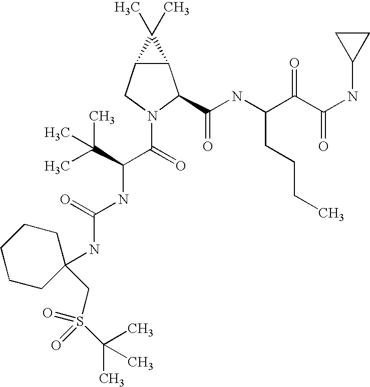

Formula XIVa or a pharmaceutically acceptable salt, solvate or ester thereof; for concurrent or consecutive administration in treating or ameliorating one or more symptoms of HCV or disorders associated with HCV in a subject in need thereof.

In another preferred embodiment, the present invention provides medicaments and methods using the same comprising, separately or together: (a) at least one cytochrome P450 isozyme 3A4 (CYP3A4) inhibitor; and (b) at least one anti-HCV agent which is:

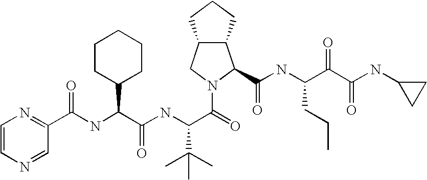

Formula XXVII or a pharmaceutically acceptable salt, solvate or ester thereof; for concurrent or consecutive administration in treating or ameliorating one or more symptoms of HCV or disorders associated with HCV in a subject in need thereof.

In one embodiment, the medicament further comprises at least one other therapeutic agent. In a preferred embodiment, at least one other therapeutic agent is an immunomodulatory agent that enhances an antiviral response such as an interferon or a toll-like receptor (TLR) agonist. In a preferred embodiment, at least one other therapeutic agent is a TLR-7 agonist, such as SM360320 (9-benzyl-8-hydroxy-2-(2-methoxy-ethoxy)adenine). In one embodiment, wherein at least one other therapeutic agent is an interferon, the medicament further comprises ribavirin. In another preferred embodiment, at least one other therapeutic agent is ribavirin. In yet another preferred embodiment, at least one other therapeutic agent is interferon, ribavirin, levovirin, VP 50406, ISIS 14803, HEPTAZYME, VX 497, THYMOSIN, MAXAMINE, mycophenolate mofetil, or an interleukin-10 (IL-10) antagonist or an IL-10 receptor antagonist. In still another preferred embodiment, at least one other therapeutic agent is an antibody specific to IL-10. Preferably, the antibody specific to IL-10 is humanized 12G8.

In one embodiment, at least one CYP3A4 inhibitor is selected from the compounds disclosed in one or more of the following patent applications assigned to Sequoia Pharmaceuticals, Inc., the disclosure of each of which is incorporated herein by reference: U.S. Patent Publication No. US 2005/0209301 and U.S. Patent Publication No. US 2005/0267074.

In one embodiment, at least one CYP3A4 inhibitor is selected from the compounds disclosed in one or more of the following patents and patent applications assigned to Bioavailability Systems, LLC, the disclosure of each of which is incorporated herein by reference: US 2004058982, U.S. Pat. No. 6,248,776, U.S. Pat. No. 6,063,809, U.S. Pat. No. 6,054,477, U.S. Pat. No. 6,162,479, WO 2000054768, U.S. Pat. No. 6,309,687, U.S. Pat. No. 6,476,066, U.S. Pat. No. 6,660,766, WO 2004037827, U.S. Pat. No. 6,124,477, U.S. Pat. No. 5,820,915, U.S. Pat. No. 5,993,887, U.S. Pat. No. 5,990,154, U.S. Pat. No. 6,255,337. In a preferred embodiment, at least one CYP3A4 inhibitor is a compound disclosed in WO 2004037827.

According to certain preferred embodiments of the present invention, at least one CYP3A4 inhibitor is ritonavir, ketoconazole, clarithromycin, BAS 100, a compound disclosed in FIGS. 1A-1G, or a pharmaceutically acceptable salt, solvate or ester thereof. In one embodiment, at least one CYP3A4 inhibitor is ritonavir or a pharmaceutically acceptable salt, solvate or ester thereof. In another embodiment, at least one CYP3A4 inhibitor is ketoconazole or a pharmaceutically acceptable salt, solvate or ester thereof. In another embodiment, at least one CYP3A4 inhibitor is clarithromycin or a pharmaceutically acceptable salt, solvate or ester thereof. In another embodiment, at least one CYP3A4 inhibitor is a compound disclosed in FIGS. 1A-1G or a pharmaceutically acceptable salt, solvate or ester thereof. In another embodiment, at least one CYP3A4 inhibitor is BAS 100 or a pharmaceutically acceptable salt, solvate or ester thereof. In one embodiment, at least one CYP3A4 inhibitor is identified by the Chemical Abstracts Services (CAS) Number 684217-04-7 which corresponds to the Chemical Abstract index name 7H-Furo[3,2-g][1]benzopyran-7-one, 4-[[(2E)-5-[(4R)-4′-[[(2E)-3,7-dimethyl-2,6-octadienyl]oxy]-5,5-dimethylspiro[1,3-dioxolane-2,7′-[7H]furo[3,2-g][1]benzopyran]-4-yl]-3-methyl-2-pentenyl]oxy]; the CAS Number 684217-03-6 which corresponds to the Chemical Abstract index name 7H-Furo[3,2-g][1]benzopyran-7-one, 4-[[(2E)-5-[(4R)-4′-[[2E)-6,7-dihydroxy-3,7-dimethyl-2-octenyl]oxy]-5,5-dimethylspiro[1,3-dioxolane-2,7′-[7H]furo[3,2-g][1]benzopyran]-4-yl]-3-methyl-2-pentenyl]oxy], or the CAS Number 267428-36-4 which corresponds to the Chemical Abstract index name 7H-Furo[3,2-g][1]benzopyran-7-one, 4-[[(2E)-5-[(2R,4R)-4′-[[(2E,6R)-6,7-dihydroxy-3,7-dimethyl-2-octenyl]oxy]-5,5-dimethylspiro[1,3-dioxolane-2,7′-[7H]furo[3,2-g][1]benzopyran]-4-yl]-3-methyl-2-pentenyl]oxy]; all of which is further described in WO 2004037827. In one embodiment, at least one CYP3A4 inhibitor has the structure shown below:

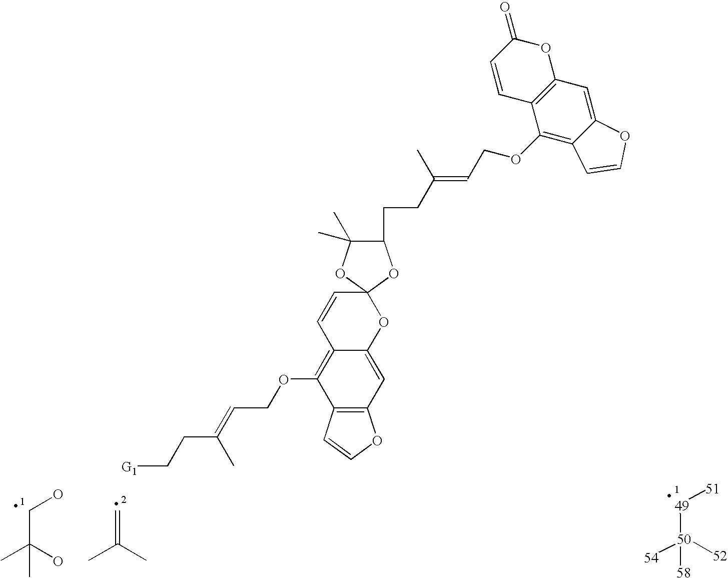

In one embodiment, the HCV protease inhibitor is a compound of Formula I to XXVI detailed below or a pharmaceutically acceptable salt, solvate or ester thereof.

In one embodiment, the HCV protease inhibitor is a compound of structural Formula I:

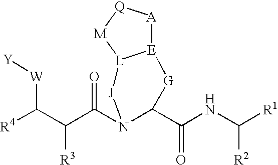

or a pharmaceutically acceptable salt, solvate or ester thereof;
wherein in Formula I:

Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y may be optionally substituted with X¹¹ or X¹²;

X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;

X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is COR⁵, wherein R⁵ is COR⁷ wherein R⁷ is NHR⁹, wherein R⁹ is selected from the group consisting of H, alkyl, aryl, heteroalkyl, heteroaryl, cycloalkyl, cycloalkyl, arylalkyl, heteroarylalkyl, [CH(R^{1′})]ₚCOOR¹¹, [CH(R^{1′})]ₚCONR¹²R¹³, [CH(R^{1′})]ₚSO₂R¹¹, [CH(R^{1′})]ₚCOR¹¹, [CH(R^{1′})]ₚCH(OH)R¹¹, CH(R^{1′})CONHCH(R²)COOR¹¹, CH(R^{1′})CONHCH(R^{2′})CONR¹²R¹³, CH(R^{1′})CONHCH(R²)R′, CH(R^{1′})CONHCH(R^{2′})CONHCH(R^{3′})COOR¹¹CH(R^{1′})CONHCH(R^{2′})CONHCH(R^{3′})CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹ and CH(R^{1′})CONHCH(R^{2′})CONHCH(R^{3′})CONHCH(R^{4′})CONHCH(R^{5′}) CONR¹²R¹³, wherein R^{1′}, R^{2′}, R^{3′}, R^{4′}, R^{5′}, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;

Z is selected from O, N, CH or CR;

W may be present or absent, and if W is present, W is selected from C═O, C═S, C(═N—CN), or SO₂;

Q may be present or absent, and when Q is present, Q is CH, N, P, (CH₂)ₚ, (CHR)ₚ, (CRR′)ₚ, O, NR, S, or SO₂; and when Q is absent, M may be present or absent; when Q and M are absent, A is directly linked to L;

A is O, CH₂, (CHR)ₚ, (CHR—CHR′)ₚ, (CRR′)ₚ, NR, S, SO₂ or a bond;

E is CH, N, CR, or a double bond towards A, L or G;

G may be present or absent, and when G is present, G is (CH₂)ₚ, (CHR)ₚ, or (CRR′)ₚ; and when G is absent, J is present and E is directly connected to the carbon atom in Formula I as G is linked to;

J may be present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;

L may be present or absent, and when L is present, L is CH, CR, O, S or NR; and when L is absent, then M may be present or absent; and if M is present with L being absent, then M is directly and independently linked to E, and J is directly and independently linked to E;

M may be present or absent, and when M is present, M is O, NR, S, SO₂, (CH₂)ₚ, (CHR)ₚ (CHR—CHR′)ₚ, or (CRR′)ₚ;

p is a number from 0 to 6; and

R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen; (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;

further wherein said unit N—C-G-E-L-J-N represents a five-membered or six-membered cyclic ring structure with the proviso that when said unit N—C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring.

In another embodiment, the HCV protease inhibitor is a compound of structural Formula II:

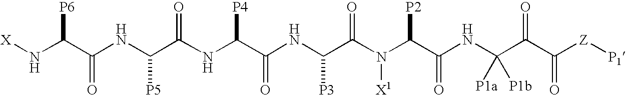

or a pharmaceutically acceptable salt, solvate or ester thereof;
wherein in Formula II:

Z is NH;

X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylalkylcarbonyl, arylcarbonyl, heteroarylcarbonyl, alkoxycarbonyl, heterocyclyloxycarbonyl, aryloxycarbonyl, heteroaryloxycarbonyl, alkylaminocarbonyl, heterocyclylaminocarbonyl, arylaminocarbonyl, or heteroarylaminocarbonyl moiety: with the proviso that X may be additionally optionally substituted with R¹² or R¹³;

X¹ is H; C₁-C₄ straight chain alkyl; C₁-C₄ branched alkyl or CH₂-aryl (substituted or unsubstituted);

R¹² is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R¹² may be additionally optionally substituted with R¹³.

R¹³ is hydroxy, alkoxy, aryloxy, thio, alkylthio; arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³.

P1a, P1b, P2, P3, P4, P5, and P6 are independently: H; C₁-C₁₀ straight or branched chain alkyl; C₂-C₁₀ straight or branched chain alkenyl; C₃-C₈ cycloalkyl, C₃-C₈ heterocyclic; (cycloalkyl)alkyl or (heterocyclyl)alkyl, wherein said cycloalkyl is made up of 3 to 8 carbon atoms, and zero to 6 oxygen, nitrogen, sulfur, or phosphorus atoms, and said alkyl is of 1 to 6 carbon atoms; aryl, heteroaryl, arylalkyl, or heteroarylalkyl, wherein said alkyl is of 1 to 6 carbon atoms;

wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and

P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R¹³.

In another embodiment, the HCV protease inhibitor is a compound of structural Formula III:

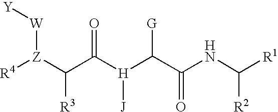

or a pharmaceutically acceptable salt, solvate or ester thereof;
wherein in Formula III:

G is carbonyl;

J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y may be additionally optionally substituted with X¹¹ or X¹²;

X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;

X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is COR⁵ or C(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl, CH(R¹′)COOR¹¹, CH(R¹′)CONR¹²R¹³, CH(R¹′)CONHCH(R²′)COOR¹¹, CH(R¹′)CONHCH(R²′)CONR¹²R¹³, CH(R¹′)CONHCH(R²′)R′, CH(R¹′)CONHCH(R²′)CONHCH(R³′)COOR¹¹, CH(R¹′)CONHCH(R²′)CONHCH(R³′)CONR¹²R¹³, CH(R¹′)CONHCH(R²′)CONHCH(R³′)CONHCH(R⁴′)COOR¹¹, CH(R¹′)CONHCH(R²′)CONHCH(R³′)CONHCH(R⁴′)CONR¹²R¹³, CH(R^{1′})CONHCH(R²′)CONHCH(R³′)CONHCH(R⁴′)CONHCH(R⁵′)COOR¹¹, and CH(R¹′)CONHCH(R^{2′})CONHCH(R³′)CONHCH(R⁴′)CONHCH(R^{5′}) CONR¹²R¹³, wherein R¹′, R^{2′}, R³′, R⁴′, R⁵′, R¹¹, R¹², R¹³, and R′ may be the same or different and are independently selected from a group consisting of H, alkyl, aryl, heteroalkyl, heteroaryl, cycloalkyl, alkyl-aryl, alkyl-heteroaryl, aryl-alkyl and heteroaralkyl;

Z is selected from O, N, or CH;

W may be present or absent, and if W is present, W is selected from C═O, C═S, or SO₂; and

R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate.

In another embodiment, the HCV protease inhibitor is a compound of structural Formula IV:

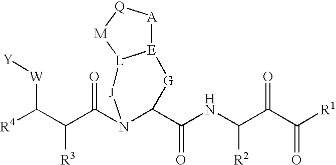

or a pharmaceutically acceptable salt, solvate or ester thereof;
wherein in Formula IV:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y may be optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is selected from the following structures:

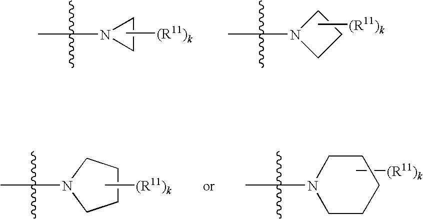

wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹≠H) may be optionally substituted with X¹¹ or X¹²;

Z is selected from O, N, CH or CR;
W may be present or absent, and if W is present, W is selected from C═O, C═S, C(═N—CN), or S(O₂);
Q may be present or absent, and when Q is present, Q is CH, N, P, (CH₂)ₚ, (CHR)ₚ, (CRR′)ₚ, O, N(R), S, or S(O₂); and when Q is absent, M may be present or absent;
when Q and M are absent, A is directly linked to L;
A is O, CH₂, (CHR)ₚ, (CHR—CHR)ₚ, (CRR)ₚ, N(R), S, S(O₂) or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J may be present or absent, and when J is present, J is (CH₂)ₚ, (CHR)ₚ, or (CRR′)ₚ, S(O₂), NH, N(R) or O; and when J is absent, G is present and E is directly linked to N shown in Formula I as linked to J;
L may be present or absent, and when L is present, L is CH, C(R), O, S or N(R); and when L is absent, then M may be present or absent; and if M is present with L being absent, then M is directly and independently linked to E, and J is directly and independently linked to E;
M may be present or absent, and when M is present, M is O, N(R), S, S(O₂), (CH₂)ₚ, (CHR)ₚ(CHR—CHR′)ₚ, or (CRR′)ₚ;
p is a number from 0 to 6; and
R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered cyclic ring structure or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring.

In another embodiment, the HCV protease inhibitor is a compound of structural Formula V:

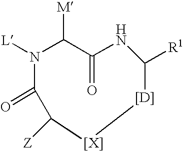

or a pharmaceutically acceptable salt, solvate or ester thereof;
wherein in Formula V:

### (1) R¹ is —C(O)R⁵ or —B(OR)₂;

(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;

### (3) R⁷ is H, —OH, —OR⁸, or —CHR⁹R¹⁰;

(4) R⁸, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R^{1′})CH(R^{1′})C(O)OR¹¹, [CH(R^{1′})]ₚC(O)OR¹¹, —[CH(R^{1′})]ₚC(O)NR¹²R¹³, —[CH(R^{1′)]}ₚS(O₂)R¹¹, —[CH(R^{1′})]ₚC(O)R¹¹, —[CH(R^{1′})]ₚS(O₂)NR¹²R¹³, CH(R^{1′})C(O)N(H)CH(R^{2′})(R′), CH(R^{1′})CH(R^{1′})C(O)NR¹²R¹³, —CH(R^{1′})CH(R^{1′})S(O₂)R¹¹, —CH(R^{1′})CH(R^{1′})S(O₂)NR¹²R¹³, —CH(R^{1′})CH(R^{1′})C(O)R¹¹, —[CH(R^{1′})]ₚCH(OH)R¹¹, —CH(R^{1′})C(O)N(H)CH(R^{2′})C(O)OR¹¹, C(O)N(H)CH(R^{2′})C(O)OR¹¹, —C(O)N(H)CH(R^{2′})C(O)R¹¹, CH(R^{I})C(O)N(H)CH(R^{2′}) C(O)NR¹²R¹³, —CH(R^{1′})C(O)N(H)CH(R^{2′})R′, CH(R^{1′})C(O)N(H)CH(R^{2′})C(O)N(H)CH(R^{3′})C(O)OR¹¹, CH(R^{1′})C(O)N(H)CH(R^{2′})C(O)CH(R^{3′})NR¹²R¹³, CH(R^{1′})C(O)N(H)CH(R^{2′})C(O)N(H)CH(R^{3′})C(O)NR¹²R¹³, CH(R^{1′})C(O)N(H)CH(R^{2′})C(O)N(H)CH(R^{3′})C(O)N(H)CH(R^{4′})C(O)OR¹¹, H(R^{1′})C(O)N(H)CH(R^{2′})C(O)N(H)CH(R^{3′})C(O)N(H)CH(R^{4′})C(O)NR¹²R¹³, CH(R^{1′})C(O)N(H)CH(R^{2′})C(O)N(H)CH(R^{3′})C(O)N(H)CH(R⁴)C(O)N(H)CH(R^{5′})C(O)OR¹¹, and CH(R^{1′})C(O)N(H)CH(R^{2′})C(O)N(H)CH(R^{3′})C(O)N(H)CH(R^{4′})C(O)N(H)CH(R^{5′}) C(O)NR¹²R¹³;
wherein R^{1′}, R^{2′}, R^{3′}, R^{4′}, R^{5′}, R¹¹, R¹² and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl; or
R¹² and R¹³ are linked together wherein the combination is cycloalkyl, heterocycloalkyl, aryl or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain;
or L′ and M′ are linked together to form a ring structure wherein the portion of structural Formula 1 represented by:

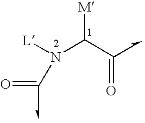

and wherein structural Formula 2 is represented by:

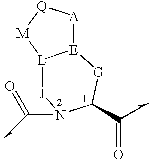

wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;
L is present or absent, and when L is present, L is C(H) or C(R); when L is absent, M is present or absent; if M is present with L being absent, then M is directly and independently linked to E, and J is directly and independently linked to E;
G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;
Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent being selected from —OR, —CH(R)(R′), S(O)₀₋₂R or —NRR′ or (iii) absent; when both Q and M are absent, A is either directly linked to L, or A is an independent substituent on E, said independent substituent being selected from —OR, —CH(R)(R′), S(O)₀₋₂R or —NRR′ or A is absent;
A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, O, OR, N(R), S, S(O₂), (CH₂)ₚ, (HR)ₚ (CHR—CHR′)ₚ, or (CRR′)ₚ; or M is linked to A to form an alicyclic, aliphatic or heteroalicyclic bridge;
(8) Z′ is represented by the structural Formula 3:

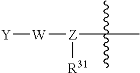

wherein in Formula 3:
Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹¹ is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;

### Z is O, N, C(H) or C(R);

R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and
R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
W may be present or absent, and if W is present, W is C(═O), C(═S), C(═N—CN), or S(O₂);
(9) X is represented by structural Formula 4:

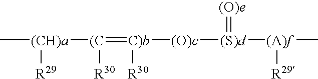

wherein in Formula 4:
a is 2, 3, 4, 5, 6, 7, 8 or 9;
b, c, d, e and f are 0, 1, 2, 3, 4 or 5;

### A is C, N, S or O;

R²⁹ and R^{29′} are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R²⁹ and R^{29′} are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;
(10) D is represented by structural Formula 5:

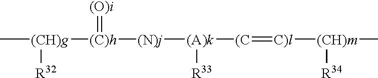

wherein in Formula 5:
R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R³² and R³⁴ are linked together such that the combination forms a portion of a cycloalkyl group;
g is 1, 2, 3, 4, 5, 6, 7, 8 or 9;
h, i, j, k, l and m are 0, 1, 2, 3, 4 or 5; and

### A is C, N, S or O,

(11) provided that when structural Formula 2:

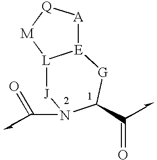

### Formula 2

is

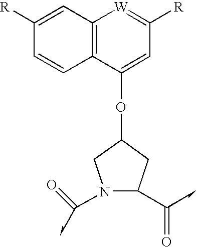

and

W′ is CH or N, both the following conditional exclusions (i) and (ii) apply: conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆ alkyl or C₃₋₆ cycloalkyl;

and

conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₆ alkyl, C₃₋₆ cycloalkyl, C_{6 to 10} aryl or C₇₋₁₆ aralkyl.

In another embodiment, the HCV protease inhibitor is a compound of structural Formula VI:

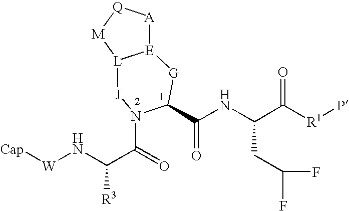

or a pharmaceutically acceptable salt, solvate or ester thereof;
wherein in Formula VI:

Cap is H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arylalkyloxy or heterocyclylamino can be unsubstituted or optionally independently substituted with one or two substituents which can be the same or different and are independently selected from X¹ and X²

P′ is —NHR;

X′ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;

X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;

W may be present or absent, and when W is present W is C(═O), C(═S), C(═NH), C(═N—OH), C(═N—CN), S(O) or S(O₂);

Q may be present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R′), S(O)₀₋₂R or —NRR′; when both Q and M are absent, A is either directly linked to L, or A is an independent substituent on E, selected from —OR, CH(R)(R′), —S(O)₀₋₂R or —NRR′;

A is present or absent and if present A is —O—, —O(R)CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either Q and E are connected by a bond or Q is an independent substituent on M;

E is present or absent and if present E is CH, N, C(R);

G may be present or absent, and when G is present, G is (CH₂)ₚ, (CHR)ₚ, or (CRR′)ₚ; when G is absent, J is present and E is directly connected to the carbon atom marked position 1;

J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;

L may be present or absent, and when L is present, L is CH, N, or CR; when L is absent, M is present or absent; if M is present with L being absent, then M is directly and independently linked to E, and J is directly and independently linked to E;

M may be present or absent, and when M is present, M is O, N(R), S, S(O₂), (CH₂)ₚ, (CHR)ₚ, (CHR—CHR′)ₚ, or (CRR′)ₚ;

p is a number from 0 to 6;

R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;

R and R′ in (CRR′) can be linked together such that the combination forms a cycloalkyl or heterocyclyl moiety; and

R¹ is carbonyl.

In another embodiment, the HCV protease inhibitor is a compound of structural Formula VII:

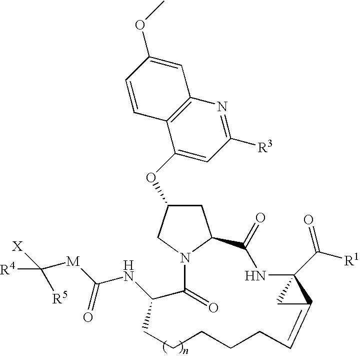

or a pharmaceutically acceptable salt, solvate or ester thereof;
wherein in Formula VII:

M is O, N(H), or CH₂;

n is 0-4;

R¹ is —OR⁶, —NR⁶R⁷ or

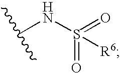

where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;

R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety

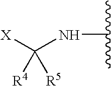

is represented by

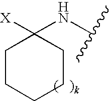

where k is 0 to 2;
X is selected from the group consisting of:

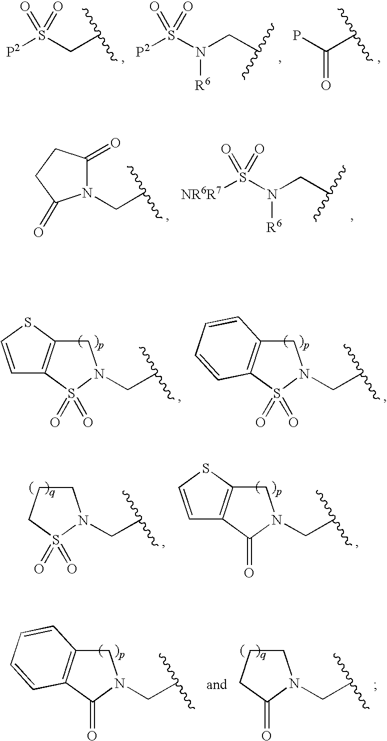

where p is 1 to 2, q is 1-3 and P² is alkyl, aryl, heteroaryl, heteroalkyl, cycloalkyl, dialkylamino, alkylamino, arylamino or cycloalkylamino;

and
R³ is selected from the group consisting of: aryl, heterocyclyl, heteroaryl,

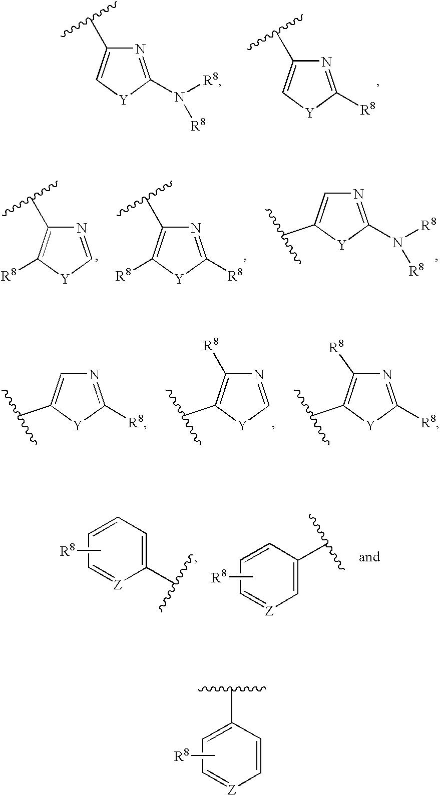

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the HCV protease inhibitor is a compound of structural Formula VIII:

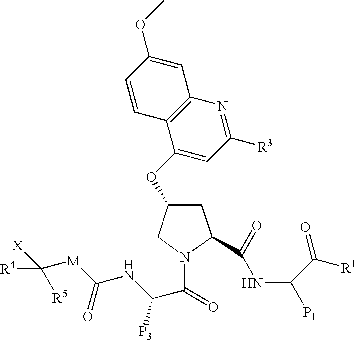

or a pharmaceutically acceptable salt, solvate or ester thereof;
wherein in Formula VIII:

M is O, N(H), or CH₂;

R¹ is —C(O)NHR⁶, where R⁶ is hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino or alkylamino;

P₁ is selected from the group consisting of alkyl, alkenyl, alkynyl, cycloalkyl haloalkyl;

P₃ is selected from the group consisting of alkyl, cycloalkyl, aryl and cycloalkyl fused with aryl;

R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety

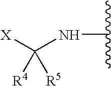

is represented by

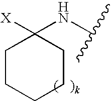

where k is 0 to 2;

X is selected from the group consisting of:

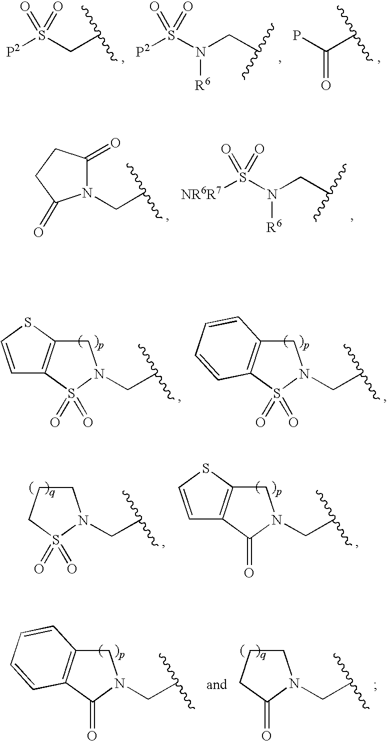

where p is 1 to 2, q is 1 to 3 and P² is alkyl, aryl, heteroaryl, heteroalkyl, cycloalkyl, dialkylamino, alkylamino, arylamino or cycloalkylamino; and

R³ is selected from the group consisting of: aryl, heterocyclyl, heteroaryl,

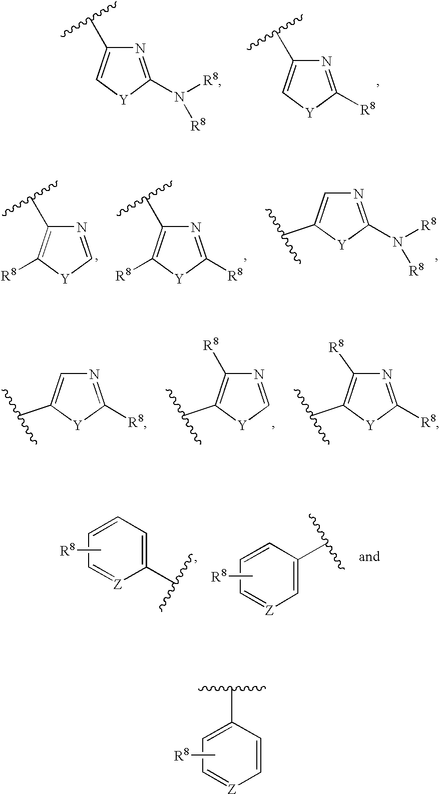

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the HCV protease inhibitor is a compound of structural Formula IX:

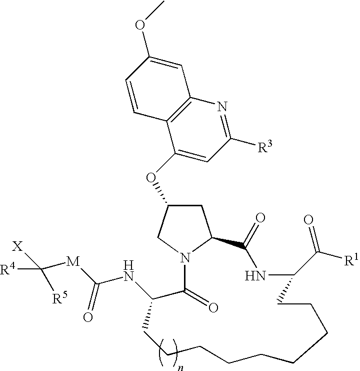

or a pharmaceutically acceptable salt, solvate or ester thereof;
wherein in Formula IX:

M is O, N(H), or CH₂;

n is 0-4;

R¹ is —OR⁶, —NR⁶R⁷ or

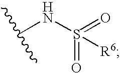

where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;

R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety

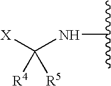

is represented by

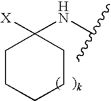

where k is 0 to 2;
X is selected from the group consisting of:

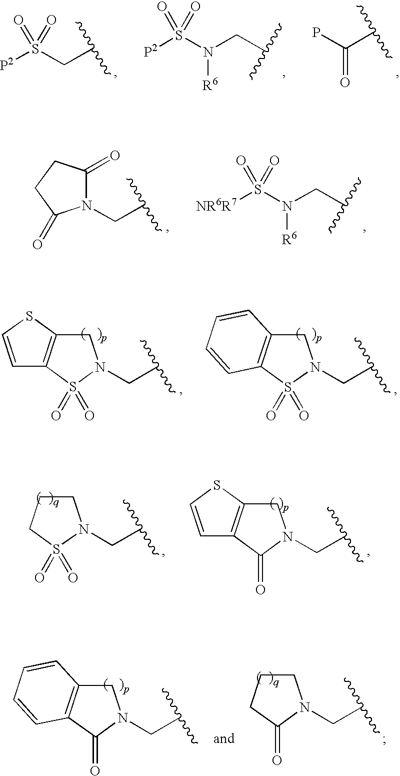

where p is 1 to 2, q is 1 to 3 and P² is alkyl, aryl, heteroaryl, heteroalkyl, cycloalkyl, dialkylamino, alkylamino, arylamino or cycloalkylamino;

and
R³ is selected from the group consisting of: aryl, heterocyclyl, heteroaryl,

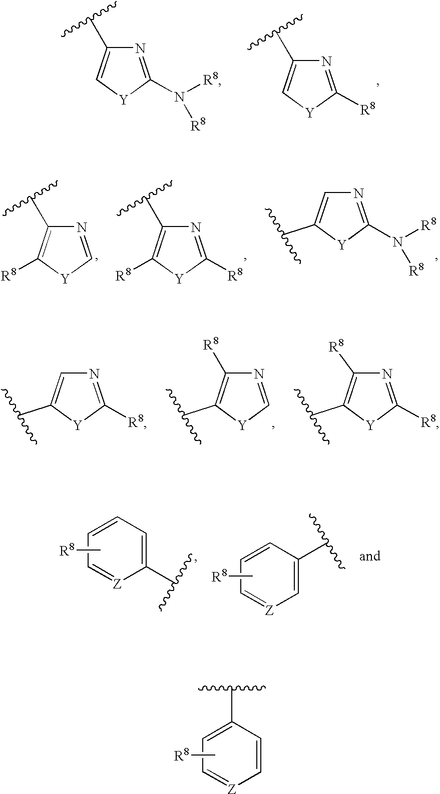

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the HCV protease inhibitor is a compound of structural Formula X:

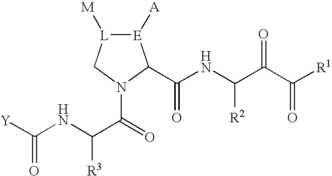

or a pharmaceutically acceptable salt, solvate or ester thereof;
wherein in Formula X:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other such that the moiety:

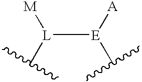

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties:

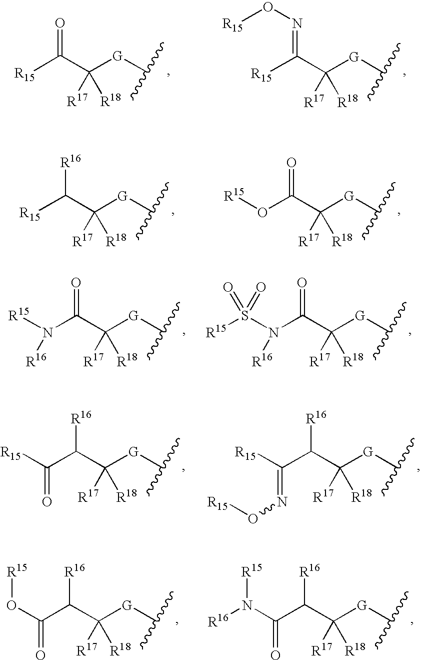

wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In one embodiment, the HCV protease inhibitor is a compound of structural Formula XI:

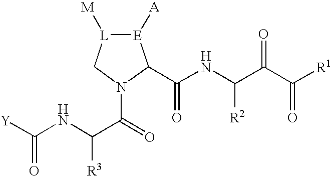

or a pharmaceutically acceptable salt, solvate or ester thereof;
wherein in Formula XI:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, NR⁹R¹⁰, SR, SO₂R, and halo; or A and M are connected to each other (in other words, A-E-L-M taken together) such that the moiety:

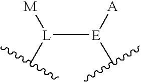

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;

Y is selected from the following moieties:

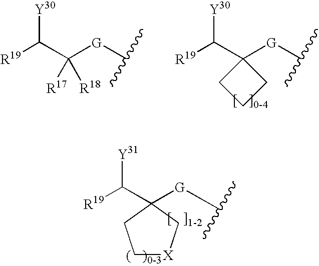

wherein Y³⁰ and Y³¹ are selected from

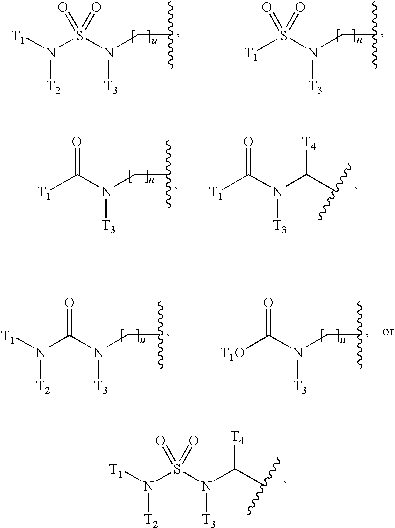

where u is a number 0-6;

X is selected from O, NR¹⁵, NC(O)R¹⁶, S, S(O) and SO₂;

G is NH or O; and

R¹⁵, R¹⁶, R¹⁷, R¹⁸, R¹⁹, T₁, T₂, T₃ and T₄ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XII:

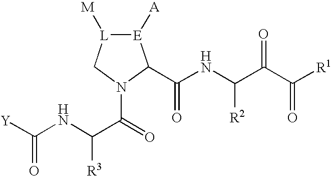

or a pharmaceutically acceptable salt, solvate or ester thereof;
wherein in Formula XII:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other such that the moiety:

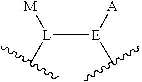

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties:

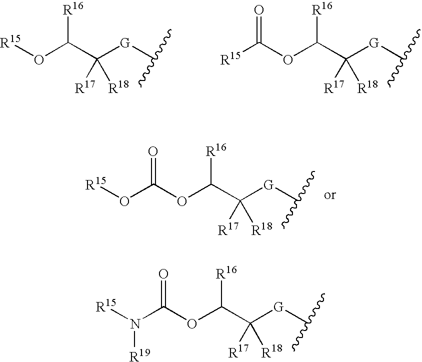

wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XIII:

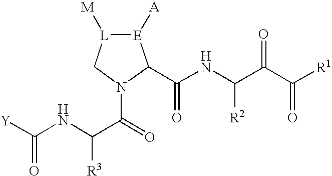

or a pharmaceutically acceptable salt, solvate or ester thereof;
wherein in Formula XIII:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other (in other words, A-E-L-M taken together) such that the moiety:

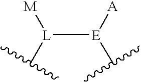

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties:

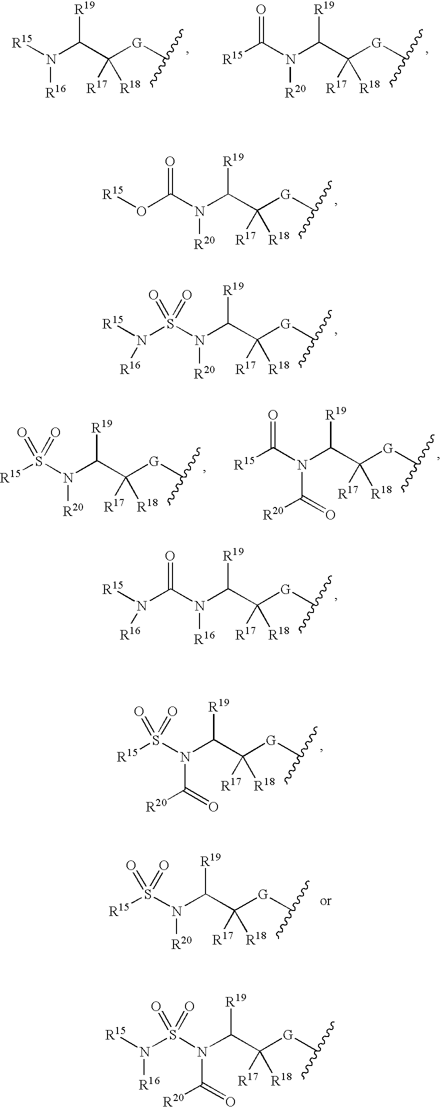

wherein G is NH or O, and R¹⁵, R¹⁶, R¹⁷, R¹⁸, R¹⁹ and R²⁰ can be the same or different, each being independently selected from the group consisting of H, C₁-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XIV:

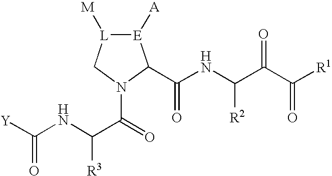

or a pharmaceutically acceptable salt, solvate or ester thereof;
wherein in Formula XIV:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo;

or A and M are connected to each other such that the moiety:

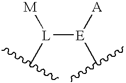

shown above in Formula I forms either a three, four, six, seven or eight-membered cycloalkyl, a four to eight-membered heterocyclyl, a six to ten-membered aryl, or a five to ten-membered heteroaryl;

E is C(H) or C═;

L is C(H), C═, CH₂C═, or C═CH₂;

R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;

and Y is selected from the following moieties:

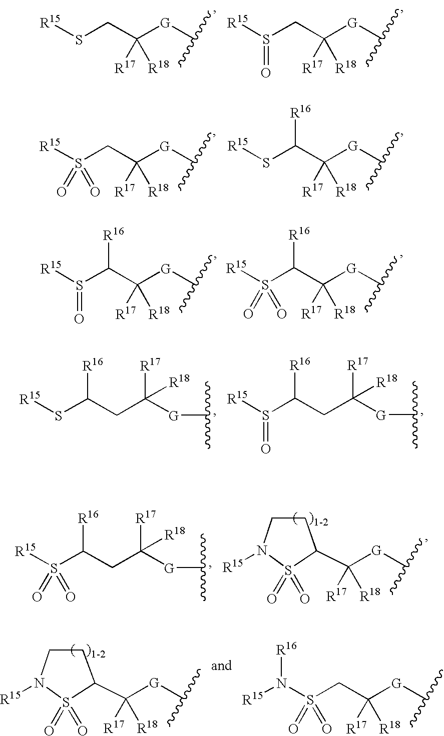

wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XV:

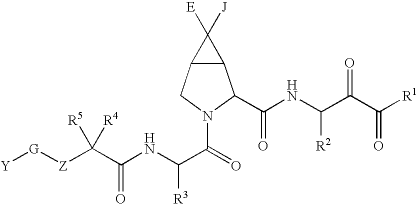

or a pharmaceutically acceptable salt, solvate or ester thereof;
wherein in Formula XV:

R¹ is NHR⁹, wherein R⁹ is H, alkyl-, aryl-, heteroalkyl-, heteroaryl-, cycloalkyl-, cycloalkyl-, arylalkyl-, or heteroarylalkyl;

E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;

Z is N(H), N(R), or O, with the proviso that when Z is O, G is present or absent and if G is present with Z being 0, then G is C(═O);

G may be present or absent, and if G is present, G is C(═O) or S(O₂), and when G is absent, Z is directly connected to Y;

Y is selected from the group consisting of:

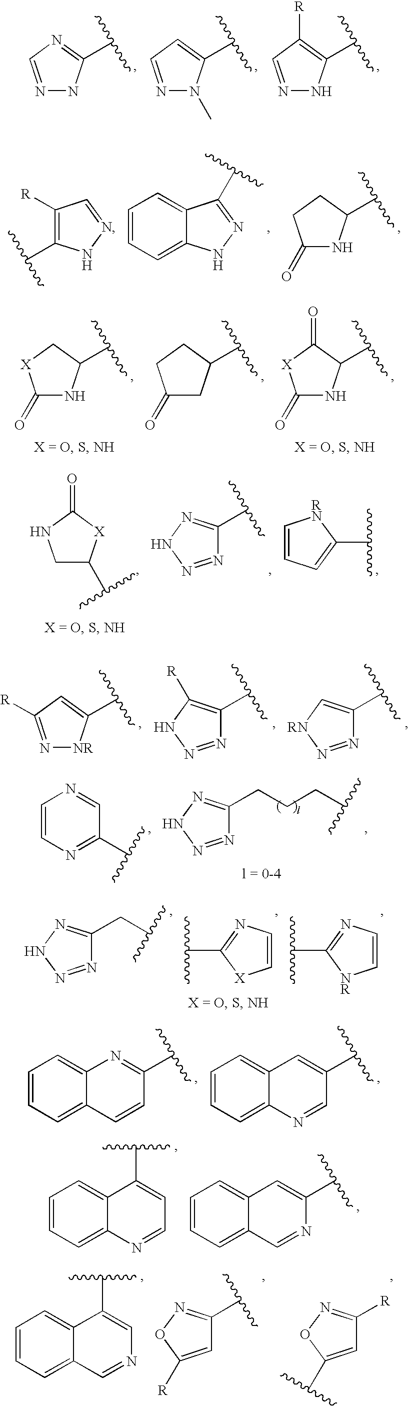

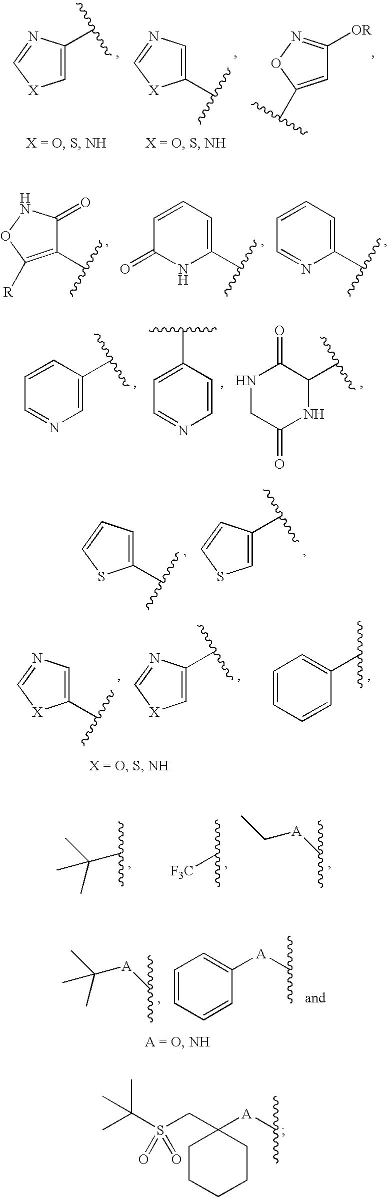

R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;

wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate.

In another embodiment, the HCV protease inhibitor is a compound of structural Formula XVI:

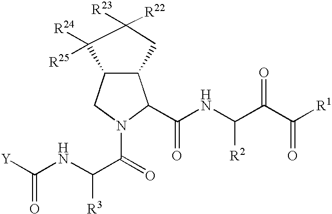

or a pharmaceutically acceptable salt, solvate or ester thereof;
wherein in Formula XVI:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties:

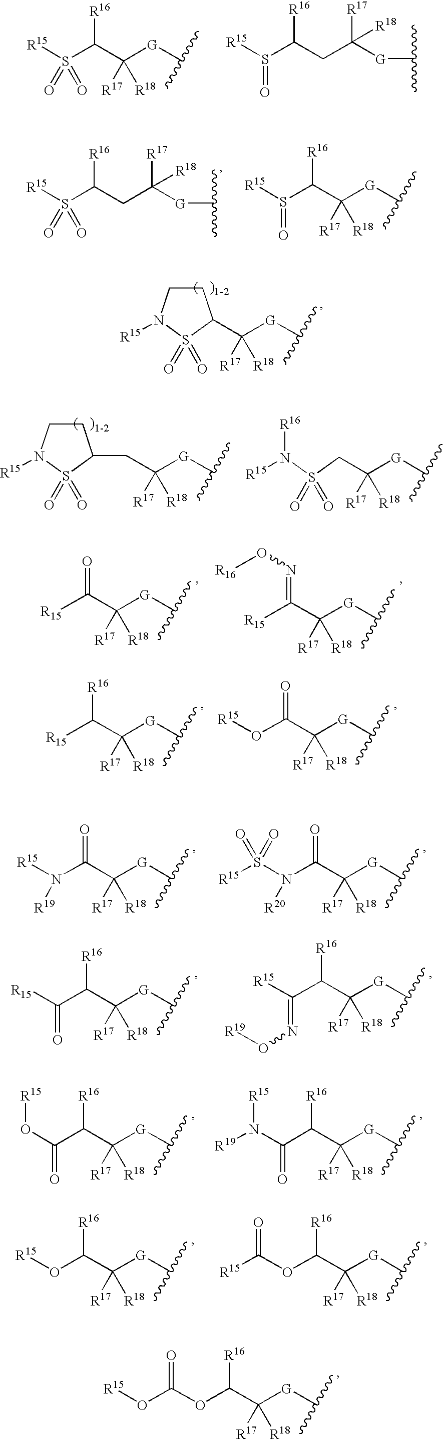

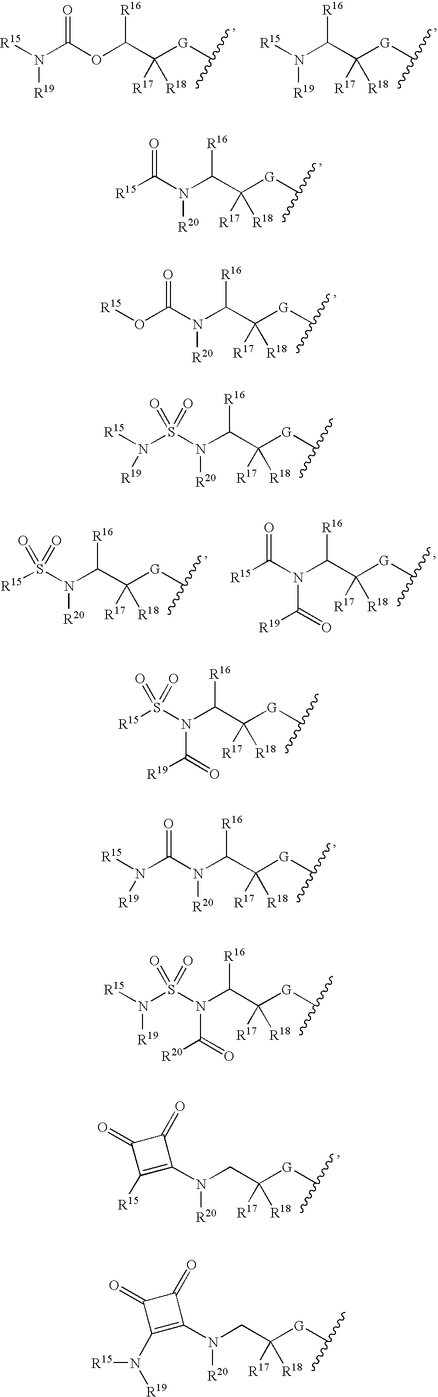

wherein G is NH or O; and R¹⁵, R¹⁶, R¹⁷, R¹⁸, R¹⁹, R²⁰, R²¹, R²², R²³, R²⁴ and R²⁵ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl;

wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XVII:

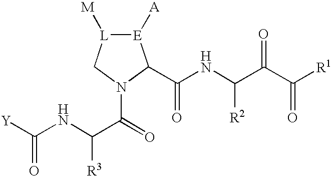

or a pharmaceutically acceptable salt, solvate or ester thereof;
wherein in Formula XVII:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other such that the moiety:

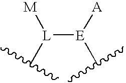

shown above in Formula I forms either a three, four, six, seven or eight-membered cycloalkyl, a four to eight-membered heterocyclyl, a six to ten-membered aryl, or a five to ten-membered heteroaryl;

E is C(H) or C═;

L is C(H), C═, CH₂C═, or C═CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

Y is selected from the following moieties:

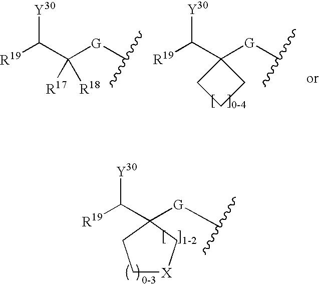

wherein Y³⁰ is selected from

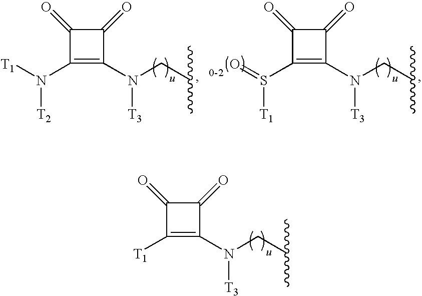

where u is a number 0-1;

X is selected from O, NR¹⁵, NC(O)R¹⁶, S, S(O) and SO₂;

G is NH or O; and

R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XVIII:

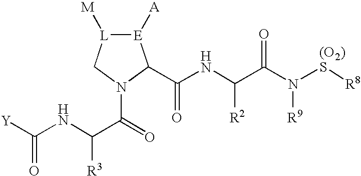

or a pharmaceutically acceptable salt, solvate or ester thereof;
wherein in Formula XVIII:
R⁸ is selected from the group consisting of alkyl-, aryl-, heteroalkyl-, heteroaryl-, cycloalkyl-, heterocyclyl-, arylalkyl-, heteroarylalkyl-, and heterocyclylalkyl;
R⁹ is selected from the group consisting of H, alkyl, alkenyl, alkynyl, aryl and cycloalkyl; A and M can be the same or different, each being independently selected from R, OR, N(H)R, N(RR′), SR, S(O₂)R, and halo; or A and M are connected to each other (in other words, A-E-L-M taken together) such that the moiety:

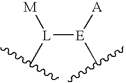

shown above in Formula I forms either a three, four, five, six, seven or eight-membered cycloalkyl, a four to eight-membered heterocyclyl, a six to ten-membered aryl, or a five to ten-membered heteroaryl;

### E is C(H) or C(R);

### L is C(H), C(R), CH₂C(R), or C(R)CH₂;

R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl;
R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;

Y is selected from the following moieties:

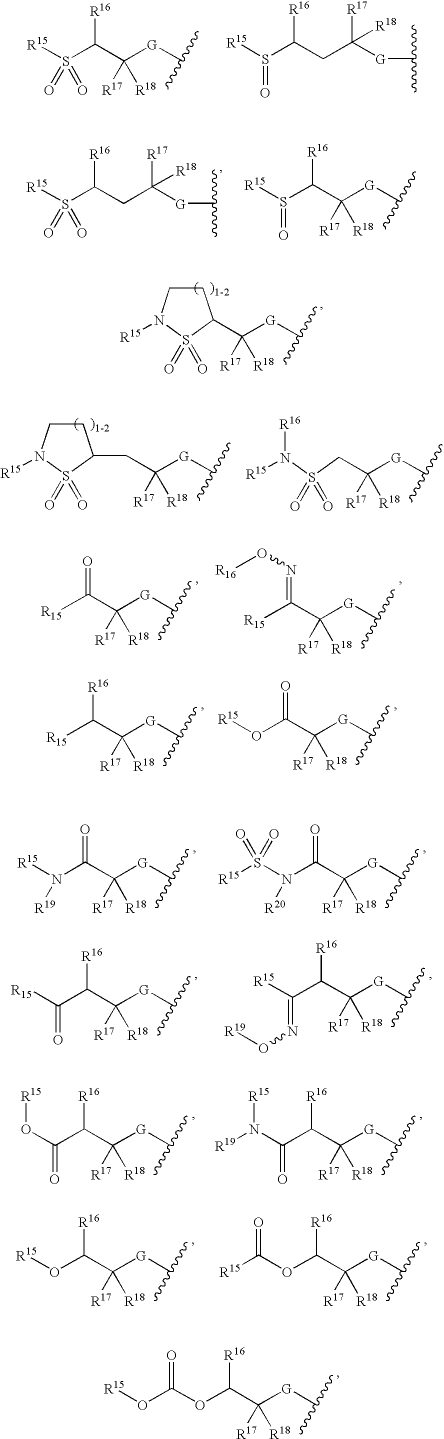

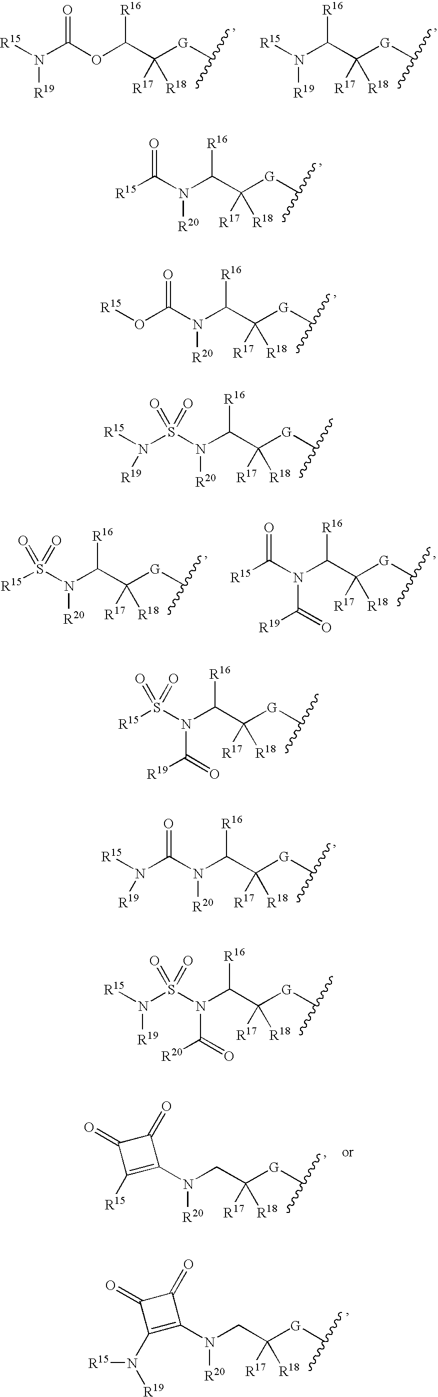

wherein G is NH or O; and R¹⁵, R¹⁶, R¹⁷, R¹⁸, R¹⁹ and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;

wherein each of said alkyl, aryl, heteroaryl, cycloalkyl, spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XIX:

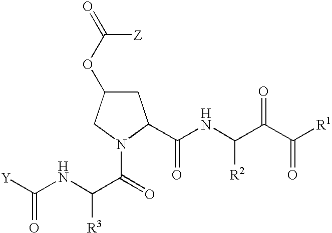

or a pharmaceutically acceptable salt, solvate or ester thereof;
wherein in Formula XIX:

Z is selected from the group consisting of a heterocyclyl moiety, N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N(heteroaryl)₂;

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties:

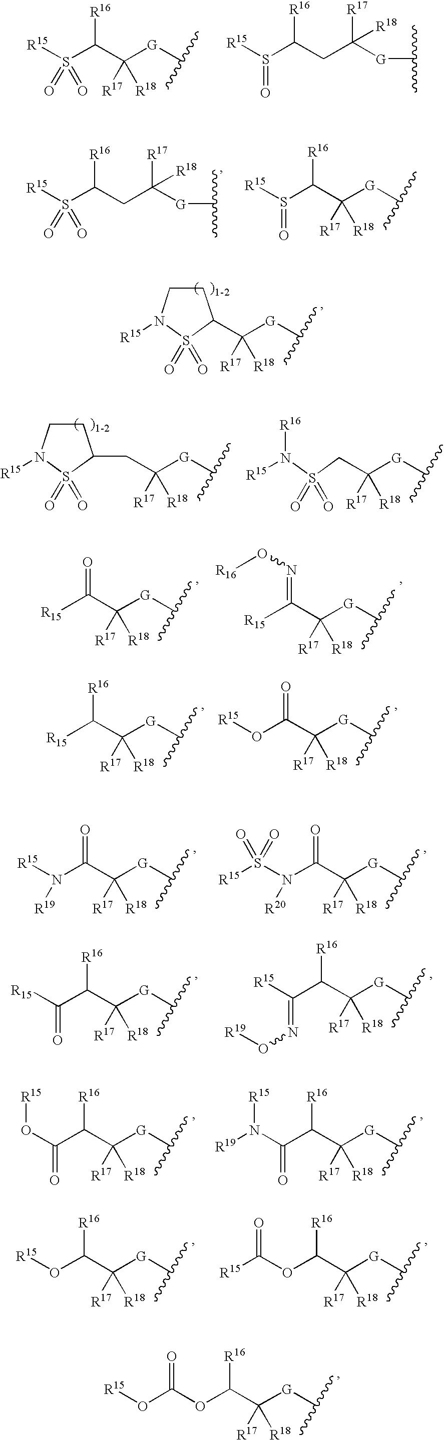

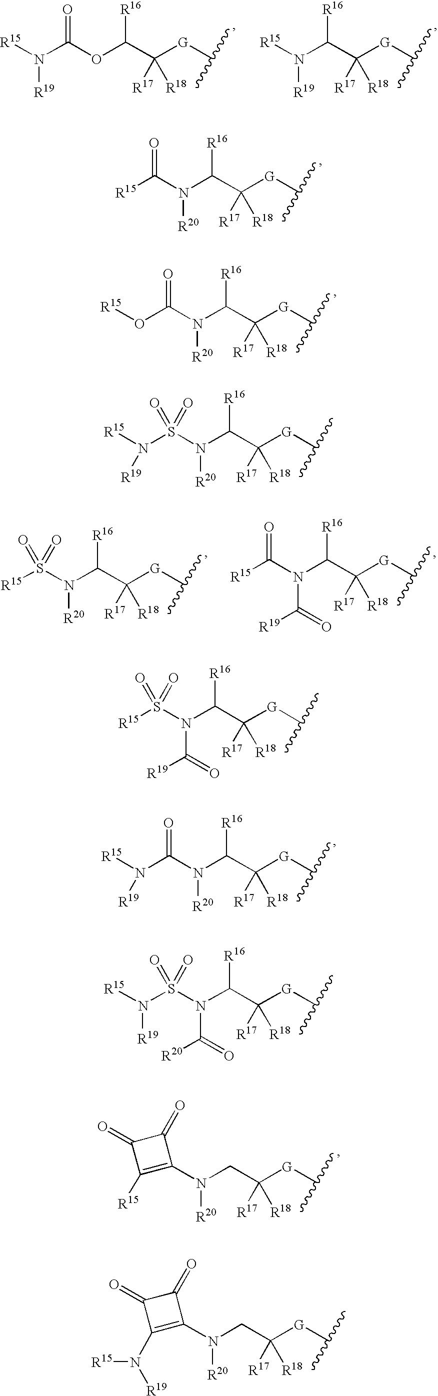

wherein G is NH or O; and R¹⁵, R¹⁶, R¹⁷, R¹⁸, R¹⁹, R²⁰ and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;

wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of structural Formula XX:

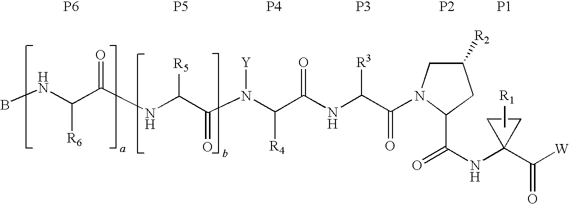

or a pharmaceutically acceptable salt, solvate or ester thereof;
wherein in Formula XX:
a is 0 or 1; b is 0 or 1; Y is H or C₁₋₆ alkyl;
B is H, an acyl derivative of formula R₇—C(O)— or a sulfonyl of formula R₇—SO₂ wherein
R₇ is (i) C₁₋₁₀ alkyl optionally substituted with carboxyl, C₁₋₆ alkanoyloxy or C₁₋₆ alkoxy;

(ii) C₃₋₇ cycloalkyl optionally substituted with carboxyl, (C₁₋₆ alkoxy)carbonyl or phenylmethoxycarbonyl;

(iii) C₆ or C₁₀ aryl or C₇₋₁₆ aralkyl optionally substituted with C₁₋₆ alkyl, hydroxy, or amino optionally substituted with C₁₋₆ alkyl; or

(iv) Het optionally substituted with C₁₋₆ alkyl, hydroxy, amino optionally substituted with C₁₋₆ alkyl, or amido optionally substituted with C₁₋₆ alkyl;

R₆, when present, is C₁₋₆ alkyl substituted with carboxyl;
R₅, when present, is C₁₋₆ alkyl optionally substituted with carboxyl;
R₄ is C₁₋₁₀ alkyl, C₃₋₇ cycloalkyl or C₄₋₁₀ (alkylcycloalkyl);
R₃ is C₁₋₁₀ alkyl, C₃₋₇ cycloalkyl or C₄₋₁₀ (alkylcycloalkyl);
R₂ is CH₂—R₂₀, NH—R₂₀, 0-R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri-substituted with R₂₁, or R₂₀ is Het or (lower alkyl)-Het optionally mono-, di- or tri-substituted with R₂₁, wherein each R₂₁ is independently C₁₋₆ alkyl; C₁₋₆alkoxy; amino optionally mono- or di-substituted with C₁₋₆ alkyl; sulfonyl; NO₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;
wherein R₂₂ is C₁₋₆alkyl; C₁₋₆ alkoxy; amino optionally mono- or di-substituted with C₁₋₆ alkyl; sulfonyl; NO₂; OH; SH; halo; haloalkyl; carboxyl; amide or (lower alkyl)amide;
R₁ is C₁₋₆ alkyl or C₂₋₆ alkenyl optionally substituted with halogen; and
W is hydroxy or a N-substituted amino.

In the above-shown structure of the compound of Formula XX, the terms P6, P5, P4, P3, P2 and P1 denote the respective amino acid moieties as is conventionally known to those skilled in the art.

In another embodiment, the HCV protease inhibitor is a compound of structural Formula XXI:

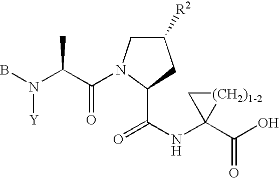

or a pharmaceutically acceptable salt, solvate or ester thereof;
wherein in Formula XXI:
B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl of formula R₄—O—C(O)—; an amide of formula R₄—N(R₅)—C(O)—; a thioamide of formula R₄—N(R₅)—C(S)—; or a sulfonyl of formula R₄—SO₂ wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with 6 alkyl;

R₅ is H or C₁₋₆ alkyl;
with the proviso that when R₄ is an amide or a thioamide, R₄ is not (ii) a cycloalkoxy;
Y is H or C₁₋₆ alkyl;
R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,
or R₂₀ is Het or (lower alkyl)-Het, both optionally mono-, di- or tri-substituted with R₂₁,

wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;

R₁ is H; C₁₋₆ alkyl, C₃₋₇ cycloalkyl, C₂₋₆ alkenyl, or C₂₋₆ alkynyl, all optionally substituted with halogen.

In another embodiment, the HCV protease inhibitor is a compound of structural Formula XXII:

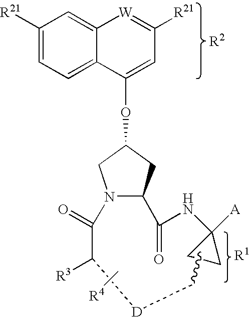

or a pharmaceutically acceptable salt, solvate or ester thereof;
wherein in Formula XXII:

### W is CH or N,

R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂, wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R^{24′} wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵′ wherein each R²⁵ is independently: H, C₁₋₆ alkyl or C₃₋₆ cycloalkyl;
or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹′ wherein R⁴¹ is H, C₁₋₆ alkyl, C₃₋₆ cycloalkyl or —C(O)—R⁴², wherein R⁴² is C₁₋₆ alkyl, C₃₋₆ cycloalkyl or C_{6 or 10} aryl;
R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C₁₋₆ thioalkyl, and
A is an amide of formula —C(O)—NH—R⁵, wherein R⁵ is selected from the group consisting of: C₁₋₆ alkyl, C₃₋₆ cycloalkyl, C_{6 or 10} aryl and C₇₋₁₆ aralkyl;
or A is a carboxylic acid.

In another embodiment, the HCV protease inhibitor is a compound of structural Formula XXIII:

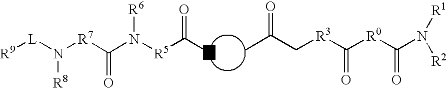

or a pharmaceutically acceptable salt, solvate or ester thereof;
wherein in Formula XXIII:
R⁰ is a bond or difluoromethylene;
R¹ is hydrogen;
R² and R⁹ are each independently optionally substituted aliphatic group, optionally substituted cyclic group or optionally substituted aromatic group;
R³, R⁵ and R⁷ are each independently:

optionally substituted (1,1- or 1,2-)cycloalkylene; or

optionally substituted (1,1- or 1,2-) heterocyclylene; or

methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or; R⁴, R⁶, R⁸ and R¹⁰ are each independently hydrogen or optionally substituted aliphatic group;

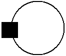

is substituted monocyclic azaheterocyclyl or optionally substituted multicyclic azaheterocyclyl, or optionally substituted multicyclic azaheterocyclenyl wherein the unsaturation is in the ring distal to the ring bearing the R⁹-L-(N(R⁸)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰C(O)—, —S(O)₂—, or —NR¹⁰S(O)₂—; and n is 0 or 1, provided when

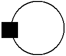

is substituted

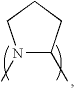

then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴ is optionally substituted aliphatic.

In another embodiment, the HCV protease inhibitor is a compound of structural Formula XXIV:

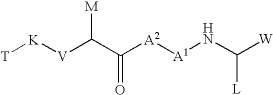

or a pharmaceutically acceptable salt, solvate or ester thereof;
wherein in Formula XXIV:

### W is:

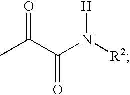

m is 0 or 1;

R² is hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl; wherein any R² carbon atom is optionally substituted with J;

J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;

J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoalkyl, halo, cyano, nitro, formyl, sulfonyl, or sulfonamido;

L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;

A¹ is a bond;

R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;

X is a bond, —C(H)(R⁷)—, —O—, —S—, or —N(R⁸)—;

R⁷ is hydrogen, alkyl, alkenyl, aryl, aralkyl, heterocyclyl, heterocyclylalkyl, heteroaryl, or heteroaralkyl, and is optionally substituted with 1-3 J groups;

R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R¹⁴, —SO₂R¹⁴, or carboxamido, and is optionally substituted with 1-3 J groups; or R⁸ and Z, together with the atoms to which they are bound, form a nitrogen containing mono- or bicyclic ring system optionally substituted with 1-3 J groups;

R¹⁴ is alkyl, aryl, aralkyl, heterocyclyl, heterocyclyalkyl, heteroaryl, or heteroaralkyl;

Y is a bond, —CH₂—, —C(O)—, —C(O)C(O)—, —S(O)—, —S(O)₂—, or —S(O)(NR⁷)—, wherein R⁷ is as defined above;

Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;

A² is a bond or

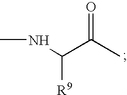

R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;

V is a bond, —CH₂—, —C(H)(R¹¹)—, —O—, —S—, or —N(R¹¹)—;

R¹¹ is hydrogen or C₁₋₃ alkyl;

K is a bond, —O—, —S—, —C(O)—, —S(O)—, —S(O)₂—, or —S(O)(NR¹¹)—, wherein R¹¹ is as defined above;

T is —R¹², -alkyl-R¹², -alkenyl-R¹², -alkynyl-R¹², —OR¹², —N(R¹²)₂, —C(O)R¹², —C(═NOalkyl)R¹², or

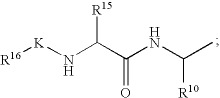

R¹² is hydrogen, aryl, heteroaryl, cycloalkyl, heterocyclyl, cycloalkyldienyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;

R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;

R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and

R¹⁶ is hydrogen, alkyl, aryl, heteroaryl, cycloalkyl, or heterocyclyl.

In another embodiment, the HCV protease inhibitor is a compound of structural Formula XXV:

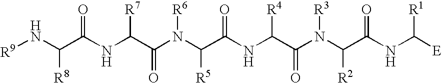

or a pharmaceutically acceptable salt, solvate or ester thereof;
wherein in Formula XXV:

E represents CHO or B(OH)₂;

R¹ represents lower alkyl, halo-lower alkyl, cyano-lower alkyl, lower alkylthio-lower alkyl, aryl-lower alkylthio-lower alkyl, aryl-lower alkyl, heteroaryl lower alkyl, lower alkenyl or lower alkynyl;

R² represents lower alkyl, hydroxy-lower alkyl, carboxy lower alkyl, aryl-lower alkyl, aminocarbonyl-lower alkyl or lower cycloalkyl-lower alkyl; and

R³ represents hydrogen or lower alkyl;

or R² and R³ together represent di- or trimethylene optionally substituted by hydroxy;

R⁴ represents lower alkyl, hydroxy-lower alkyl, lower cycloalkyl-lower alkyl, carboxy-lower alkyl, aryl lower alkyl, lower alkylthio-lower alkyl, cyano-lower alkylthio-lower alkyl, aryl-lower alkylthio-lower alkyl, lower alkenyl, aryl or lower cycloalkyl;

R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;

R⁶ represents hydrogen or lower alkyl;

R⁷ represent lower alkyl, hydroxy lower alkyl, carboxy lower alkyl, aryl-lower alkyl, lower cycloalkyl-lower alkyl or lower cycloalkyl;

R⁸ represents lower alkyl, hydroxy-lower alkyl, carboxy lower alkyl or aryl-lower alkyl; and

R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl.

In another embodiment, the HCV protease inhibitor is a compound of structural. Formula XXVI:

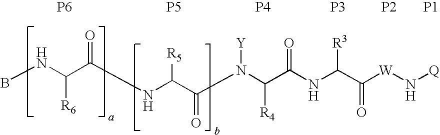

or a pharmaceutically acceptable salt, solvate or ester thereof;
wherein in Formula XXVI:

B is an acyl derivative of formula R₁₁—C(O)— wherein R₁₁ is C₁₋₁₀ alkyl optionally substituted with carboxyl; or R₁₁ is C₆ or C₁₀ aryl or C₇₋₁₆ aralkyl optionally substituted with a C₁₋₆ alkyl;

a is 0 or 1;

R₆, when present, is carboxy(lower)alkyl;

b is 0 or 1;

R₅, when present, is C₁₋₆ alkyl, or carboxy(lower)alkyl;

Y is H or C₁₋₆ alkyl;

R₄ is C₁₋₁₀ alkyl; C₃₋₁₀ cycloalkyl;

R₃ is C₁₋₁₀ alkyl; C₃₋₁₀ cycloalkyl;

W is a group of formula:

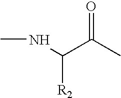

wherein R₂ is C₁₋₁₀ alkyl or C₃₋₇ cycloalkyl optionally substituted with carboxyl; C₆ or C₁₀ aryl; or C₇₋₁₆ aralkyl; or

W is a group of formula:

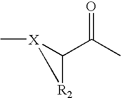

wherein X is CH or N; and

R₂′ is C₃₋₄ alkylene that joins X to form a 5- or 6-membered ring, said ring optionally substituted with OH; SH; NH₂; carboxyl; R₁₂; OR₁₂, SR₁₂, NHR₁₂ or NR₁₂R₁₂′ wherein R₁₂ and R₁₂′ are independently:

cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: O, S, and N; or

R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: O, S, and N;

said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl; C₆ or C₁₀ aryl, or heterocycle; said second ring optionally containing at least one heteroatom selected independently from the group consisting of: O, S, and N;

Q is a group of the formula:

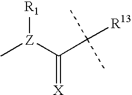

wherein Z is CH;

X is O or S;

R₁ is H, C₁₋₆ alkyl or C₁₋₆ alkenyl both optionally substituted with thio or halo;

and

R₁₃ is CO—NH—R₁₄ wherein R₁₄ is hydrogen, cyclic C₃₋₁₀ alkyl or acyclic C₁₋₁₀alkyl or cyclic C₃₋₁₀ alkenyl or acyclic C₂₋₁₀ alkenyl, said alkyl or alkenyl optionally substituted with NH₂, OH, SH, halo or carboxyl; said alkyl or alkenyl optionally containing at least one heteroatom selected independently from the group consisting of: 0, S, and N; or

R₁₄ is C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: O, S, and N;

said cyclic alkyl, cyclic alkenyl, aryl or aralkyl being optionally fused with a second 5-, 6-, or 7-membered ring to form a cyclic system or heterocycle; said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: O, S, and N;

with the proviso that when Z is CH, then R₁₃ is not an α-amino acid or an ester thereof;

Q is a phosphonate group of the formula:

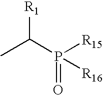

wherein R₁₅ and R₁₆ are independently C₆₋₂₀ aryloxy; and R₁ is as defined above.

In the above-shown structure of the compound of Formula XXVI, the terms P6, P5, P4, P3, P2 and P1 denote the respective amino acid moieties as is conventionally known to those skilled in the art. Thus, the actual structure of the compound of Formula XXVI is:

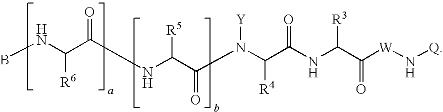

In another embodiment, the HCV protease inhibitor is a compound of structural Formula XXVII:

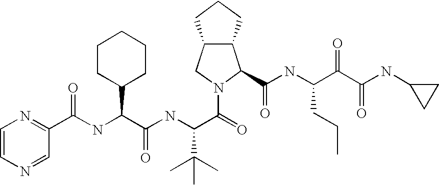

or a pharmaceutically acceptable salt, solvate or ester thereof.

In another embodiment, the HCV protease inhibitor is selected from the group consisting of:

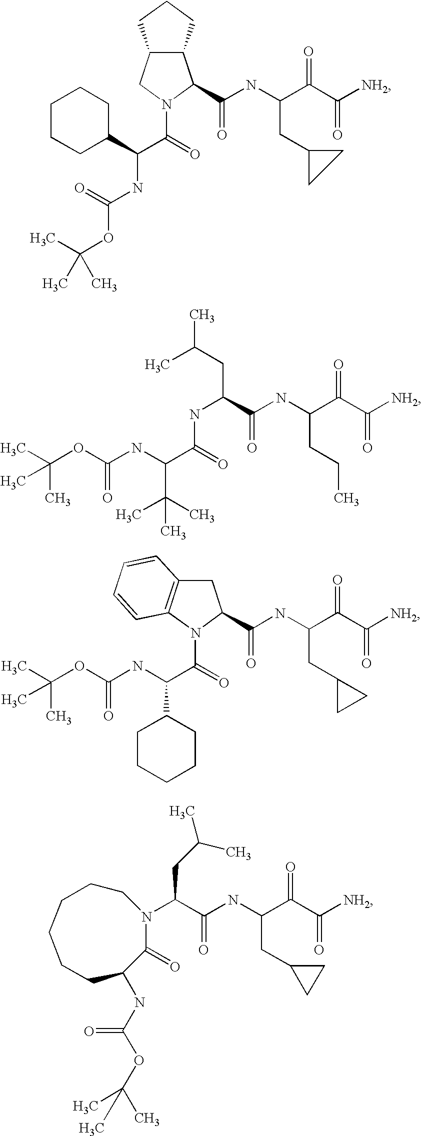

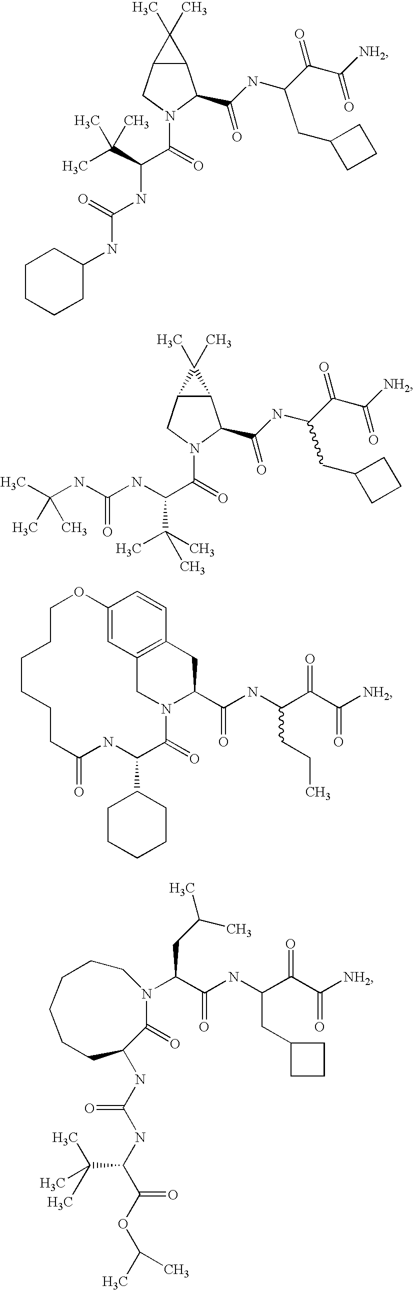

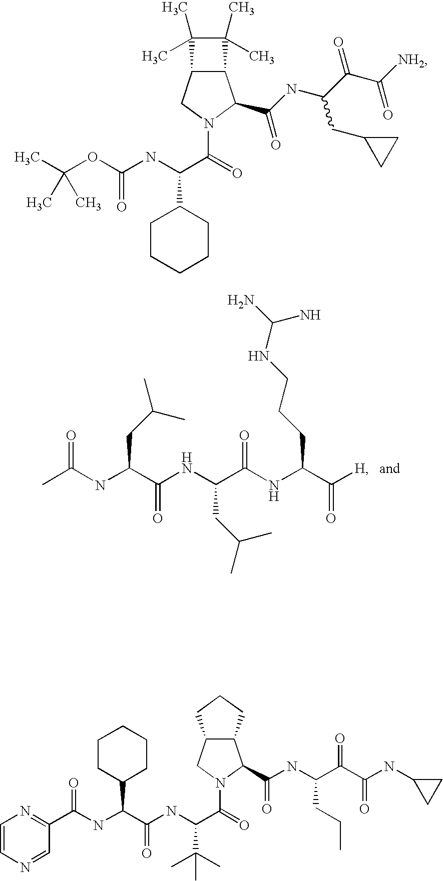

or a pharmaceutically acceptable salt, solvate or ester thereof.

### BRIEF DESCRIPTION OF THE DRAWINGS

The foregoing summary, as well as the following detailed description, will be better understood when read in conjunction with the appended drawings. In the drawings:

FIGS. 1A-1G depict inhibitors of CYP3A4, which are also inhibit HIV protease disclosed in U.S. Patent Publication No. US 2005/0209301 and U.S. Patent Publication No. US 2005/0267074.

FIG. 2 is a schematic of the clinical study conducted to evaluate the effect of ketoconazole and ibuprofen on the pharmacokinetics and metabolism of Formula I.

FIG. 3 depicts the mean plasma level (ng/ml) in human subjects of Formula Ia either alone or in combination with ketoconazole or ibuprofen over time.

FIG. 4 is a schematic of the clinical study to assess the pharmacokinetics, safety, and tolerability of Formula Ia administered in combination with ritonavir.

FIG. 5 depicts the mean plasma level (ng/ml) in human subjects of Formula Ia either alone or in combination with ritonavir over time.

FIG. 6 is a schematic of the proposed clinical study to assess the pharmacokinetics, safety, and tolerability of Formula XIVa in a rising multiple dose study as well as in a drug-drug interaction study when administered in combination with ritonavir.

### DETAILED DESCRIPTION OF THE INVENTION

The present invention provides medicaments, pharmaceutical compositions, pharmaceutical kits, and methods based on combinations comprising, separately or together: (a) at least one CYP3A4 inhibitor; and (b) at least one HCV protease inhibitor; for concurrent or consecutive administration in treating or ameliorating one or more symptoms of HCV or disorders associated with HCV in a subject in need thereof.

In one embodiment, the present invention provides medicaments, pharmaceutical compositions, pharmaceutical kits, and methods based on combinations comprising, separately or together: (a) at least one cytochrome P450 isoenzyme 3A4 (CYP3A4) inhibitor; and (b) at least one hepatitis C virus (HCV) protease inhibitor which is a compound of Formula I to XXVI below or a pharmaceutically acceptable salt, solvate or ester thereof; with the proviso that when at least one CYP3A4 inhibitor is ritonavir, then at least one HCV protease inhibitor is not Formula Ia; for concurrent or consecutive administration in treating or ameliorating one or more symptoms of HCV or disorders associated with HCV in a subject in need thereof.

In one preferred embodiment, the present invention provides medicaments and methods using the same comprising, separately or together: (a) at least one cytochrome P450 isoenzyme 3A4 (CYP3A4) inhibitor; and (b) at least one hepatitis C virus (HCV) protease inhibitor, wherein at least one HCV protease inhibitor is:

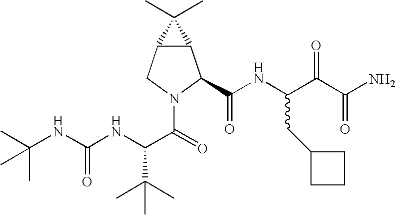

Formula Ia or a pharmaceutically acceptable salt, solvate or ester thereof; with the proviso that when at least one CYP3A4 inhibitor is ritonavir, then at least one HCV protease inhibitor is not Formula Ia; for concurrent or consecutive administration in treating or ameliorating one or more symptoms of HCV or disorders associated with HCV in a subject in need thereof.

In another preferred embodiment, the present invention provides medicaments and methods using the same comprising, separately or together: (a) at least one cytochrome P450 isoenzyme 3A4 (CYP3A4) inhibitor; and (b) at least one hepatitis C virus (HCV) protease inhibitor which is:

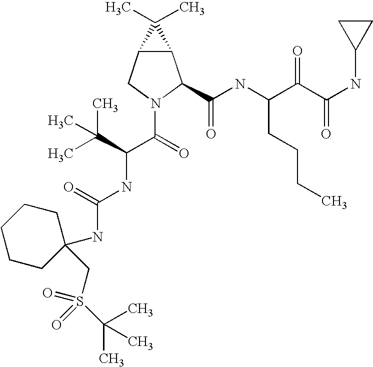

Formula XIVa or a pharmaceutically acceptable salt, solvate or ester thereof; for concurrent or consecutive administration in treating or ameliorating one or more symptoms of HCV or disorders associated with HCV in a subject in need thereof.

In yet another preferred embodiment, the present invention provides medicaments and methods using the same comprising, separately or together: (a) at least one cytochrome P450 isoenzyme 3A4 (CYP3A4) inhibitor; and (b) at least one hepatitis C virus (HCV) protease inhibitor which is:

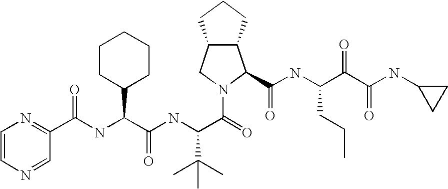

Formula XXVII or a pharmaceutically acceptable salt, solvate or ester thereof; for concurrent or consecutive administration in treating or ameliorating one or more symptoms of HCV or disorders associated with HCV in a subject in need thereof.

The present invention also provides medicaments, pharmaceutical compositions, pharmaceutical kits, and methods based on combinations comprising, separately or together: (a) at least one cytochrome P450 isozyme 3A4 (CYP3A4) inhibitor; and (b) at least one anti-HCV agent selected from the group consisting of a HCV protease inhibitor, a HCV polymerase inhibitor, a HCV NS3 helicase inhibitor, an inhibitor of HCV entry, an inhibitor of HCV p7, and a combination of two or more thereof; for concurrent or consecutive administration in treating or ameliorating one or more symptoms of HCV or disorders associated with HCV in a subject in need thereof.

In one embodiment, the present invention provides medicaments and methods using the same comprising, separately or together: (a) at least one cytochrome P450 isozyme 3A4 (CYP3A4) inhibitor; and (b) at least one anti-HCV agent which is a compound of Formula I to XXVI below or a pharmaceutically acceptable salt, solvate or ester thereof; with the proviso that when at least one CYP3A4 inhibitor is ritonavir then at least one anti-HCV agent is not Formula Ia; for concurrent or consecutive administration in treating or ameliorating one or more symptoms of HCV or disorders associated with HCV in a subject in need thereof.

In one embodiment, the present invention provides medicaments and methods using the same comprising, separately or together: (a) at least one cytochrome P450 isozyme 3A4 (CYP3A4) inhibitor; and (b) at least one anti-HCV agent which is:

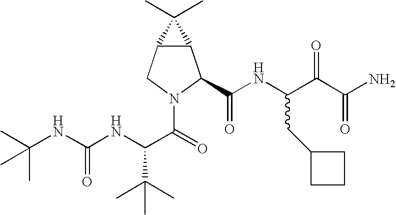

Formula Ia or a pharmaceutically acceptable salt, solvate or ester thereof; with the proviso that when at least one CYP3A4 inhibitor is ritonavir then at least one anti-HCV agent is not Formula Ia; for concurrent or consecutive administration in treating or ameliorating one or more symptoms of HCV or disorders associated with HCV in a subject in need thereof.

In one preferred embodiment, the present invention provides medicaments and methods using the same comprising, separately or together: (a) at least one cytochrome P450 isozyme 3A4 (CYP3A4) inhibitor; and (b) at least one anti-HCV agent which is:

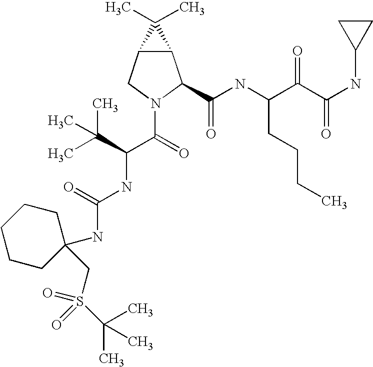

Formula XIVa or a pharmaceutically acceptable salt, solvate or ester thereof; for concurrent or consecutive administration in treating or ameliorating one or more symptoms of HCV or disorders associated with HCV in a subject in need thereof.

In yet another preferred embodiment, the present invention provides medicaments and methods using the same comprising, separately or together: (a) at least one cytochrome P450 isozyme 3A4 (CYP3A4) inhibitor; and (b) at least one anti-HCV agent which is:

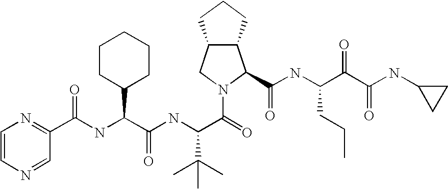

Formula XXVII or a pharmaceutically acceptable salt, solvate or ester thereof; for concurrent or consecutive administration in treating or ameliorating one or more symptoms of HCV or disorders associated with HCV in a subject in need thereof.

In one embodiment, the medicament further comprises at least one other therapeutic agent. In a preferred embodiment, at least one other therapeutic agent is an immunomodulatory agent that enhances an antiviral response such as an interferon or a toll-like receptor-7 (TLR-7) agonist. In one embodiment, wherein at least one other therapeutic agent is an interferon, the medicament further comprises ribavirin. In another preferred embodiment, at least one other therapeutic agent is ribavirin. In yet another preferred embodiment, at least one other therapeutic agent is interferon, ribavirin, levovirin, VP 50406, ISIS 14803, HEPTAZYME, VX 497, THYMOSIN, MAXAMINE, mycophenolate mofetil, or an interleukin-10 (IL-10) antagonist or an IL-10 receptor antagonist. In still another preferred embodiment, at least one other therapeutic agent is an antibody specific to IL-10. Preferably, the antibody specific to IL-10 is humanized 12G8.

In one embodiment, the interferon is a pegylated interferon. In another embodiment, the interferon is interferon-alpha, PEG-interferon alpha conjugates, interferon alpha fusion polypeptides, consensus interferon, or a mixture of two or more thereof. In yet another embodiment, the interferon is ROFERON™, PEGASYS™, INTRON™, PEG-INTRON™, BEROFOR ALPHA™, and INFERGEN™, or a mixture of two or more thereof.

### CYP3A4 Inhibitors

In one embodiment, at least one CYP3A4 inhibitor is selected from the group of CYP3A4 inhibitors referred to in the following documents (which are incorporated by reference herein): US20040052865A1, US20030150004A1, US20060099667A1, US20030096251A1, US20060073099A1, US20050272045A1, US20020061836A1, US20020016681A1, US20010041706A1, US20060009645A1, US20050222270A1, US20050031713A1, US20040254156A1, US20040214848A1, WO0173113A2, WO2005068611A1, US20050171037A1, WO2003089657A1, WO2003089656A1, WO2003042898A2, US20040243319A1, WO0045817A1, WO2006037993A2, WO2004021972A2, WO2006024414A2, WO2004060370A1, WO9948915A1, WO2006054755A1, WO2006037617A1, JP2006111597A, WO0111035A1, WO9844939A1, WO2003026573A2, WO2003047594A1, WO0245704A2, WO2005020962A1, WO2006021456A1, US20040047920A1, WO2003035074A1, WO2005007631A1, WO2005034963A1, WO2006061714A2, WO0158455A1, WO2003040121A1, WO2002094865A1, WO0044933A1, U.S. Pat. No. 6,673,778B1, WO2005098025A2, US20040106216A1, WO0017366A2, WO9905299A1, WO9719112A1, EP1158045A1, WO0034506A2, U.S. Pat. No. 5,886,157A, WO9841648A2, U.S. Pat. No. 6,200,754B1, U.S. Pat. No. 6,514,687B1, WO2005042020A2, WO9908676A1, WO9817667A1, WO0204660A2, WO2003046583A2, WO2003052123A1, WO2003046559A2, US20040101477A1, US20040084867A1, JP10204091A, WO9635415A2 WO9909976, WO98053658, US2004058982, U.S. Pat. No. 6,248,776, U.S. Pat. No. 6,063,809, U.S. Pat. No. 6,054,477, U.S. Pat. No. 6,162,479, WO2000054768, U.S. Pat. No. 6,309,687, U.S. Pat. No. 6,476,066, U.S. Pat. No. 6,660,766, WO 2004037827, U.S. Pat. No. 6,124,477, U.S. Pat. No. 5,820,915, U.S. Pat. No. 5,993,887, U.S. Pat. No. 5,990,154, U.S. Pat. No. 6,255,337, Fukuda et al., “Specific CYP3A4 inhibitors in grapefruit juice: furocoumarin dimers as components of drug interaction,” Pharmacogenetics, 7(5):391-396 (1997), Matsuda et. al., “Taurine modulates induction of cytochrome P450 3A4 mRNA by rifampicin in the HepG2 cell line,” Biochim Biophys Acta, 1593(1):98-98 (2002); Tassaneeyakul et al., “Inhibition selectively of grapefruit juice components on human cytochromes P450,” Arch Biochem Biophys, 378(2):356-363 (2000); Widmer and Haun, “Variation in furanocoumarin content and new furanocoumarin dimers in commercial grapefruit (Citrus paradise Macf.) juices,” Journal of Food Science, 70(4):C307-C312 (2005).

Non-limiting examples of suitable CYP3A4 inhibitors include ketoconazole (NIZORAL™, commercially available from Janssen Pharmaceutica), itraconazole (SPORANOX®, commercially available from Janssen-Cilag), ritonavir (NORVIR® commercially available from Abbott), nelfinavir (VIRACEPT® commercially available from Pfizer), indinavir (CRIXIVAN® commercially available from Merck & Co., Inc), erythromycin (AKNE-MYCIN®, A/T/S®, EMGEL®, ERYCETTE®, ERYDERM®, ERYGEL®, ERYMAX®, ERY-SOL®, ERYTHRA-DERM®, ETS®, STATICIN®, THERAMYCIN Z®, T-STAT®, ERYC®, ERY-TAB®, ERYTHROMYCIN BASE FILMTAB®, PCE® DISPERTAB®), clarithromycin (BIAXIN®), troleandomycin (TAO®), saquinavir, nefazodone, fluconazole, grapefruit juice, fluoxetine (PROZAC® commercially available from Eli Lilly and Company, Zoloft® commercially available from Pfizer Pharmaceuticals, ANAFRANIL® commercially available from Mallinckrodt Inc.), fluvoxamine (LUVOX®), Zyflo (ZILEUTON® commercially available from Abbott Laboratories), clotrimazole (FUNGOID® Solution, GYNE-LOTRIMIN®, GYNELOTRIMIN® 3, GYNE-LOTRIMIN® 3 Combination Pack, GYNE-LOTRIMIN®-3, LOTRIM® AF Jock Itch Cream, LOTRIMIN®, LOTRIMIN® AF, MYCELEX® TROCHE, MYCELEX®-7), midazolam (available from Apotex Corp.), naringenin, bergamottin, BAS 100 (available from Bioavailability Systems). In one preferred embodiment, the CYP3A4 inhibitor is ketoconazole (NIZORAL™) or clarithromycin (BIAXIN®). In another preferred embodiment, the CYP3A4 inhibitor is BAS 100 (available from Bioavailability Systems).

Preferably, the clarithromycin is administered at a unit dosage sufficient to increase the bioavailability of the HCV protease inhibitor. Preferably, the clarithromycin is administered at a unit dosage of about 5 mg to about 249 mg per day. Preferably, the clarithromycin is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 249 mg per day.

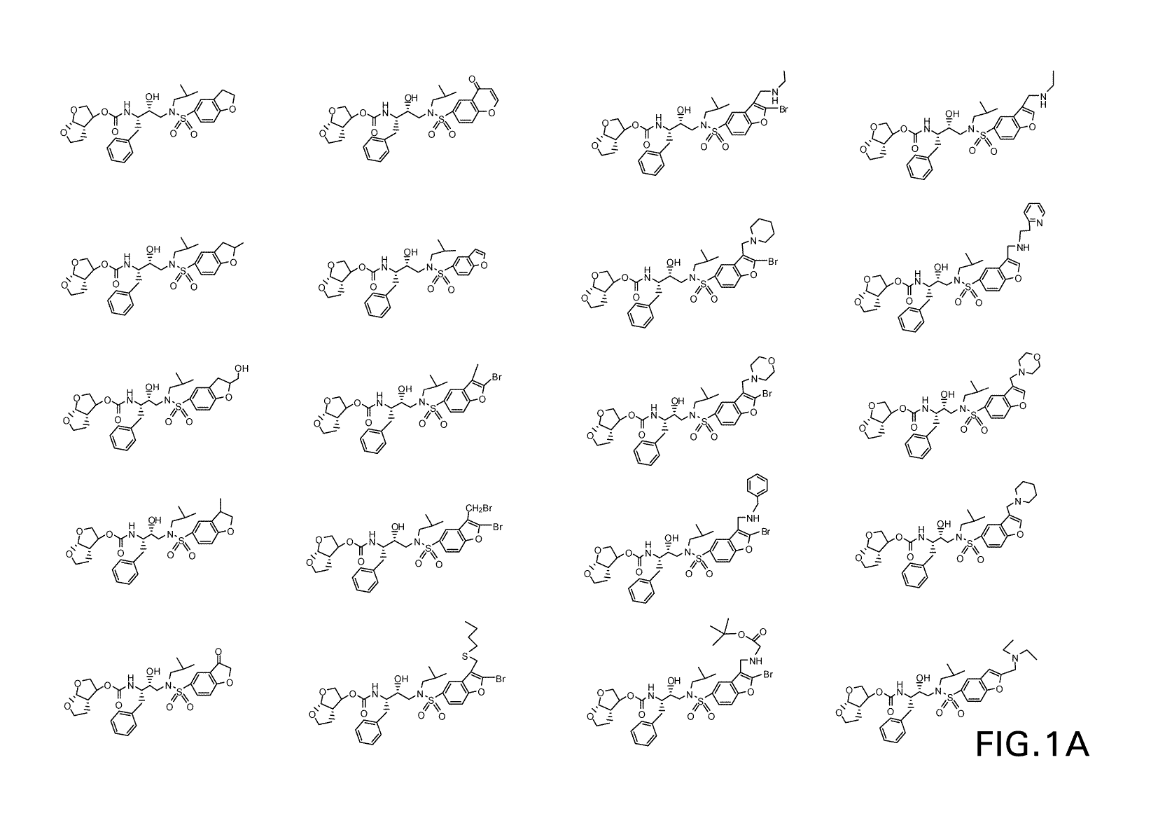

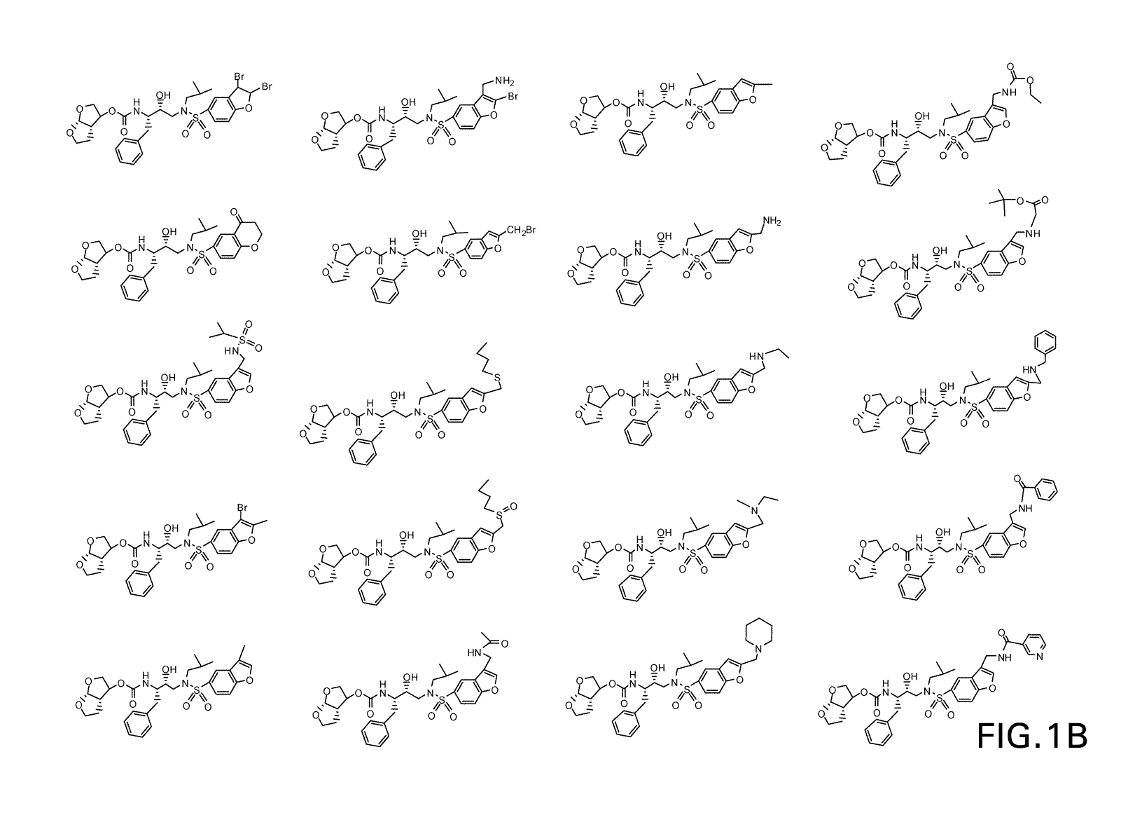

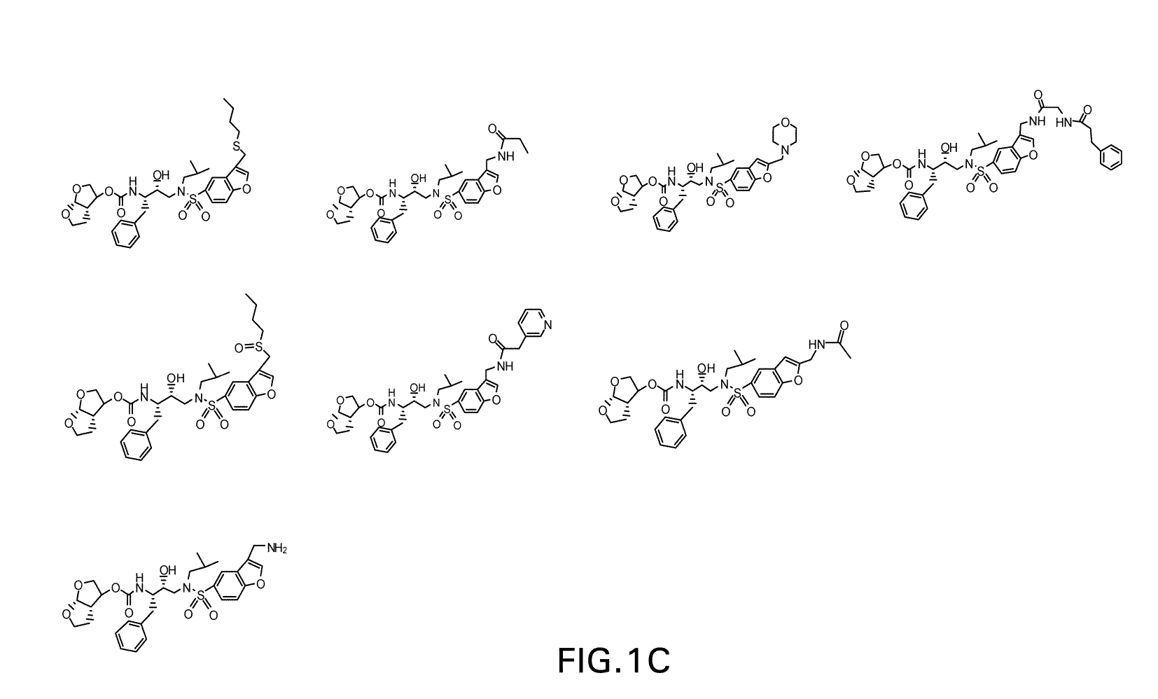

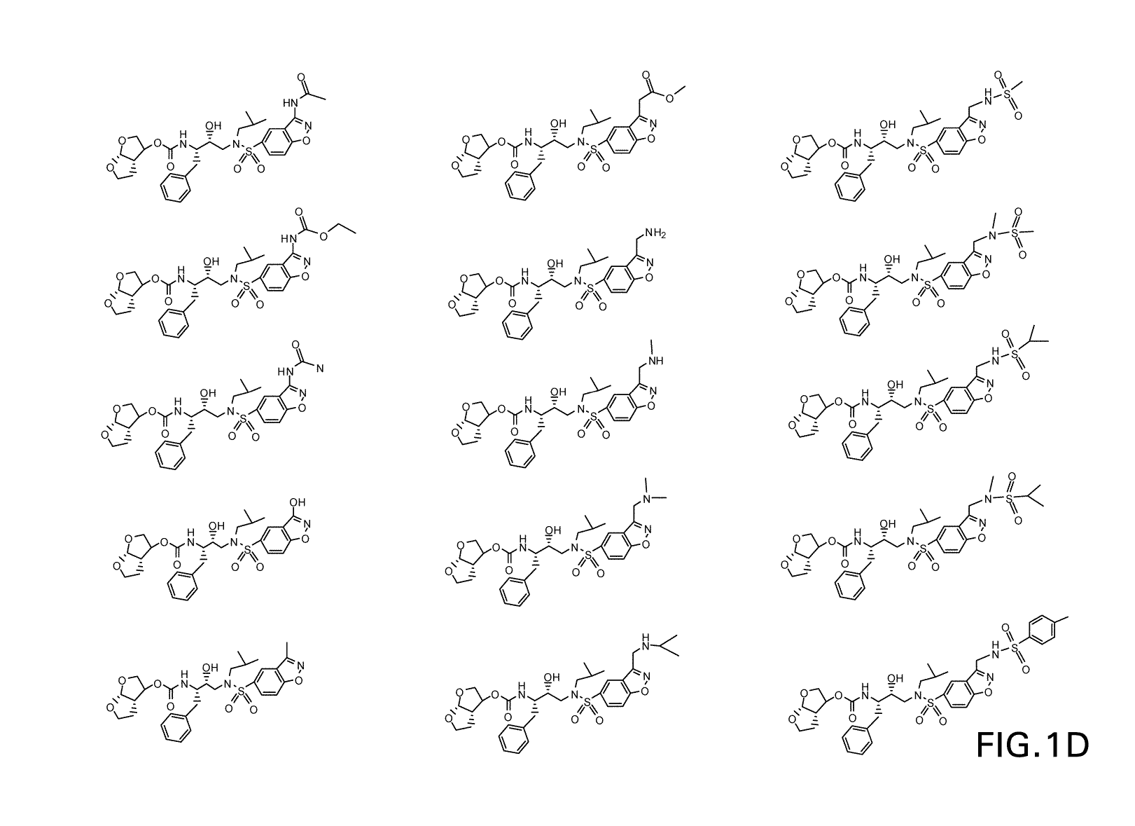

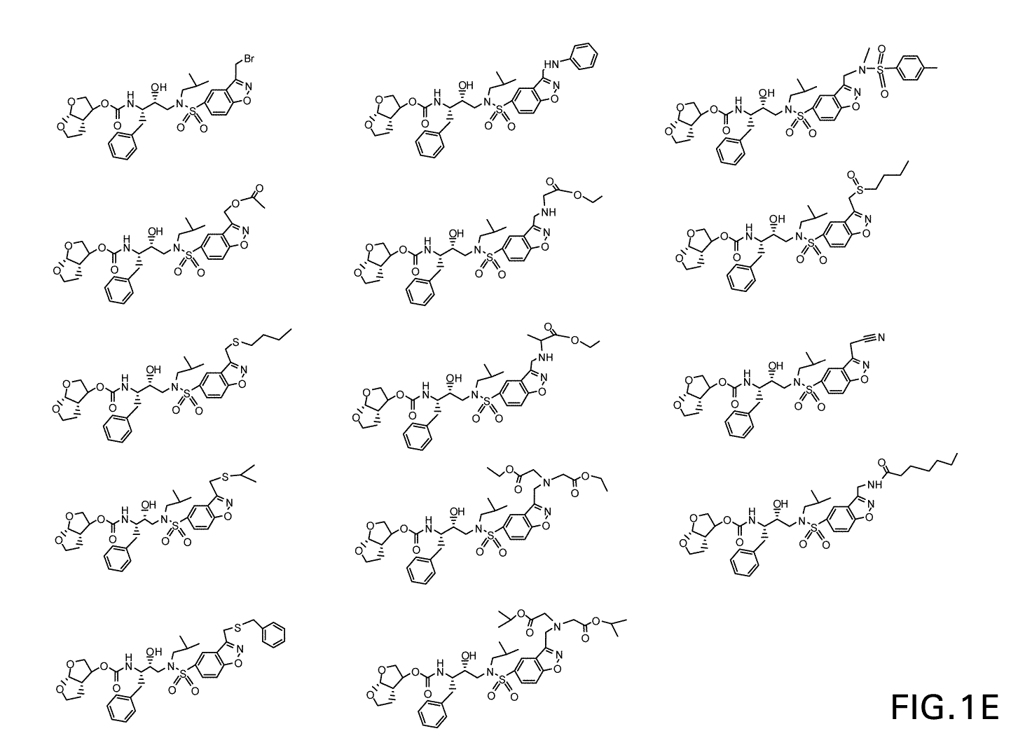

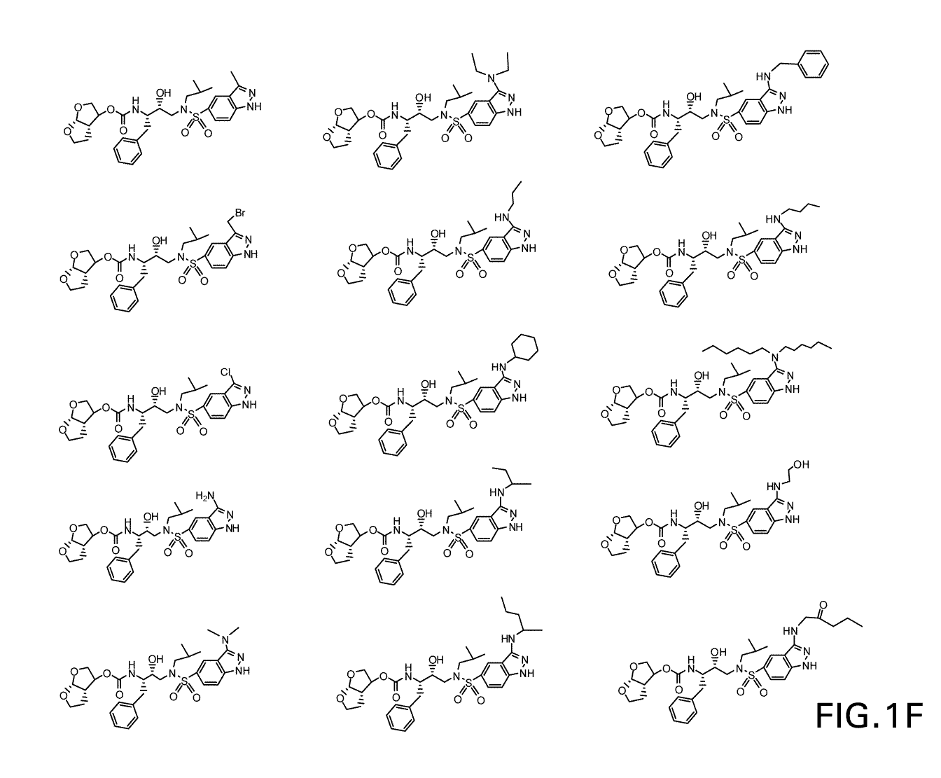

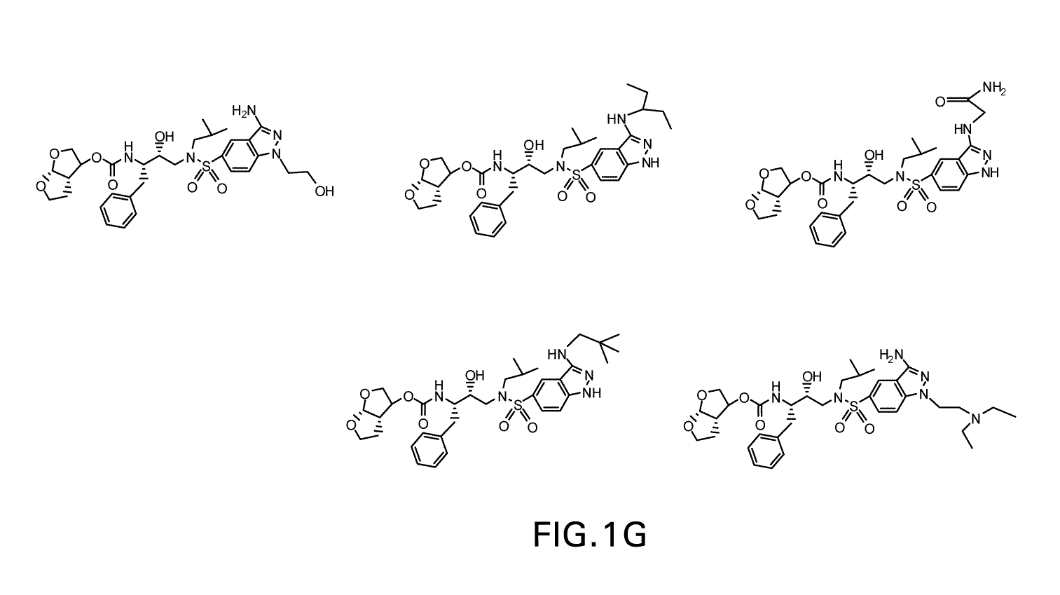

In addition, non-limiting examples of suitable compounds that inhibit HIV protease which have also been identified as CYP3A4 inhibitors are disclosed in US 2005/0209301 (at page 3, paragraph [0025] to page 5, paragraph [0071] and page 10, paragraph [0170] to page 12, paragraph [0226]) as well as US 2005/0267074 (at page 3, paragraph [0025], paragraph [0028] to page 7, paragraph [0114], page 7, paragraph to paragraph [0124], and FIGS. 1-3) incorporated herein by reference. The following is a list of specific compounds depicted in US 2005/0209301: {1-Benzyl-3-[(3-dimethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-dimethylamino-ethylidene)-2-oxo-2,3-dihydro-1H-1-indole-5-sulfonyl]-isobutyl-amino}-2-hydroxy-propyl)-carbamic acid hexahydro-furo[2,3-b]furan-3-yl ester; [1-Benzyl-3-({3-[(ethyl-methyl-amino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-3-({3-[1-(ethyl-methyl-amino)-ethylidene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(methyl-propyl-amino)-methylene-]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[1-(methyl-propyl-amino)-ethylidene]-2-oxo-2,3-dihydro-1H-indole-5-sulfonyl}-amino)-propyl]-carbamic acid hexahydro-furo[2,3-b]furan-3-yl ester; {1-Benzyl-3-[(3-diethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-diethylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; {1-Benzyl-3-[(3-dipropylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-dipropylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; {1-Benzyl-2-hydroxy-3-[isobutyl-(2-oxo-3-piperidin-1-ylmethylene-2,-3-dihydro-1H-indole-5-sulfonyl)amino]-propyl}-carbamic acid hexahydro-furo[2,3-b]furan-3-yl ester; (1-Benzyl-2-hydroxy-3-{isobutyl-[2-oxo-3-(1-piperidin-1-yl-ethylidene)-2,3-dihydro-1H-indole-5-sulfonyl]-amino}-propyl)-carbamic acid hexahydro-furo[2,3-b]furan-3-yl ester; {1-Benzyl-2-hydroxy-3-[isobutyl-(2-oxo-3-piperazin-1-ylmethylene-2,-3-dihydro-1H-indole-5-sulfonyl)-amino]-propyl}-carbamic acid hexahydro-furo[2,3-b]furan-3-yl ester; {1-Benzyl-2-hydroxy-3-[isobutyl-(3-morpholin-4-ylmethylene-2-oxo-2,-3-dihydro-1H-indole-5-sulfonyl)-amino]-propyl}-carbamic acid hexahydro-furo[2,3-b]furan-3-yl ester; {3-[(3-Aminomethylene-2-oxo-2,3-dihydro-1H-indole-5-sulfonyl)-isobutyl-amino]-1-benzyl-2-hydroxy-propyl}-carbamic acid hexahydro-furo[2,3-b]furan-3-yl ester; (3-{[3-(1-Amino-ethylidene)-2-oxo-2,3-dihydro-1H-indole-5-sulfonyl]-isobutyl-amino}-1-benzyl-2-hydroxy-propyl)-carbamic acid hexahydro-furo[2,3-b]furan-3-yl ester; {1-Benzyl-2-hydroxy-3-[isobutyl-(3-methylaminomethylene-2-oxo-2,3-dihydro-1H-indole-5-sulfonyl)-amino]-propyl}-carbamic acid hexahydro-furo[2,3-b]furan-3-yl ester; (1-Benzyl-2-hydroxy-3-{isobutyl-[3-(1-methylamino-ethylidene)-2-oxo-2,3-dihydro-1H-indole-5-sulfonyl]-amino}-propyl)-carbamic acid hexahydro-furo[2,3-b]furan-3-yl ester; {1-Benzyl-3-[(3-ethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-ethylamino-ethylidene)-2-oxo-2,3-dihydro-1H-indo-1e-5-sulfonyl]-isobutyl-amino}-2-hydroxy-propyl)-carbamic acid hexahydro-furo[2,3-b]furan-3-yl ester; [1-Benzyl-2-hydroxy-3-(isobutyl-{2-oxo-3-[(2,2,2-trifluoro-ethylamino)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[1-(2,2,2-trifluoro-ethylamino)-ethylidene]-2,3-dihydro-1H-indole-5-sulfonyl}-amino)-propyl]-carbamic acid hexahydro-furo[2,3-b]furan-3-yl ester; [1-Benzyl-2-hydroxy-3-({3-[(2-hydroxy-ethylamino)-methylene]-2-oxo-2,3-dihydro-1H-indole-5-sulfonyl}-isobutyl-amino)-propyl]-carbamic acid hexahydro-furo[2,3-b]furan-3-yl ester; [1-Benzyl-2-hydroxy-3-({3-[1-(2-hydroxy-ethylamino)-ethylidene]-2-oxo-2,3-dihydro-1H-indole-5-sulfonyl}-isobutyl-amino)-propyl]-carbamic acid hexahydro-furo[2,3-b]furan-3-yl ester; [1-Benzyl-2-hydroxy-3-(isobutyl-{3-[(2-methoxy-ethylamino)-methylene]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[1-(2-methoxy-ethylamino)-ethylidene]-2-oxo-2,3-dihydro-1H-indole-5-sulfonyl}-amino)-propyl]-carbamic acid hexahydro-furo[2,3-b]furan-3-yl ester; [1-Benzyl-3-({3-[(2-dimethylamino-ethylamino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-3-({3-[1-(2-dimethylamino-ethylamino)-ethylidene]-2-oxo-2-,3-dihydro-1H-indole-5-sulfonyl}-isobutyl-amino)-2-hydroxy-propyl]-carbamic acid hexahydro-furo[2,3-b]furan-3-yl ester; (1-Benzyl-2-hydroxy-3-{isobutyl-[3-(isopropylamino-methylene)-2-oxo-2,3-dihydro-1H-indole-5-sulfonyl]-amino}-propyl)-carbamic acid hexahydro-furo[2,3-b]furan-3-yl ester; (1-Benzyl-2-hydroxy-3-{isobutyl-[3-(1-isopropylamino-ethylidene)-2-oxo-2,3-dihydro-1H-indole-5-sulfonyl]-amino}-propyl)-carbamic acid hexahydro-furo[2,3-b]furan-3-yl ester; {1-Benzyl-2-hydroxy-3-[isobutyl-(2-oxo-3-propylaminomethylene-2,3-dihydro-1H-indole-5-sulfonyl)-amino]-propyl}-carbamic acid hexahydro-furo[2,3-b]furan-3-yl ester; (1-Benzyl-2-hydroxy-3-{isobutyl-[2-oxo-3-(1-propylamino-ethylidene)-2,3-dihydro-1H-indole-5-sulfonyl]-amino}-propyl)-carbamic acid hexahydro-furo[2,3-b]furan-3-yl ester; {1-Benzyl-2-hydroxy-3-[isobutyl-(2-oxo-3-pyrrolidin-2-ylidene-2,3-dihydro-1H-indole-5-sulfonyl)-amino]-propyl}-carbamic acid hexahydro-furo[2,3-b]furan-3-yl ester; {1-Benzyl-3-[(3-butylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-butylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; (1-Benzyl-2-hydroxy-3-{isobutyl-[3-(isobutylamino-methylene)-2-oxo-2,3-dihydro-1H-indole-5-sulfonyl]-amino}-propyl)-carbamic acid hexahydro-furo[2,3-b]furan-3-yl ester; (1-Benzyl-2-hydroxy-3-{isobutyl-[3-(1-isobutylamino-ethylidene)-2-oxo-2,3-dihydro-1H-indole-5-sulfonyl]-amino}-propyl)-carbamic acid hexahydro-furo[2,3-b]furan-3-yl ester; (1-Benzyl-3-{[3-(tert-butylamino-methylene)-2-oxo-2,3-dihydro-1H-indole-5-sulfonyl]-isobutyl-amino}-2-hydroxy-propyl)-carbamic acid hexahydro-furo[2,3-b]furan-3-yl ester; (1-Benzyl-3-{[3-(1-tert-butylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; [1-Benzyl-3-({3-[(2,2-dimethyl-propylamino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-3-({3-[1-(2,2-dimethyl-propylamino)-ethylidene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(2-methyl-butylamino)-methylene-]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[(3-methyl-butylamino)-methylene-]-2-oxo-2,3-dihydro-1H-indole-5-sulfonyl}-amino)-propyl]-carbamic acid hexahydro-furo[2,3-b]furan-3-yl ester; [1-Benzyl-3-({3-[(3,3-dimethyl-butylamino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(1-isopropyl-2-methyl-propylamino)-methylene]-2-oxo-2,3-dihydro-1H-indole-5-sulfonyl}-amino)-propyl]-carbamic acid hexahydro-furo[2,3-b]furan-3-yl ester; {1-Benzyl-2-hydroxy-3-[isobutyl-(2-oxo-3-phenylaminomethylene-2,3-dihydro-1H-indole-5-sulfonyl)-amino]-propyl}-carbamic acid hexahydro-furo[2,3-b]furan-3-yl ester; (1-Benzyl-3-{([3-(benzylamino-methylene)-2-oxo-2,3-dihydro-1H-indole-5-sulfonyl]-isobutyl-amino}-2-hydroxy-propyl)-carbamic acid hexahydro-furo[2,3-b]furan-3-yl ester; (1-Benzyl-3-{[3-(1-benzylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; [1-Benzyl-3-({3-[(cyclohexylmethyl-amino)-methylene]-2-oxo-2,3-dihydro-1H-indole-5-sulfonyl]-isobutyl-amino)-2-hydroxy-propyl}-carbamic acid hexahydro-furo[2,3-b]furan-3-yl ester; {1-Benzyl-2-hydroxy-3-[isobutyl-(2-oxo-3-{[(pyridin-4-ylmethyl)-amino]-methylene}-2,3-dihydro-1H-indole-5-sulfonyl)-amino]-propyl}-carbamic acid hexahydro-furo[2,3-b]furan-3-yl ester; (1-Benzyl-2-hydroxy-3-{isobutyl-[2-oxo-3-(phenethylamino-methylene)-2,3-dihydro-1H-indole-5-sulfonyl]-amino}-propyl)-carbamic acid hexahydro-furo[2,3-b]furan-3-yl ester; [1-Benzyl-3-({3-[(2-cyclohex-1-enyl-ethylamino)-methylene]-2-oxo-2,-3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{2-oxo-3-[(2-pyridin-2-yl-ethylamino)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[(2-phenyl-propylamino)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[(4-phenyl-butylamino)-methylene]-2,3-dihydro-1H-indole-5-sulfonyl}-amino)-propyl]-carbamic acid hexahydro-furo[2,3-b]furan-3-yl ester; {1-Benzyl-2-hydroxy-3-[isobutyl-(3-nonylaminomethylene-2-oxo-2,3-dihydro-1H-indole-5-sulfonyl)-amino]-propyl}-carbamic acid hexahydro-furo[2,3-b]furan-3-yl ester; and (1-Benzyl-2-hydroxy-3-{[3-(1-hydroxy-ethylidene)-2-oxo-2,3-dihydro-1H-indole-5-sulfonyl]-isobutyl-amino}-propyl)-carbamic acid hexahydro-furo[2,3-b]furan-3-yl ester; and the pharmaceutically acceptable salts thereof, as single stereoisomers or mixtures of stereoisomers. Likewise, see FIGS. 1A-1G for a list of specific compounds depicted in US 2005/0267074. Notably, US 2005/0267074 emphasizes that compounds having a benzofuran moiety are potent inhibitors of CYP3A4. HIV inhibitors useful as CYP3A4 inhibitors are also disclosed in U.S. Ser. No. 60/785,761, filed Mar. 23, 2006, incorporated herein by reference.

In one embodiment, at least one CYP3A4 inhibitor is selected from the compounds disclosed in one or more of the following patent applications assigned to Sequoia Pharmaceuticals, Inc., the disclosure of each of which is incorporated herein by reference: U.S. Patent Publication No. US 2005/0209301 and U.S. Patent Publication No. US 2005/0267074.

In one embodiment, at least one CYP3A4 inhibitor is selected from the compounds disclosed in one or more of the following patents and patent applications assigned to Bioavailability Systems, LLC, the disclosure of each of which is incorporated herein by reference: US 2004058982, U.S. Pat. No. 6,248,776, U.S. Pat. No. 6,063,809, U.S. Pat. No. 6,054,477, U.S. Pat. No. 6,162,479, WO 2000054768, U.S. Pat. No. 6,309,687, U.S. Pat. No. 6,476,066, U.S. Pat. No. 6,660,766, WO 2004037827, U.S. Pat. No. 6,124,477, U.S. Pat. No. 5,820,915, U.S. Pat. No. 5,993,887, U.S. Pat. No. 5,990,154, U.S. Pat. No. 6,255,337. In particular, see, U.S. Pat. No. 6,063,809, column 5, line 30 to column 12, line 65; WO 2000054768, page 10, line 11 to page 22, line 1, and WO 2004037827, page 4 to page 17, incorporated herein by reference.

According to certain preferred embodiments of the present invention, at least one CYP3A4 inhibitor is ritonavir, ketoconazole, clarithromycin, BAS 100, a compound disclosed in U.S. Patent Publication No. US 2005/0209301 or U.S. Patent Publication No. US 2005/0267074, or a pharmaceutically acceptable salt, solvate or ester thereof. In one embodiment, at least one CYP3A4 inhibitor is ritonavir or a pharmaceutically acceptable salt, solvate or ester thereof. In another embodiment, at least one CYP3A4 inhibitor is ketoconazole or a pharmaceutically acceptable salt, solvate or ester thereof. In another embodiment, at least one CYP3A4 inhibitor is clarithromycin or a pharmaceutically acceptable salt, solvate or ester thereof. In another embodiment, at least one CYP3A4 inhibitor is a compound disclosed in U.S. Patent Publication No. US 2005/0209301 or U.S. Patent Publication No. US 2005/0267074 or a pharmaceutically acceptable salt, solvate or ester thereof. In another embodiment, at least one CYP3A4 inhibitor is BAS 100 or a pharmaceutically acceptable salt, solvate or ester thereof. Notably, at least one CYP3A4 inhibitor is identified by the Chemical Abstracts Services (CAS) Number 684217-04-7 which corresponds to the Chemical Abstract index name 7H-Furo[3,2-g][1]benzopyran-7-one, 4-[[(2E)-5-[(4R)-4′-[[(2E)-3,7-dimethyl-2,6-octadienyl]oxy]-5,5-dimethylspiro[1,3-dioxolane-2,7′-[7H]furo[3,2-g][1]benzopyran]-4-yl]-3-methyl-2-pentenyl]oxy]; the CAS Number 684217-03-6 which corresponds to the Chemical Abstract index name 7H-Furo[3,2-g][1]benzopyran-7-one, 4-[[(2E)-5-[(4R)-4′-[[2E)-6,7-dihydroxy-3,7-dimethyl-2-octenyl]oxy]-5,5-dimethylspiro[1,3-dioxolane-2,7′-[7H]furo[3,2-g][1]benzopyran]-4-yl]-3-methyl-2-pentenyl]oxy], or the CAS Number 267428-36-4 which corresponds to the Chemical Abstract index name 7H-Furo[3,2-g][1]benzopyran-7-one, 4-[[(2E)-5-[(2R,4R)-4′-[[(2E,6R)-6,7-dihydroxy-3,7-dimethyl-2-octenyl]oxy]-5,5-dimethylspiro[1,3-dioxolane-2,7′-[7H]furo[3,2-g][1]benzopyran]-4-yl]-3-methyl-2-pentenyl]oxy]; all of which is further described in WO 2004037827. In one embodiment, at least one CYP3A4 inhibitor has the structure shown below:

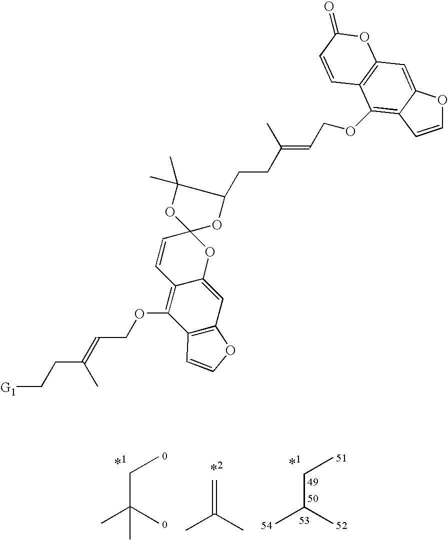

An effective amount of CYP3A4 inhibitor is an amount effective to increase the bioavailability of at least one HCV protease inhibitor. For any CYP3A4 inhibitor, the effective amount can be estimated initially either in cell culture assays or in a relevant animal model, such as monkey. The animal model may also be used to determine the appropriate concentration range and route of administration. Such information can be then be used to determine useful doses and routes for administration in humans.

### HCV Protease Inhibitors:

In one embodiment, at least one HCV protease inhibitor is selected from the group of HCV protease inhibitors referred to in the following documents (which are incorporated by reference herein): US20040048802A1, US20040043949A1, US20040001853A1, US20030008828A1, US20020182227A1, US20020177725A1, US20020150947A1, US20050267018A1, US20020034732A1, US20010034019A1, US20050153877A1, US20050074465A1, US20050053921 A1, US20040253577A1, US20040229936A1, US20040229840A1, US20040077551A1, EP1408031A1, WO9837180A2, U.S. Pat. No. 6,696,281B1, JP11137252A, WO0111089A1, U.S. Pat. No. 6,280,940B1, EP1106702A1, US20050118603A1, JP2000007645A, WO0053740A1, WO0020400A1, WO2004013349A2, WO2005027871 A2, WO2002100900A2, WO0155703A1, US20030125541A1, US20040039187A1, U.S. Pat. No. 6,608,027B1, US20030224977A1, WO2003010141 A2, WO2003007945A1, WO2002052015A2, WO0248375A2, WO0066623A2, WO0009543A2, WO9907734A2, U.S. Pat. No. 6,767,991B1, US20030187018A1, US20030186895A1, WO2004087741A1, WO2004039970A1, WO2004039833A1, WO2004037855A1, WO2004030670A1, US20040229818A1, US20040224900A1, WO2005028501A1, WO2004103996A1, WO2004065367A1, WO2004064925A1, WO2004093915A1, WO2004009121 A1, WO2003066103A1, WO2005034850A2, WO2004094452A2, WO2004015131A2, WO2003099316A1, WO2003099274A1, WO2003053349A2, WO2002060926A2, WO0040745A1, US658661561, WO2002061048A2, WO02481.57A2, WO0248116A2, WO2005017125A2, WO0022160A1, US20060051745A1, WO2004021871A2, WO2004011647A1, WO9816657A1, U.S. Pat. No. 5,371,017A, WO9849190A2, U.S. Pat. No. 5,807,829A, WO0005243A2, WO0208251 A2, WO2005067437A2, WO9918856A1, WO0004914A1, WO0212543A2, WO9845040A1, WO0140262A1, WO0102424A2, WO0196540A2, WO0164678A2, U.S. Pat. No. 5,512,391A, WO0218369A2, WO9846597A1, WO2005010029A1, WO2004113365A2, WO2004093798A2, WO2004072243A2, WO9822496A2, WO2004046159A1, JP11199509A, WO2005012288A1, WO2004108687A2, WO9740168A1, US20060110755A1, WO2002093519A2, US618790561, WO2003077729A2, WO9524414A1, WO2005009418A2, WO2004003000A2, US20050037018A1, WO9963998A1, WO0063444A2, WO9938888A2, WO9964442A1, WO0031129A1, WO0168818A2, WO9812308A1, WO9522985A1, WO0132691A1, WO9708304A2, WO2002079234A1, JP10298151A, JP09206076A, JP09009961A, JP2001103993A, JP11127861A, JP11124400A, JP11124398A, WO2003051910A2, WO2004021861A2, WO9800548A1, WO2004026896A2, WO0116379A1, U.S. Pat. No. 5,861,297A, WO2004007512A2, WO2004003138A2, WO2002057287A2, WO2004009020A2, WO2004000858A2, WO2003105770A2, WO0114517A1, WO9805333A1, U.S. Pat. No. 6,280,728B1, EP1443116A1, US20040063911A1, WO2003076466A1, WO2002087500A2, WO0190121A2, WO2004016222A2, WO9839030A1, WO9846630A1, WO0123331A1, WO9824766A1, U.S. Pat. No. 6,168,942B1, WO0188113A2, WO2005018330A1, WO2005003147A2, WO9115596A1, WO9719103A1, WO9708194A1, WO2002055693A2, WO2005030796A1, WO2005021584A2, WO2004113295A1, WO2004113294A1, WO2004113272A1, WO2003062228A1, WO0248172A2, WO0208198A2, WO0181325A2, WO0177113A2, WO0158929A1, WO9928482A2, WO9743310A1, WO9636702A2, WO9635806A1, WO9635717A2, U.S. Pat. No. 6,326,137B1, U.S. Pat. No. 6,251,583B1, U.S. Pat. No. 5,990,276A, U.S. Pat. No. 5,759,795A, U.S. Pat. No. 5,714,371A, U.S. Pat. No. 6,524,589B1, WO0208256A2, WO0208187A1, WO2003062265A2, U.S. Pat. No. 7,012,066B2, JP07184648A, JP06315377A, WO2002100851A2, WO2002100846A1, WO0039348A1, JP06319583A, JP11292840A, JP08205893A, WO0075338A2, WO0075337A1, WO2003059384A1, WO2002063035A2, WO2002070752A1, U.S. Pat. No. 6,190,920B1, WO2002068933A2, WO0122984A1, JP04320693A, JP2003064094A, WO0179849A2, WO0006710A1, WO0001718A2, WO0238799A2, WO2005037860A2, WO2005035525A2, WO2005025517A2, WO2005007681A2, WO2003035060A1, WO2003006490A1, WO0174768A2, WO0107027A2, WO0024725A1, WO0012727A1, WO9950230A1, WO9909148A1, WO9817679A1, WO9811134A1, WO9634976A1, WO2003087092A2, WO2005028502A1, WO 2004/052885 A1, U.S. Pat. No. 5,837,464A, DE20201549U1, WO2003090674A2, WO9727334A1, WO0034308A2, U.S. Pat. No. 6,127,116A, US20030054000A1, JP2001019699A, U.S. Pat. No. 6,596,545B1, U.S. Pat. No. 6,329,209B1, IT1299179, CA2370400, KR2002007244, KR165708, KR2000074387, KR2000033010, KR2000033011, KR2001107178, KR2001107179, ES2143918, KR2002014283, KR149198, KR2001068676, U.S. Pat. No. 6,846,802B2, US20040254117A1, U.S. Pat. No. 6,838,466B2, US20060025441 A9.

In one embodiment, at least one HCV protease inhibitor is selected from the group consisting of compounds of Formula I to XXVI detailed above or a pharmaceutically acceptable salt, solvate or ester thereof.

In one embodiment, at least one HCV protease inhibitor or anti-HCV agent which is selected from Formula Ito XXVII or pharmaceutically acceptable salts, solvates, or esters thereof is formulated as a pharmaceutical formulation described in U.S. Provisional Patent Application 60/873,872 filed Dec. 7, 2006, U.S. Provisional Patent Application 60/873,877 filed Dec. 7, 2006; U.S. Provisional Patent Application 60/873,928 filed Dec. 7, 2006; or U.S. patent application Ser. No. 11/636,701 filed Dec. 7, 2006.

In certain embodiments, when at least one CYP3A4 inhibitor is ritonavir, then at least one HCV protease inhibitor is not Formula Ia.

In one embodiment, at least one HCV protease inhibitor is selected from the group consisting of:

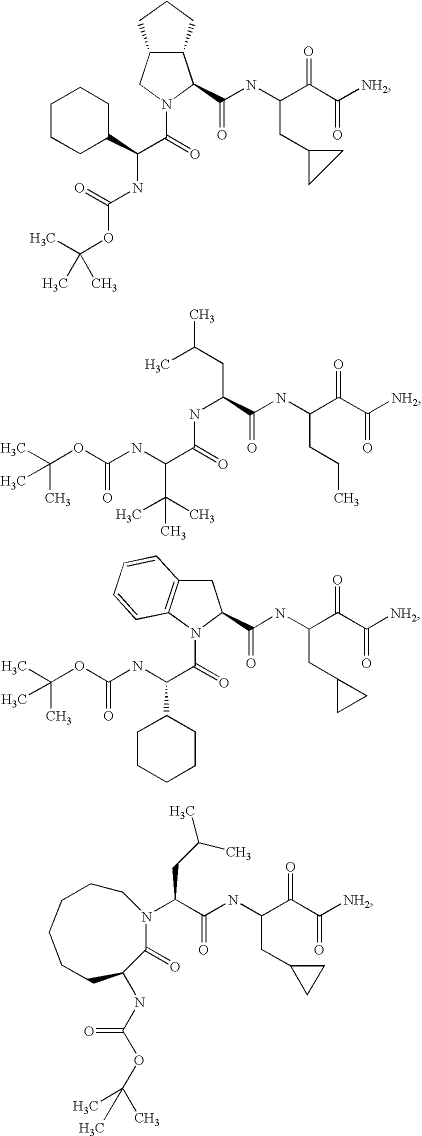

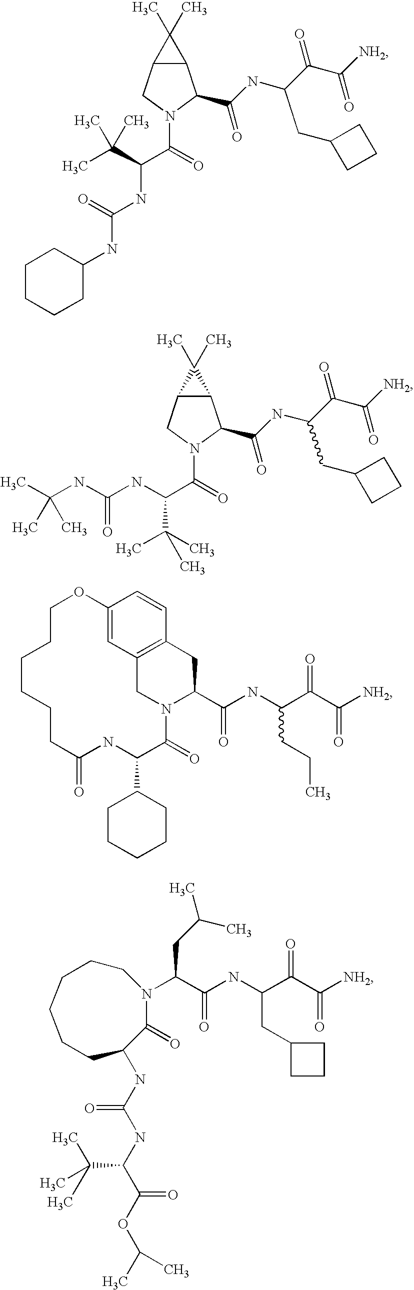

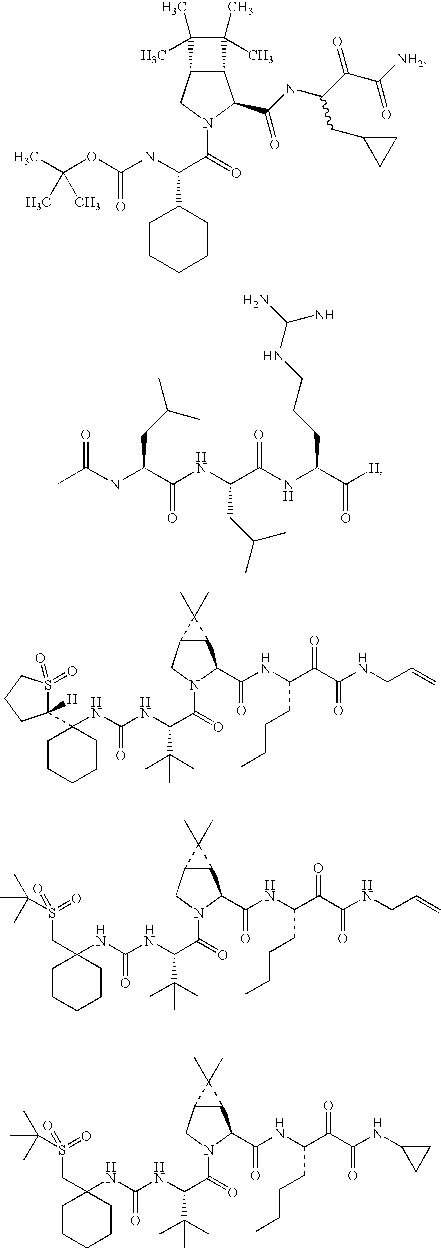

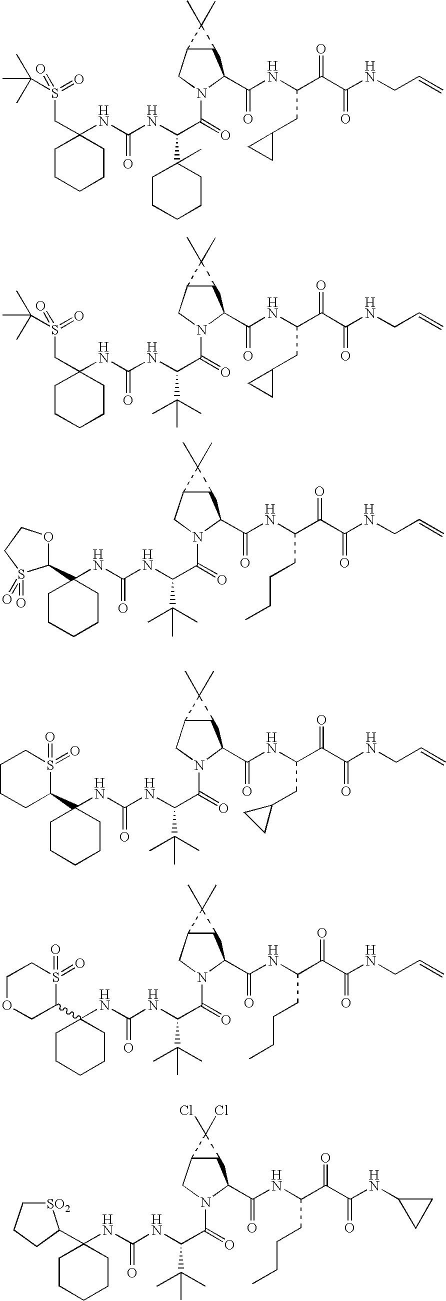

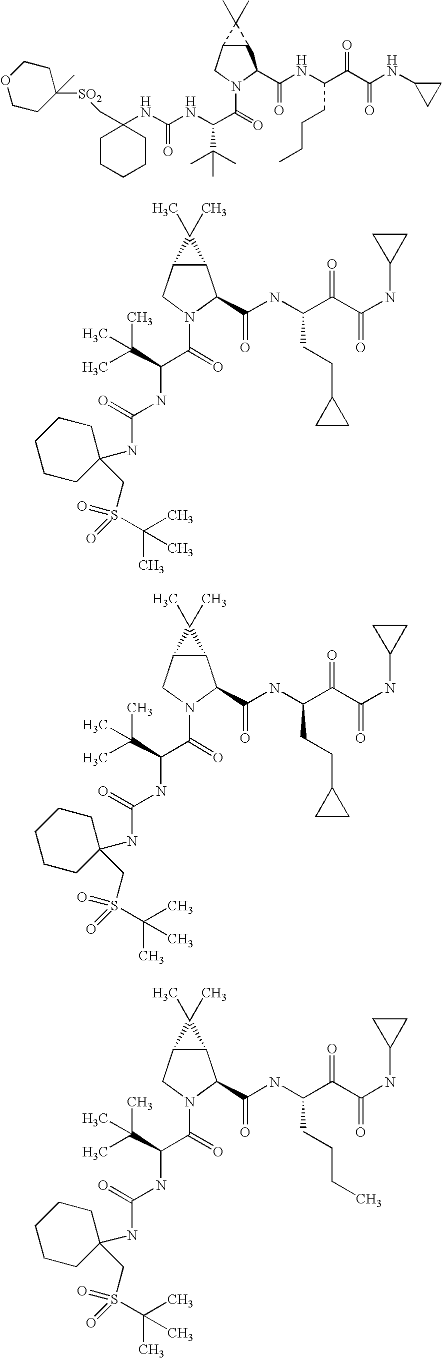

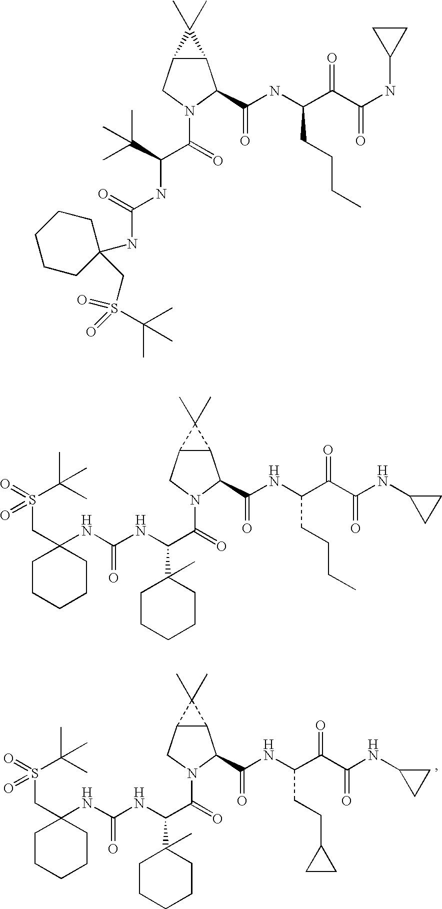

or a pharmaceutically acceptable salt, solvate or ester thereof.

In a preferred embodiment, at least one HCV protease inhibitor is a compound of Formula I, Formula XIV, or a pharmaceutically acceptable salt, solvate or ester thereof.

In one preferred embodiment, at least one HCV protease inhibitor is administered at a dosage range of about 100 to about 3600 mg per day (e.g., 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg; 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg per day). In one preferred embodiment, at least one HCV protease inhibitor is administered at a dosage range of about 400 mg to about 2500 mg per day. Note that the dosage of HCV protease inhibitor may be administered as a single dose (i.e., QD) or divided over 2-4 doses (i.e., BID, TID, or QID) per day. Preferably, at least one HCV protease inhibitor is administered orally.

In one embodiment, where at least one HCV protease inhibitor is a compound of Formula 1a, 1b, or Ic, or a pharmaceutically acceptable salt, solvate, or ester thereof, the preferred dosage range is about 400 mg to 2400 mg per day. In one preferred embodiment, where at least one HCV protease inhibitor is a compound of Formula 1a, 1b, or Ic, or a pharmaceutically acceptable salt, solvate, or ester thereof, the dosage is about 1200 mg per day administered as about 400 mg TID. In another preferred embodiment, where at least one HCV protease inhibitor is a compound of Formula 1a, 1b, or Ic, or a pharmaceutically acceptable salt, solvate, or ester thereof, the dosage is about 800 mg, 1600 mg, or 2400 mg per day administered as about 800 mg QD, BID, or TID, respectively.

In another embodiment, where at least one HCV protease inhibitor is a compound of Formula XIV, or a pharmaceutically acceptable salt, solvate, or ester thereof, the preferred dosage range is about 1350 mg to about 2500 mg per day. In one preferred embodiment, where at least one HCV protease inhibitor is a compound of Formula XIV, or a pharmaceutically acceptable salt, solvate, or ester thereof, the dosage is about 1350 mg, about 2250 mg, or about 2500 mg per day administered as about 450 mg TID, about 750 BID, or about 1250 BID, respectively.

In another embodiment, where at least one HCV protease inhibitor is Formula XXVII, or a pharmaceutically acceptable salt, solvate, or ester thereof, the preferred dosage range is about 1350 mg to about 2500 mg per day. In one preferred embodiment, where at least one HCV protease inhibitor is Formula XXVII, or a pharmaceutically acceptable salt, solvate, or ester thereof, the dosage is about 1350 mg, about 2250 mg, or about 2500 mg per day administered as about 450 mg TID, about 750 BID, or about 1250 BID, respectively.

Non-limiting examples of suitable HCV protease inhibitors of Formula I and methods of making the same are disclosed in WO 2003/062265 at page 48 through page 75, incorporated herein by reference.

In one embodiment, at least one HCV protease inhibitor is:

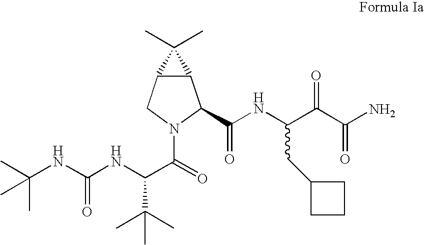

or a pharmaceutically acceptable salt, solvate or ester thereof, disclosed in U.S. Pat. No. 7,012,066 as Example XXIV, on columns 448-451, which is incorporated herein by reference.

The compound of Formula Ia has been separated into its isomer/diastereomers of Formulas Ib and Ic, as disclosed in US2005/0249702 published Nov. 10, 2005. In one embodiment, at least one HCV protease inhibitor is:

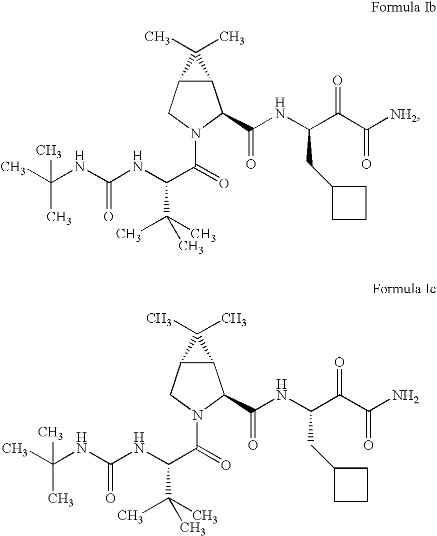

or a pharmaceutically acceptable salt, solvate, or ester thereof.
The chemical name of the compound of Formula Ic is (1R,2S,5S)—N-[(1S)-3-amino-1-(cyclobutylmethyl)-2,3-dioxopropyl]-3-[(2S)-2-[[[(1,1-dimethylethyl)amino]carbonyl]amino]-3,3-dimethyl-1-oxobutyl]-6,6-dimethyl-3-azabicyclo[3.1.0]hexane-2-carboxamide.

Processes for making compounds of Formula I are disclosed in U.S. Patent Publication Nos. 2005/0059648, 2005/0020689 and 2005/0059800, incorporated by reference herein.

Non-limiting examples of suitable compounds of Formula II and methods of making the same are disclosed in WO02/08256 and in U.S. Pat. No. 6,800,434, at col. 5 through col. 247, incorporated herein by reference.

Non-limiting examples of suitable compounds of Formula III and methods of making the same are disclosed in International Patent Publication WO02/08187 and in U.S. Patent Publication 2002/0160962 at page 3, paragraph 22 through page 132, incorporated herein by reference.

Non-limiting examples of suitable compounds of Formula IV and methods of making the same are disclosed in U.S. Pat. No. 6,894,072, granted May 17, 2005, col. 5, lines 54 through col. 49, line 48, at incorporated herein by reference.

Non-limiting examples of suitable compounds of Formula V and methods of making the same are disclosed in U.S. Patent Publication Ser. No. 2005/0119168, page 3, [0024], through page 215, paragraph [0833], incorporated herein by reference.

Non-limiting examples of suitable compounds of Formula VI and methods of making the same are disclosed in U.S. Patent Publication Ser. No. 2005/0085425 at page 3, paragraph 0023 through page 139, incorporated herein by reference.

Non-limiting examples of suitable compounds of Formula VII, VIII, and IX as well as methods of making the same are disclosed in International Patent Publication WO2005/051980 and in U.S. Patent Publication 2005/0164921 at page 3, paragraph through page 113, paragraph [0271], incorporated herein by reference.

Non-limiting examples of suitable compounds of Formula X and methods of making the same are disclosed in International Patent Publication WO2005/085275 and in U.S. Patent Publication 2005/0267043 at page 4, paragraph [0026] through page 519, paragraph [0444], incorporated herein by reference.

Non-limiting examples of suitable compounds of Formula XI and methods of making the same are disclosed in International Patent Publication WO2005/087721 and in U.S. Patent Publication 2005/0288233 at page 3, paragraph [0026] through page 280, paragraph [0508], incorporated herein by reference.

Non-limiting examples of suitable compounds of Formula XII and methods of making the same are disclosed in International Patent Publication WO2005/087725 and in U.S. Patent Publication 2005/0245458 at page 4, paragraph [0026] through page 194, paragraph [0374], incorporated herein by reference.

Non-limiting examples of suitable compounds of Formula XIII and methods of making the same are disclosed in International Patent Publication WO2005/085242 and in U.S. Patent Publication 2005/0222047 at page 3, paragraph [0026] through page 209, paragraph [0460], incorporated herein by reference.

Non-limiting examples of suitable compounds of Formula XIV and methods of making the same are disclosed in International Patent Publication WO2005/087731 at page 8, line 20 through page 683, line 6, incorporated herein by reference. In particular, the preparation of such compounds including the following structure referred to in International Patent Publication WO2005/087731 as Compound 484

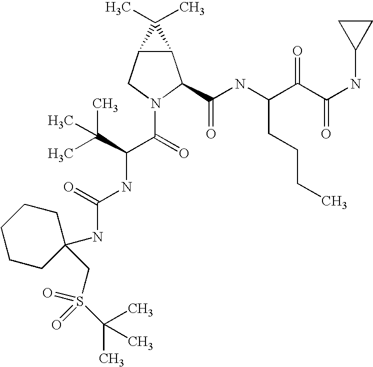

can be found on page 299, Example 792 to page 355, Example 833, incorporated herein by reference.

Non-limiting examples of suitable compounds of Formula XV and methods of making the same are disclosed in International Patent Publication WO2005/058821 and in U.S. Patent Publication 2005/0153900 at page 4, paragraph [0028] through page 83, paragraph [0279], incorporated herein by reference.

Non-limiting examples of suitable compounds of Formula XVI and methods of making the same are disclosed in International Patent Publication WO2005/087730 and in U.S. Patent Publication 2005/0197301 at page 3, paragraph [0026] through page 156, paragraph [0312], incorporated herein by reference.

Non-limiting examples of suitable compounds of Formula XVII and methods of making the same are disclosed in International Patent Publication WO2005/085197 and in U.S. Patent Publication 2005/0209164 at page 3, paragraph [0026] through page 87, paragraph [0354], incorporated herein by reference.

Non-limiting examples of suitable compounds of Formula XVIII and methods of making the same are disclosed in U.S. Patent Publication 2006/0046956, at page 4, paragraph [0024] through page 50, paragraph [0282], incorporated herein by reference.

Non-limiting examples of suitable compounds of Formula XIX and methods of making the same are disclosed in International Patent Publication WO2005/113581 and in U.S. Patent Publication 2005/0272663 at page 3, paragraph [0026] through page 76, incorporated herein by reference.

Non-limiting examples of suitable compounds of Formula XX and methods of making the same are disclosed in International Patent Publication WO 2000/09558 at page 4, line 17 through page 85, incorporated herein by reference.

Non-limiting examples of suitable compounds of Formula XXI and methods of making the same are disclosed in International Patent Publication WO 2000/09543 at page 4, line 14 through page 124, incorporated herein by reference.

Non-limiting examples of suitable compounds of Formula XXII and methods of making the same are disclosed in International Patent Publication WO 2000/59929 and in U.S. Pat. No. 6,608,027, at col. 65, line 65 through col. 141, line 20, each incorporated herein by reference.

Non-limiting examples of suitable compounds of Formula XXIII and methods of making the same are disclosed in International Patent Publication WO02/18369 at page 4, line 4 through page 311, incorporated herein by reference.

Non-limiting examples of suitable compounds of Formula XXIV and methods of making the same are disclosed in U.S. Patent Publication No. 2002/0032175, 2004/0266731 and U.S. Pat. No. 6,265,380 at col. 3, line 35 through col. 121 and 6,617,309 at col. 3, line 40 through col. 121, each incorporated herein by reference.

Non-limiting examples of suitable compounds of Formula XXV and methods of making the same are disclosed in International Patent Publication WO 1998/22496 at page 3 through page 122, incorporated herein by reference.

Non-limiting examples of suitable compounds of Formula XXVI and methods of making the same are disclosed in International Patent Publication WO 1998/17679 at page 5, line 20 through page 108, line 9, incorporated herein by reference.

### Medicaments, Compositions, and Methods

The present invention also provides a pharmaceutical composition comprising a therapeutically effective amount of the medicament and a pharmaceutically acceptable carrier.

The present invention also provides pharmaceutical kits comprising the medicament, in combined or separate unit dosage forms, said forms being suitable for administration of (a) and (b) in effective amounts, and instructions for administering (a) and (b) to treat or ameliorate one or more symptoms associated with HCV infection.

The present invention also provides methods for treating or ameliorating one or more symptoms of HCV, or disorders associated with HCV in a subject in need thereof, comprising administering to the subject an effective amount of the aforementioned medicament.

In one embodiment, the administering is oral, intravenous, intrathecal, parenteral, transdermal, or subcutaneous or a combination of two or more thereof.

In one embodiment, the subject is treatment naïve. In another embodiment, the subject is treatment experienced.

In one embodiment, the subject is co-infected with HIV.

The term “HCV/HIV inhibitor(s)” previously used was meant to encompass one or more inhibitors of HCV and/or HIV.

### HCV Polymerase Inhibitors

HCV polymerase inhibitors suitable for use in the compositions and methods of the present invention include, but are not limited to, compounds disclosed in the following patents and publications, the disclosures of which are incorporated herein by their entirety: US20040023921A1, US20030224469A1, US20060183751A1, US20060183111A1, US20060074035A1, US20030037355A1, U.S. Pat. No. 6,322,966B1, US20010034019A1, US20050153877A1, US20050119318A1, US20050107364A1, US20050048472A1, US20050026923A1, US20040266708A1, US20040229936A1, US20040229840A1, US20040167123A1, US20040158054A1, US20040082075A1, WO2005019191 A2, WO2004041818A1, WO2005095655A1, WO9949031 A1, WO0040759A2, WO9949029A1, U.S. Pat. No. 6,280,940B1, US20050176701A1, EP1256628A2, EP1106702A1, WO2006074346A2, US20020055162A1, WO9800547A1, US6110901A, WO9938985A2, U.S. Pat. No. 5,472,840A, WO2005017133A1, WO2006066079A2, WO2006076650A2, AT407256, WO2003084953A1, WO2006011719A1, WO2004108719A1, WO2004033450A1, WO2004108068A2, DE10225066A1, EP0655505A1, WO2003018832A1, WO0132153A2, WO2004106350A1, US20040014722A1, WO2006050161A2, WO2006002231A1, WO2002069903A2, US20050080053A1, US20040242599A1, US20040229839A1, WO2005021568A2, WO0155702A1, US20040039187A1, WO0053775A2, WO2005019449A2, WO2005053516A2, US20030224977A1, WO2005042530A1, WO2003014377A2, WO2003010141A2, WO2003007945A1, WO0204425A2, WO0183736A2, WO0009558A1, US20030187018A1, US20030186895A1, US20040229818A1, US20040224900A1, WO2006007693A1, WO2005080388A1, WO2005070955A1, WO2005028501A1, WO2004103996A1, WO2004065367A1, WO2004064925A1, WO2004099241A1, WO2005092855A1, WO2006020082A1, WO2005054430A2, WO2005051410A1, WO2005046712A1, WO2005034850A2, WO2004094452A2, WO2004014313A2, WO2003026587A2, WO2002061048A2, CA2370400, JP10165186A, WO0212477A2, WO9702352A1, CN1385540, CN 1526826, CN1757725, WO2005040340A2, WO0157073A2, US20050095582A1, WO0137654A2, WO2003002518A1, WO2002079187A1, WO0208292A2, WO0033635A2, WO9943792A1, U.S. Pat. No. 6,461,845B1, WO2004113365A2, WO2004093798A2, WO2004072243A2, WO2004113555A2, WO2006037102A2, WO2003042385A2, US20030092135A1, WO2004046159A1, WO2003099229A2, WO2004055216A2, WO2003082265A2, WO2005012288A1, US20060111311A1, WO2006076529A1, WO2004028481A2, WO2003093290A2, US20050090463A1, EP0454-461A1, WO0006779A1, WO2005002626A2, WO2006045615A1, WO2006045613A1, WO2005103045A1, WO2005092863A1, WO2005079799A1, WO2004096774A1, WO2004096210A1, WO2004076415A1, WO2004060889A1, WO2004037818A1, WO2004009543A2, WO2003097646A1, WO2003037895A1, WO2003037894A1, WO2003037893A1, WO2003000713A1, WO9936572A1, WO2002093519A2, WO2003077729A2, WO9116902A1, WO0157266A1, WO2006037028A2, WO2003026589A2, WO2004003000A2, WO2006000922A2, WO2004046331A2, WO9203539A1, US20050037018A1, WO0194644A1, WO2006016930A2, WO2005110455A2, WO2005067454A2, WO2005062949A2, WO2005037214A2, WO9967396A1, U.S. Pat. No. 5,576,302A, WO0006529A1, WO2006046030A2, WO2006021449A1, WO2005053670A1, WO2005034941A 1, WO2005023819A1, WO2004110442A1, WO2004087714A1, WO0206246A1, WO9637619A1, WO2006038039A1, WO2006029912A1, WO2006008556A1, WO2003062211A1, WO2006027628A2, WO2006052013A1, WO2005080399A1, WO2005049622A1, WO2005014543A1, US20030050320A1, EP1065213A2, WO0063693A1, KR180274, KR2002070125, KR2003062773, KR2003070240, WO2006033409A1, WO9532200A1, WO2006042327A2, WO2004028471A2, WO2004096993A2, WO2004072090A1, WO2006065335A2, WO2005070957A1, U.S. Pat. No. 6,541,515B2, WO2004007512A2, WO2004003138A2, WO2003020222A2, WO2002057287A2, WO0127309A1, WO9962520A1, WO9962513A1, WO9421797A1, WO2006012078A2, U.S. Pat. No. 7,034,167B2, WO2005123087A2, WO2004009020A2, WO2004000858A2, WO2003105770A2, WO2004011479A1, WO2006037227A1, WO2003028737A1, WO2002051425A1, WO0210396A1, U.S. Pat. No. 5,597,697A, WO2006071619A1, WO0190121 A2, WO2005014806A2, WO2004011624A2, WO2006018725A1, WO2004074270A2, WO2004073599A2, WO2004044228A2, WO2003095441A1, WO2003082848A1, US20050154056A1, WO2004002977A1, WO2004002940A1, WO2005001417A2, WO2004013298A2, WO2005018330A1, WO2005003147A2, WO0204649A2, WO0053784A1, WO0050614A2, WO2002063039A2, WO2006019831A1, WO9933970A1, WO2004065398A2, WO2003062257A1, WO2003051899A1, WO2003051896A1, U.S. Pat. No. 6,906,190B2, WO0116312A2, WO0004141A2, U.S. Pat. No. 6,482,932B1, WO2005000308A2, US20060040927A1, US20060040890A1, U.S. Pat. No. 6,434,489B1, US20060094706A1, WO2006050035A1, WO2006050034A1, WO2005079837A1, WO0158929A1, U.S. Pat. No. 6,472,373B1, U.S. Pat. No. 6,967,075B2, US20040142322A1, DE 102004063132A1, WO2003031645A1, WO0220497A1, WO0177371A1, WO2002100851A2, WO0160315A2, EP1321463A1, WO2002100846A1, WO2003100014A2, WO2003085084A2, WO2003059356A2, WO9929843A1, WO0014252A1, WO0056877A1, WO0189560A1, WO9802530A1, WO2002072776A2, U.S. Pat. No. 6,689,559B2, WO9830238A1, WO9610400A1, U.S. Pat. No. 5,882,852A, JP2002125683A, WO2003015798A1, WO0214362A2, WO0177091A2, EP1619246A1, WO2002095002A2, WO2003006477A1, WO2005037860A2, WO2006050250A2, WO2006039488A2, WO2005077969A2, WO2005043118A2, WO2005042570A1, WO2005042020A2, WO2005035525A2, WO2005007681A2, WO2003035060A1, WO2003006490A1, WO0174768A2, WO0107027A2, WO0024725A1, WO2003087092A2, WO2005028502A1, U.S. Pat. No. 5,837,464A, WO2004089983A2, US20060147997A1, U.S. Pat. No. 5,496,546A, U.S. Pat. No. 6,127,116A, WO2005044986A2, U.S. Pat. No. 6,218,142B1, WO2006065590A2, US20050277613A1, WO2004076621A2. An assay for HCV polymerase inhibitors is described in Harper et al., J Med Chem, 48:1314-1317 (2005).

Notably, HCV polymerase inhibitors suitable for use in the compositions and methods of the present invention exclude HCV-796, identified in the Investigational Drugs database and in the IMS Health database as having the structure shown below:

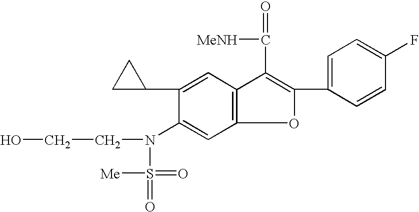

and also identified in the IMS Health database as 5-cyclopropyl-2-(4-fluorophenyl)-6-[(2-hydroxyethyl)(methylsulfonyl)amino]-N-methyl-3-benzofurancarboxamide as well as by the Chemical Abstracts Services (CAS) Number 691852-58-1 which corresponds to the Chemical Abstract index name 3-benzofurancarboxamide, 5-cyclopropyl-2-(4-fluorophenyl)-6-[(2-hydroxyethyl)(methylsulfonyl)amino]-N-methyl, and which is further described in WO 2004041201.

### HCV NS3 Helicase Inhibitors

Examples include compounds, such as those disclosed in, for example, WO 01/07027, herein incorporated by reference.

### Inhibitors of HCV Entry

Examples include antibodies and peptides produced by Innogenetics (e.g., INNO101), XTL (e.g., HCV-Ab^{XTL}68) and Tulane University (e.g., single-chain antibody fragment (scFv) of human monoclonal antibody CM3.B6 which recognizes a conformational epitope within the helicase domain of non-structural 3 protein (NS3) of HCV).

### TLR Agonists

Examples include compounds such as isatoribin and it derivatives (Anadys Pharmaceuticals) or imidaioquinolinamines, such as imiquimod and resiquimod (Dockrell & Kinghom, J. Antimicrob. Chemother., vol 48, pp. 751-755 (2001) and Hemmi et al., Nat. Immunol., vol. 3 pp. 196-200 (2002), guanine ribonucleosides, such as C8-substituted or N7, C-8-disubstituted guanine ribonucleosides (Lee et al., Proc. Natl. Acad. Sci. USA, vol. 100, pp. 6646-6651 (2003) and the compounds that are disclosed in JP-2005-089,334; WO99/32122; WO98/01448 WO05/092893; and WO05/092892, and TLR-7 agonist SM360320 (9-benzyl-8-hydroxy-2-(2-methoxy-ethoxy)adenine) disclosed in Lee et al., Proc Natl Acad Sci USA, 103(6):1828-1833 (2006); all herein incorporated by reference.

In addition to isatoribin, other preferred TLR agonists include 9-benzyl-8-hydroxy-2-(2-methoxyethoxy)adenine (SM360320), ACTILON™ (Coley Pharmaceutical Group, Inc.), and the following compounds by Sumitmo Pharmaceutical Co, Ltd.:

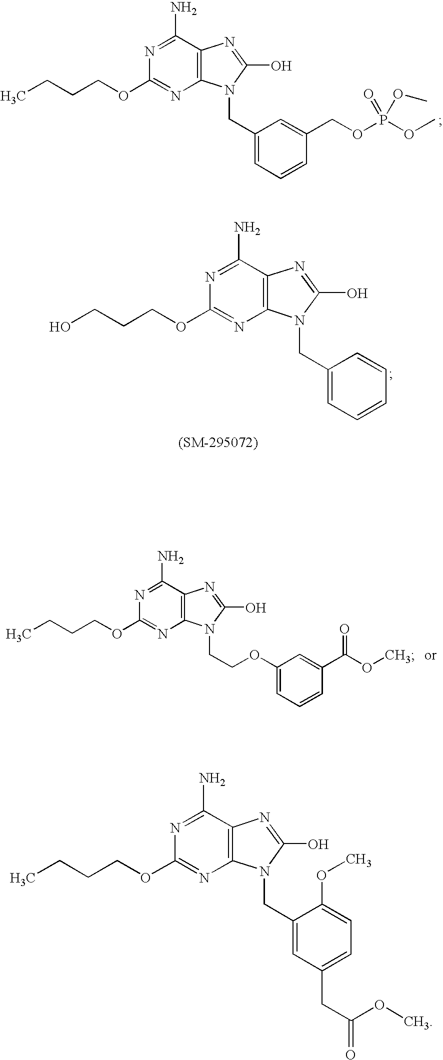

In one embodiment, the TLR-7 agonist is administered in combination with an inosine monophosphate dehydrogenase inhibitor.

Immunomodulatory Agents that Enhance that Antiviral Response

The term “immunomodulatory agent” as used herein refers to an agent which modulates the immune system and thereby has an antiviral effect typically by inducing or eliciting one or more host antiviral mechanisms thus having a negative impact on viral infection or replication by virtue of the immunomodulatory agent's indirect interaction through intermediates produced by or derived from the host. In contrast, the term “antiviral agent” as used herein refers to an agent (e.g., small molecule, oligonucleotide, recombinant protein, or antibody) which has a direct antiviral effect by virtue of its direct interaction with one or more viral proteins or viral nucleic acids (e.g., single stranded or double stranded viral RNAs or DNAs).

Examples of immunomodulatory agents include antibodies that prevent interaction of interleukin-10 (IL-10) with its receptor, such as those disclosed in, for example, US2005/0101770, paragraphs [0086] to [0104], or U.S. Pat. No. 5,863,796, incorporated herein by reference. For example, humanized 12G8, a humanized monoclonal antibody against human IL-10 (plasmids containing the nucleic acids encoding the humanized 12G8 light and heavy chains were deposited with the American Type Culture Collection (ATCC) as deposit numbers PTA-5923 and PTA-5922, respectively).

### AKR Inhibitors

Non-limiting examples of suitable AKR inhibitors include benzodiazepines (e.g., cloxazolam, diazepam, estazolam, flunitrazepam, nitrazepam, medazepam), cyclooxygenase (COX) 2 inhibitors (e.g., celecoxib), non-steroidal anti-inflammatory drugs (NSAIDS), testosterone, and diflunisal.

The AKR inhibitor(s) can be administered to a subject in an amount ranging from about 50 to about 3200 mg per day. Non-limiting examples of suitable dosages can range from about 100 to about 1500 mg per day, preferably about 200 to about 1000 mg/day, and more preferably about 200, about 300, about 400 or about 800 mg per dose, given in a single dose or 2-4 doses per day.

In one embodiment, the medicament further comprises at least one AKR inhibitor. Preferably, at least one AKR inhibitor diflunisal. Preferably, diflunisal is administered at a dosage range of about 1000 mg to about 1500 mg per day.

Preferably, the medicament further comprises at least one AKR inhibitor, preferably diflunisal (at a preferred dosage range of about 1000 mg to about 1500 mg per day) wherein at least one HCV protease inhibitor is:

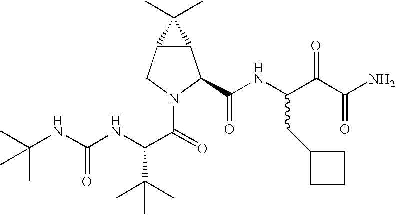

Formula Ia or a pharmaceutically acceptable salt, solvate or ester thereof.

### Pgp Inhibitors

In one embodiment, at least one Pgp inhibitor is selected from the group of Pgp inhibitors referred to in the following documents (which are incorporated by reference herein): US20030139352A1, US20060040908A1, US20020147197A1, US20050171202A1, US20040219609A1, US20040214848A1, US20040110244A1, WO9325705A1, WO0160387A1, WO0059931A1, WO2004019886A2, US20040030248A1, WO0205818A2, WO2002074048A2, WO0123565A1, WO0123540A2, WO0066173A2, WO2006041902A2, WO9600085A1, WO9746254A2, WO2005020962A1, WO0241884A2, U.S. Pat. No. 6,277,655B1, WO2006026592A2, WO2002071061A2, US20040197334A1, WO2006034219A2, WO0174790A2, U.S. Pat. No. 6,376,514B1, WO9962537A1, U.S. Pat. No. 6,521,635B1, WO0125400A2, WO0221135A2, WO0046347A1.

Non-limiting examples of suitable Pgp inhibitors include WK-X-34, ketoconazole (Nizoral™, commercially available from Janssen Pharmaceutica) and ritonavir (Norvir® commercially available from Abbott). Preferably, the Pgp inhibitor is ketoconazole. An assay for Pgp inhibitors is described by Jekerle et al., Int J Cancer, 119(2):414-422 (2006).

In one preferred embodiment, at least one Pgp inhibitor is ritonavir. Preferably, ritonavir is administered at a dosage administered at a dosage of about 400 mg per day.

Compounds that Inhibit HIV

A preferred embodiment for the compounds that inhibit HIV are CCR5 antagonists, such as those described in U.S. Pat. Nos. 6,387,930; 6,602,885; 6,720,325; U.S. Pat. Nos. 6,387,930 and 6,391,865, PCT Publications WO 2000/66558, WO 2000/66559, WO 02/079194, WO 03/69252, WO 03/020716, WO 04/056770, European patent publication EP1421075, and US patent publications US 2004/0092745 and US 2004/0092551 and in U.S. provisional application Ser. No. 60/516,954 filed Nov. 3, 2003, herein incorporated by reference. An especially preferred compound is Vicriviroc.

In an alternative preferred embodiment, the compounds that inhibit HIV are HIV integrase inhibitors, such as those described in, for example, WO 2004/004657, US 2006/0052361 A1; WO01/96283; WO03/016266; WO01/95905; WO03/047564; WO02/30930; WO02/55079; WO03/031413; WO03/335076; WO03/335077; WO04/24078; US 2006/0046985 A1; WO01/00578; US03/0055071; WO02/30426; WO02/55079; WO02/036734; WO03/16275; WO03/35076; WO03/316266; WO03/062204; US 2006/0019906 A1; WO02/070486; WO02/36734; WO02/055079; WO02/070486; WO03/035076; WO03/035077; WO04/046115; U.S. Pat. No. 6,380,249; U.S. Pat. No. 6,306,861; and U.S. Pat. No. 6,262,055, all herein incorporated by reference. An especially preferred HCV integrase inhibitor is Mrk 058 (Merck & Co., Inc.).

Other preferred compounds that inhibit HIV include protease inhibitors (PIs), such as TMC 114 (Tibotec), non-nucleoside reverse transcriptase inhibitors (NNRTI), such as TMC 125 (Tibotec), nucleoside and nucleotide reverse transcriptase inhibitors (NRTI) and fusion inhibitors.

The term “non-nucleoside reverse transcriptase inhibitors” as used herein means non-nucleosides that inhibit the activity of HIV-1 reverse transcriptase.

Typical suitable NNRTIs include nevirapine (BI-RG-587) available under the VIRAMUNE trade name from Boehringer Ingelheim, the manufacturer for Roxane Laboratories, Columbus, Ohio 43216; delaviradine (BHAP, U-90152) available under the RESCRIPTOR trade name from Pharmacia & Upjohn Co., Bridgewater N.J. 08807; efavirenz (DMP-266) a benzoxazin-2-one disclosed in WO94/03440 and available under the SUSTIVA trade name from DuPont Pharmaceutical Co., Wilmington, Del. 19880-0723; PNU-142721, a furopyridine-thio-pyrimide under development by Pharmacia and Upjohn, Bridgewater N.J. 08807; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019 and under clinical development by Agouron Pharmaceuticals, Inc., LaJolla Calif. 92037-1020; MKC-442 (1-(ethoxy-methyl)-5-(1-methylethyl)-6-(phenylmethyl)-(2,4(1H,3H)-pyrimidinedione) discovered by Mitsubishi Chemical Co. and under development by Triangle Pharmaceuticals, Durham, N.C. 27707; and (+)-calanolide A (NSC-675451) and B, coumarin derivatives disclosed in NIH U.S. Pat. No. 5,489,697, licensed to Med Chem Research, which is co-developing (+) calanolide A with Vita-Invest as an orally administrable product.

HIV protease inhibitors refer to compounds that inhibit HIV-1 protease, an enzyme required for the proteolytic cleavage of viral polyprotein precursors (e.g., viral GAG and GAG Pol polyproteins) into the individual functional proteins found in infectious HIV-1. HIV protease inhibitors include compounds having a peptidomimetic structure, high molecular weight (7600 daltons) and substantial peptide character, e.g. CRIXIVAN (available from Merck) as well as nonpeptide protease inhibitors e.g., VIRACEPT (available from Agouron).

Typical suitable PIs include saquinavir (Ro 31-8959) available in hard gel capsules under the INVIRASE trade name and as soft gel capsules under the FORTOVASE trade name from Roche Pharmaceuticals, Nutley, N.J. 07110-1199; ritonavir (ABT-538) available under the NORVIR trade name from Abbott Laboratories, Abbott Park, Ill. 60064; indinavir (MK-639) available under the CRIXIVAN trade name from Merck & Co., Inc., West Point, Pa. 19486-0004; nelfnavir (AG-1343) available under the VIRACEPT trade name from Agouron Pharmaceuticals, Inc., LaJolla Calif. 92037-1020; amprenavir (141 W94), trade name AGENERASE, a non-peptide protease inhibitor under development by Vertex Pharmaceuticals, Inc., Cambridge, Mass. 02139-4211 and available from Glaxo-Wellcome, Research Triangle, N.C. under an expanded access program; lasinavir (BMS-234475) available from Bristol-Myers Squibb, Princeton, N.J. 08543 (originally discovered by Novartis, Basel, Switzerland (CGP-61755); DMP-450, a cyclic urea discovered by Dupont and under development by Triangle Pharmaceuticals; BMS-2322623, an azapeptide under development by Bristol-Myers Squibb, Princeton, N.J. 08543, as a 2nd-generation HIV-1 PI; ABT-378 under development by Abbott, Abbott Park, Ill. 60064; and AG-1549 an orally active imidazole carbamate discovered by Shionogi (Shionogi #S-1153) and under development by Agouron Pharmaceuticals, Inc., LaJolla Calif. 92037-1020.

Other antiviral agents include hydroxyurea, ribavirin, IL-2, IL-12, pentafuside and Yissum Project No. 11607. Hydroyurea (Droxia), a ribonucleoside triphosphate reductase inhibitor, the enzyme involved in the activation of T-cells, was discovered at the NCI and is under development by Bristol-Myers Squibb; in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. RE 33653, 4530787, 4569790, 460-4377, 4748234, 4752585, and 4949314, and is available under the PROLEUKIN (aldesleukin) trade name from Chiron Corp., Emeryville, Calif. 94608-2997 as a lyophilized powder for IV infusion or sc administration upon reconstitution and dilution with water; a dose of about 1 to about 20 million IU/day, sc is preferred; a dose of about 15 million IU/day, sc is more preferred. IL-12 is disclosed in WO96/25171 and is available from Roche Pharmaceuticals, Nutley, N.J. 07110-1199 and American Home Products, Madison, N.J. 07940; a dose of about 0.5 microgram/kg/day to about 10 microgram/kg/day, sc is preferred. Pentafuside (DP-178, T-20) a 36-amino acid synthetic peptide, disclosed in U.S. Pat. No. 5,464,933 licensed from Duke University to Trimeris, which is developing pentafuside in collaboration with Duke University; pentafuside acts by inhibiting fusion of HIV-1 to target membranes. Pentafuside (3-100 mg/day) is given as a continuous sc infusion or injection together with efavirenz and 2 PIs to HIV-1 positive patients refractory to a triple combination therapy; use of 100 mg/day is preferred. Yissum Project No. 11607, a synthetic protein based on the HIV-1 Vif protein, is under preclinical development by Yissum Research Development Co., Jerusalem 91042, Israel. Ribavirin, 1-β-D-ribofuranosyl-1H-1,2,4-triazole-3-carboxamide, is available from ICN Pharmaceuticals, Inc., Costa Mesa, Calif.; its manufacture and formulation are described in U.S. Pat. No. 4,211,771.

Other HIV drugs include, but are not limited to, the following:

### Anti-HIV Drugs

### A. Protease Inhibitors

| Brand Name | Generic Name |
|---|---|
| Agenerase | Amprenavir |
| Aptivus | Tipranavir |
| Crixivan | Indinavir |
| Fortovase | Saquinavir (soft gel cap) |
| Invirase | Saquinavir (hard gel cap) |
| Kaletra | Lopinavir/ritonavir |
| Lexiva | Fosamprenavir |
| Norvir | Ritonavir |
| Reyataz | Atazanavir |
| Viracept | Nelfinavir |

### B. Non Nucleoside Reverse Transcriptase Inhibitors

| Brand Name | Generic Name |
|---|---|
| Rescriptor | Delavirdine |
| Sustiva | Efavirenz |
| Viramune | Nevirapine |

### C. Nucleoside/Nucleotide Reverse Transcriptase Inhibitors

| Brand Name | Generic Name |
|---|---|
| Combivir | Zidovudine + Lamivudine |
| Emtriva | Emtricitabine |
| Epivir | Lamivudine |
| Epzicom | Abacavir + Lamivudine |
| Hivid | Zalcitabine |
| Retrovir | Zidovudine |
| Trizivir | Abacavir + Zidovudine + |
| | Lamivudine |
| Truvada | Tenofovir + Emtricitabine |
| Videx | Didanosine |
| Videx EC | Didanosine: Delayed- |
| | release capsultes |
| Viread | Tenofovir DF |
| Zerit | Stavudine |
| Zerit XR | Stavudine: Delayed- |
| | release |
| Ziagen | Abacavir |

### D. Protease Inhibitors

| Brand Name | Generic Name |
|---|---|
| Fuzeon | Enfuvirtide |

Other antiviral agents that may be used in the present invention include:

| Product | Generic Name |
|---|---|
| Zidovudine | zidovudine |
| Copegus | ribavirin |
| Valaciclovir | valaciclovir |
| Nevirapine | nevirapine |
| Lamivudine | lamivudine |
| Viramidine | taribavirin |
| TMC114 | — |
| TMC125 | etravirine |
| Maraviroc (UK-427,857) | maraviroc |
| LDT600 | telbivudine |
| Telbivudine (LdT) | telbivudine |
| ZYC101a | — |
| Ampligen | — |
| ONO-4128 (873140) | aplaviroc |
| Sustiva/Truvada | efavirenz, tenofovir disoproxil |
| | fumarate & emtricitabine |
| Sustiva/Truvada | efavirenz, tenofovir disoproxil |
| | fumarate & emtricitabine |
| Capravirine/S-1153 | capravirine |
| PRO 2000 | — |
| 873140 (ONO-4128) | aplaviroc |
| Genvir | acyclovir |
| SCH-417690/SCH-D (CCR- | vicriviroc |
| 5 antagonist) | |
| Valopicitabine (NM283) | valopicitabine |
| Valopicitabine (NMC283) | valopicitabine |
| VX-497 | merimepodib |
| TNX-355 | — |
| LDC300 | valtorcitabine |
| Maribavir | maribavir |
| ANA380 | — |
| HepeX-B | libivirumab & exbirivumab |
| Reverset | — |
| Valtorcitabine (LdC) | valtorcitabine |
| ANA380 | — |
| PA-457 | — |
| AI-183 | — |
| BMS-488043 | — |
| Clevudine | clevudine |
| GS 9137 | — |
| Lotreve | loviride |
| TMC278 | rilpivirine |
| c-1605 | — |
| RSV604 | — |
| Intranasal Pleconaril | pleconaril |
| MX-3253 | celgosivir |
| SPD 754 | — |
| Intranasal Pleconaril | pleconaril |
| VX-385 | — |
| Pradefovir | pradefovir |
| TNX-355 | — |
| 640385 | — |
| 695634 | — |
| AG-1859 | — |
| HepeX-B | libivirumab & exbirivumab |
| PRO 542 | — |
| UT-231B | — |
| Intranasal Pleconaril | pleconaril |
| RP-606 (MIV-606) | valomaciclovir |
| BIVN-401 (Virostat) | methylene blue |
| VX-950 | — |
| ANA975 | — |
| HCV-796 | — |
| IL-2 SA | — |
| BILR 355 | — |
| VX-950 | — |
| LY-570310 | — |
| GS 9132 | — |
| R-82150/TMC120 | dapivirine |
| TMC126 | — |
| ANA975 | — |
| R1626 | — |
| CS-8958 | — |
| SCH6 | — |
| TAK-220 | — |
| CCR5-MAb | — |
| ANA975 | — |
| AG1776 | — |
| CI-1029 | — |
| PRO 140 | — |
| XTL-6865 | — |
| PRO 140 | — |
| CCR5-MAb | — |
| UNIL-025 | — |
| HCV-796 | — |
| Hepatitis (InterMune) | — |
| Anti-CMV antibody | — |
| GRN139951 | — |
| GRN140665 | — |
| IL-29 | — |
| BAY 41-4109 | — |
| HCV Program | — |
| HCV-Protease (NS3) | — |
| Inhibitors | |
| TMC254072 | — |
| TMC52390 | — |
| TMC353121 | — |
| NV-05A | — |
| NV-08 | — |
| IL-29 | — |
| R1495 (MV026048) | — |
| HspE7-2nd gen | — |
| R1656 (PSI-6130) | — |
| CS-3955 | — |
| FLUNET | — |
| T-1106 | — |
| PEG-cyanovirin-n | — |
| CS-8958 | — |
| SARS Antibody | — |
| Rabies Antibody | — |
| West Nile Virus Antibody | — |
| VRX773 | — |
| 3B3 (HIV Immunotoxin) | — |
| CMV protease inhibitor | — |
| protease inhibitor | — |
| HSV-1 Protease Inhibitor | — |
| SARS MAb | — |
| HCV-SM | — |
| Research Project | — |
| (VivoQuest) | |
| HuMax-HepC | — |
| ImmStat | — |
| SARS Antisense Research | — |
| Project | |
| MX128533 series | — |
| BCX-4678 | — |
| Peramivir | peramivir |
| PRO 542 | — |
| MPI-49839 | — |
| Iminosugar Platform | — |
| GO 7.1 | — |
| VX-950 | — |
| NV-05A | — |
| NV-08 | — |
| AN 025-1 | — |
| RSV (Trimeris) | — |
| Fusion Inhibitors (Trimeris) | — |
| HCMV Program | — |
| IL-28A | — |
| IL-28B | — |
| Project (Medivir) | — |
| Project (Enanta) | — |
| Project (Gilead) | — |
| Project (Bristol-Myers | — |
| Squibb) | |
| Nucleotide analogues | — |
| Research Project (Chiron) | — |
| Research Project | — |
| (Genelabs) | |
| HCV protease inhibitor | — |
| HCV RNA polymerase | — |
| inhibitor | |
| Sunesis Viral Infection | — |
| Research Project | |
| Anti-Viral Research Project | — |
| ACE2/SARS Research | — |
| Project | |
| Helicase Inhibitor | — |
| HBV Research Project | — |
| Metapneumovirus Antibody | hMPV vaccine |
| Electroporation Program | — |
| (HIV) | |
| Research Project (Dong- | — |
| Wha) | |
| Research Project | — |
| (Hybrigenics) | |
| Therapeutic | — |
| Lassa Fever Antibody | — |
| Anti-Viral MAb Project | — |
| miR-122 antagonist | — |
| MPI-148104 | — |
| MPI-333876 | — |
| RSV Fusion Inhibitor | — |
| Program (Array BioPharma) | |
| Small Molecule Fusion | — |
| Inhibitors (Array BioPharma) | |
| Small Molecule Fusion | — |
| Inhibitors (Neokimia) | |
| Fusion Inhibitors | — |
| (Roche/Trimeris) | |
| Entry Inhibitors (ChemBridge | — |
| Research) | |
| Anti-Viral Research Project | — |
| Next Generation HIV | — |
| Maturation Inhibitor | |
| HIV Fusion Inhibitor | — |
| RSV Fusion Inhibitor | — |
| ANA971 | — |
| SPD 756 | — |
| ANA971 | — |
| SPD 760 | — |
| CGP-61755 | — |
| FB636 | — |
| PG-301029 | — |
| CGP-73547 | atazanavir |
| Bravavir | sorivudine |
| Acyclovir | acyclovir |
| Picovir | pleconaril |
| Picovir | pleconaril |
| Coactinon | emivirine |
| Coviracil (Emtriva) | emtricitabine |
| Lobucavir | ganciclovir |
| Preveon | adefovir dipivoxil |
| RWJ-270201 | peramivir |
| R1461 (HspE7-1st gen) | — |
| Picovir | pleconaril |
| Capravirine | capravirine |
| Coactinon | emivirine |
| R-848 | — |
| KNI-272 | — |
| ABT 606 | — |
| DAPD | amdoxovir |
| L-FMAU | clevudine |
| VP-50406 (HCl-436) | — |
| BAY 40-1007 | — |
| BILN 2061 | ciluprevir |
| MIV-310 | alovudine |
| BMS-234475 | — |
| DPC-684 | — |
| DPC-817 | — |
| DPC-A78277 | — |
| VML 600 | — |
| E3330 | — |
| ISIS 14803 | — |
| LY-466700 | — |
| GS 7340 | — |
| GS 9005 | — |
| Amdoxovir | amdoxovir |
| Clevudine | clevudine |
| MK-944 | — |
| ISIS 13312 | — |
| Ostavir | — |
| PROTOVIR | — |
| T-1249 (R724) | — |
| Levovirin (R1270) | levovirin |
| S-1360 | — |
| KNI-272 | — |
| Levovirin (R1270) | levovirin |
| HBY 097 | — |
| GW420867 | — |
| GW810781 (S-1360) | — |
| Ruprintrivir/AG7088 | ruprintrivir |
| Ostavir | — |
| PROTOVIR | — |
| HepeX-C (AbXTL68) | — |
| AIDS Gene Therapy | — |
| ISIS 14803 | — |
| ISIS 13312 | — |
| Genvir | acyclovir |
| T-1249 | — |
| VP-50406 (HCl-436) | — |
| R803 | — |
| HCV-371 | — |
| HCV-086 | — |
| BAY 38-4766 | — |
| MIV-150 | — |
| Alamifovir (MCC-478) | alamifovir |
| c-2507 | — |
| REV 123 | — |
| R944 (Protease inhibitor) | — |
| R1479 | — |
| R1518 | — |
| R1518 | — |
| DPC-961 | — |
| 204937 (MIV-210) | — |
| 678248 | — |
| MDX-240 | — |
| rhLF | — |
| PRO 367 | — |
| HCV-086 | — |
| HCV-371 | — |
| VP-14637 | — |
| MCC-478 | alamifovir |
| ANA246 | — |
| LdT | telbivudine |
| HCMV Inhibitor | — |
| AIDS-monoclonal antibodies | — |
| NV-08B | — |
| RSC-1838 | — |
| TAK-779 | — |
| LdT | telbivudine |
| HGS-HIV/AIDS 27 | — |
| MLN273 | — |
| ANA246 | — |
| RSC-1838 | — |
| HIV-CA | — |
| Anti-HIV SCA | — |
| MPI-148106 | — |
| RENs & RENt | — |
| RSV backup compound | — |
| NV-08B | — |
| PA-344 | — |
| AN 022-33 | — |
| E913 | — |
| CD4 Attachment Inhibitor | — |
| gp41 Fusion Inhibitor | — |
| Research Project | — |
| Anti-filovirus MAb | — |
| Rhinovirus Polymerase | — |
| Inhibitors | |
| MPYS-174 | — |
| MPYS-188 | — |
| MPYS-763 | — |
| MPYS-900 | — |
| HFV Research Project | — |
| BAY 10-8979 | — |

Isomers (where they exist), including enantiomers, stereoisomers, diastereomers, rotamers, tautomers and racemates are also contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of the present invention. Isomers may also include geometric isomers, e.g., when a double bond is present. Polymorphous forms, whether crystalline or amorphous, also are contemplated as being part of this invention. In particular, the (+) isomers are preferred.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other. For example, both isomers (1) and (2) are contemplated:

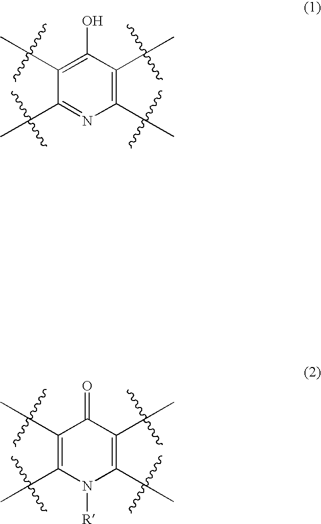

wherein R′ is H or C₁₋₆ unsubstituted alkyl.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (O₂—C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 8-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′are each independently (C₁-C₁₀)alkyl, (C₃-C₇) cycloalkyl, benzyl, or R-carbonyl is a natural α-aminoacyl or natural α-aminoacyl, □C(OH)C(O)OY¹ wherein Y¹ is H, (C₁-C₆)alkyl or benzyl, □C(OY²)Y³ wherein Y² is (C₁-C₄)alkyl and Y³ is (C₁-C₆)alkyl, carboxy (C₁-C₆)alkyl, amino(C₁-C₄)alkyl or mono-N□ or di-N,N—(C₁-C₆)alkylaminoalkyl, □C(Y⁴)Y⁵ wherein Y⁴ is H or methyl and Y⁵ is mono-N□ or di-N,N—(C₁-C₆)alkylamino morpholino, piperidin-1-yl or pyrrolidin-1-yl, and the like.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may also exist as, or optionally converted to, a solvate. Preparation of solvates is generally known. Thus, for example, Caira et al., J Pharm Sci, 93(3):601-611 (2004) describe the preparation of the solvates of the antifungal fluconazole in ethyl acetate as well as from water. Similar preparations of solvates, hemisolvate, hydrates and the like are described by van Tonder et al., AAPS PharmSciTech, 5(1):E12 (2004); and A. L. Bingham et al., Chem. Commun., 603-604 (2001). A typical, non-limiting, process involves dissolving a compound in desired amounts of the desired solvent (organic or water or a mixture thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Therapeutically effective amount” is meant to describe an amount of a medicament, pharmaceutical composition, or combination of the invention effective against HCV to produce the desired therapeutic or ameliorative effect in a suitable human subject. In one aspect of the present invention, the desired therapeutic, ameliorative, inhibitory or preventative effect is to inhibit HCV protease and/or one or more cathepsins in a suitable human subject.

Reference to a compound herein is understood to include reference to salts, esters and solvates thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the various formulae of the present invention may be formed, for example, by reacting a compound of the present invention with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Intl. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention. All acid and base salts, as well as esters and solvates, are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with; for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a Cl_{—}20 alcohol or reactive derivative thereof, or by a 2,3-di (C₆₋₂₄)acyl glycerol.

In such esters, unless otherwise specified, any alkyl moiety present preferably contains from 1 to 18 carbon atoms, particularly from 1 to 6 carbon atoms, more particularly from 1 to 4 carbon atoms. Any cycloalkyl moiety present in such esters preferably contains from 3 to 6 carbon atoms. Any aryl moiety present in such esters preferably comprises a phenyl group.

In another embodiment, this invention provides pharmaceutical compositions comprising the inventive peptides as an active ingredient. The pharmaceutical compositions generally additionally comprise a pharmaceutically acceptable carrier diluent, excipient or carrier (collectively referred to herein as carrier materials). Because of their HCV inhibitory activity, such pharmaceutical compositions possess utility in treating and related disorders.

Another embodiment of the invention discloses the use of the pharmaceutical compositions disclosed above for treatment of diseases such as, for example, HCV, inhibiting cathepsin activity and the like. The method comprises administering a therapeutically effective amount of the inventive pharmaceutical composition to a patient having such a disease or diseases and in need of such a treatment.

In yet another embodiment, the compositions of the invention may be used for the treatment of HCV in humans in combination with at least one other therapeutic agent (e.g., antiviral and/or immunomodulatory agents). Examples of other therapeutic agents include, not are not limited to, Ribavirin (formula L, from Schering-Plough Corporation, Madison, N.J.) and LEVOVIRIN™ (from ICN Pharmaceuticals, Costa Mesa, Calif.), VP 50406™ (from Viropharma, Incorporated, Exton, Pa.), ISIS 14803™ (from ISIS Pharmaceuticals, Carlsbad, Calif.), HEPTAZYME™ (from Ribozyme Pharmaceuticals, Boulder, Colo.), VX 497™ (from Vertex Pharmaceuticals, Cambridge, Mass.), THYMOSIN™ (from SciClone Pharmaceuticals, San Mateo, Calif.), MAXAMINE™ (Maxim Pharmaceuticals, San Diego, Calif.), mycophenolate mofetil (from Hoffman-LaRoche, Nutley, N.J.), interferon (such as, for example, interferon-alpha, PEG-interferon alpha conjugates), antibodies specific to IL-10 (such as those disclosed in US2005/0101770, paragraphs [0086] to [0104] incorporated herein by reference, e.g., humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like. “PEG-interferon alpha conjugates” are interferon alpha molecules covalently attached to a PEG molecule. Illustrative PEG-interferon alpha conjugates include interferon alpha-2a (ROFERON™, from Hoffman La-Roche, Nutley, N.J.) in the form of pegylated interferon alpha-2a (e.g., as sold under the trade name PEGASYS™), interferon alpha-2b (INTRON™, from Schering-Plough Corporation) in the form of pegylated interferon alpha-2b (e.g., as sold under the trade name PEG-INTRON™), interferon alpha-2c (BEROFOR ALPHA™, from Boehringer Ingelheim, Ingelheim, Germany), interferon alpha fusion polypeptides, or consensus interferon as defined by determination of a consensus sequence of naturally occurring interferon alphas (INFERGEN™, from Amgen, Thousand Oaks, Calif.).

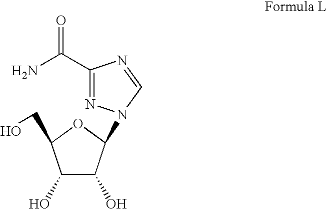

The HCV protease inhibitor and HCV protease inhibitor combination-comprising composition can be administered in combination with interferon alpha, PEG-interferon alpha conjugates or consensus interferon concurrently or consecutively at recommended dosages for the duration of HCV treatment in accordance with the methods of the present invention. The commercially available forms of interferon alpha include interferon alpha 2a and interferon alpha 2b and also pegylated forms of both aforementioned interferon alphas. The recommended dosage of INTRON-A interferon alpha 2b (commercially available from Schering-Plough Corp.) as administered by subcutaneous injection at 3 MIU (12 mcg)/0.5 mL/TIW is for 24 weeks or 48 weeks for first time treatment. The recommended dosage of PEG-INTRON interferon alpha 2b pegylated (commercially available from Schering-Plough Corp.) as administered by subcutaneous injection at 1.5 mcg/kg/week, within a range of 40 to 150 mcg/week, is for at least 24 weeks. The recommended dosage of ROFERON A interferon alpha 2a (commercially available from Hoffmann-La Roche) as administered by subcutaneous or intramuscular injection at 3 MIU (11.1 mcg/mL)/TIW is for at least 48 to 52 weeks, or alternatively 6 MIU/TIW for 12 weeks followed by 3 MIU/TIW for 36 weeks. The recommended dosage of PEGASUS interferon alpha 2a pegylated (commercially available from Hoffmann-La Roche) as administered by subcutaneous injection at 180 mcg/1 mL or 180 mcg/0.5 mL is once a week for at least 24 weeks. The recommended dosage of INFERGEN interferon alphacon-1 (commercially available from Amgen) as administered by subcutaneous injection at 9 mcg/TIW is for 24 weeks for first time treatment and up to 15 mcg/TIW for 24 weeks for non-responsive or relapse treatment. Optionally, Ribavirin, a synthetic nucleoside analogue with activity against a broad spectrum of viruses including HCV, can be included in combination with the interferon and the HCV protease inhibitor. The recommended dosage of ribavirin is in a range from 600 to 1400 mg per day for at least 24 weeks (commercially available as REBETOL ribavirin from Schering-Plough or COPEGUS ribavirin from Hoffmann-La Roche).

The compositions and combinations of the present invention can be useful for treating human subjects of any virus (HCV) genotype. HCV types and subtypes may differ in their antigenicity, level of viremia, severity of disease produced, and response to interferon therapy. (Holland, J. et al., “genotyping by direct sequencing of the product from the Roche Amplicor Test: methodology and application to a South Australian population,” Pathology, 30:192-195, 1998). The nomenclature of Simmonds, P. et al. (“Classification of virus into six major genotypes and a series of subtypes by phylogenetic analysis of the NS-5 region,” J. Gen. Virol., 74:2391-9, 1993) is widely used and classifies isolates into six major genotypes, 1 through 6, with two or more related subtypes, e.g., 1a, 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3. (Lamballerie, X. et al., “Classification of variants in six major types based on analysis of the envelope 1 and nonstructural 5B genome regions and complete polyprotein sequences,” J. Gen. Virol., 78:45-51, 1997). The major genotypes have been defined as having sequence similarities of between 55 and 72% (mean 64.5%), and subtypes within types as having 75%-86% similarity (mean 80%) when sequenced in the NS-5 region. (Simmonds, P. et al., “Identification of genotypes of by sequence comparisons in the core, E1 and NS-5 regions,” J. Gen. Virol., 75:1053-61, 1994).

In another embodiment, the compounds of the invention can be used to treat cellular proliferation diseases. Such cellular proliferation disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or human subjects afflicted or subject to impending affliction with any one of these disorders or states.

The methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma; chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-ydrazone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl) retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, vairubicin, amrubicin, antineoplaston, 3′-deansino-3′-morpholino-13-deoxo-10-hydroxycaminomycin, annamycin, galarubicin, elinafide, MEN10755, 4-demethoxy-3-deamino-3-aziridinyl-4-methylsulphonyl-daunombicin (see WO 00/50032), methotrexate, gemcitabine, and mixture thereof.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino) ethyl]-(20S)camptothecin, BNP1350, BNP11100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-942-[N-[2-(dimethylamino)ethyl]-N-methylamino]ethyl]-5-[4-hydroxy-3,5-dimethoxyphenyl]-5,5a,6,8,8a,9-hexohydrofuro (3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide,N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kif14, inhibitors of Mphosph1 and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-44N242(E),4(E)-tetradecadienoyl]glycylamino]-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-ylacetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin, simvastatin (ZOCOR®), pravastatin (PRAVACHOL®), fluvastatin and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO-96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401 (1999).

Examples of farnesyl protein transferase inhibitors include SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-1-piperidinecarboxamide from Schering-Plough Corporation, Kenilworth, N.J.), tipifarnib (ZARNESTRA® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α (for example INTRON and PEG-INTRON), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). Examples of TAFIa inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

Other combinations encompassed by the present invention include nucleoside and NRTIs, NNRTIs, PIs, other antiviral agents, anti-HIV therapy agents and the like.

The term “nucleoside and nucleotide reverse transcriptase inhibitors” as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA.

Typical suitable NRTIs include zidovudine (AZT) available under the RETROVIR trade name from Glaxo-Wellcome Inc., Research Triangle, N.C. 27709; didanosine (ddI) available under the VIDEX trade name from Bristol-Myers Squibb Co., Princeton, N.J. 08543; zalcitabine (ddC) available under the HIVID trade name from Roche Pharmaceuticals, Nutley, N.J. 07110; stavudine (d4T) available under the ZERIT trademark from Bristol-Myers Squibb Co., Princeton, N.J. 08543; lamivudine (3TC) available under the EPIVIR trade name from Glaxo-Wellcome Research Triangle, N.C. 27709; abacavir (1592U89) disclosed in WO96/30025 and available under the ZIAGEN trademark from Glaxo-Wellcome Research Triangle, N.C. 27709; adefovir dipivoxil [bis(POM)-PMEA] available under the PREVON trade name from Gilead Sciences, Foster City, Calif. 94404; lobucavir (BMS-180194), a nucleoside reverse transcriptase inhibitor disclosed in EP-0358154 and EP-0736533 and under development by Bristol-Myers Squibb, Princeton, N.J. 08543; BCH-10652, a reverse transcriptase inhibitor (in the form of a racemic mixture of BCH-10618 and BCH-10619) under development by Biochem Pharma, Laval, Quebec H7V, 4A7, Canada; emitricitabine [(−)-FTC] licensed from Emory University under Emory Univ. U.S. Pat. No. 5,814,639 and under development by Triangle Pharmaceuticals, Durham, N.C. 27707; beta-L-FD4 (also called beta-L-D4C and named beta-L-2′,3′-dicleoxy-5-fluoro-cytidene) licensed by Yale University to Vion Pharmaceuticals, New Haven Conn. 06511; DAPD, the purine nucleoside, (−)-beta-D-2,6,-diamino-purine dioxolane disclosed in EP 0656778 and licensed by Emory University and the University of Georgia to Triangle Pharmaceuticals, Durham, N.C. 27707; and Iodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc., West Conshohocken, Pa. 19428.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the α_{ν}β₃ integrin, to compounds which selectively antagonize, inhibit or counteract binding of a physiological ligand to the α_{ν}β₅ integrin, to compounds which antagonize, inhibit or counteract binding of a physiological ligand to both the α_{ν}β₃ integrin and the α_{ν}β₅ integrin, and to compounds which antagonize, inhibit or counteract the activity of the particular integrin(s) expressed on capillary endothelial cells. The term also refers to antagonists of the α_{ν}β₆, α_{ν}β₈, α_{ν}β₁, α₂β₁, α₅β₁, α₆β₁ and α₆β₄ integrins. The term also refers to antagonists of any combination of α_{ν}β₃, α_{ν}β₅, α_{ν}β₆, α_{ν}β₈, α₁β₁, α₂β₁, α₅β₁, α₆β₁ and α₆β₄ integrins.

Some examples of tyrosine kinase inhibitors include N-(trifluoromethylphenyl)-5-methylisoxazol-4-carboxamide, 3-[(2,4-dimethylpyrrol-5-yl)methylindenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malignancies. PPAR-γ and PPAR-8 are the nuclear peroxisome proliferator-activated receptors γand δ. The expression of PPAR-γon endothelial cells and its involvement in angiogenesis has been reported in the literature (see J. Cardiovasc. Pharmacol. 1998; 31:909-913; J. Biol. Chem. 1999; 274:9116-9121; Invest. Opthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU 182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy)phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux and a mixture of two or more thereof.

Another embodiment of the present invention is the use of the present compounds in combination with gene therapy for the treatment of cancer. For an overview of genetic strategies to treating cancer, see Hall et al. (Am J Hum Genet. 61:785-789, 1997) and Kufe et al. (Cancer Medicine, 5th Ed, pp 876-889, B C Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The present compounds can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example proclorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, 5,719,147, 7,049,320, and International Patent Application Publication No. WO 2006/007540, the content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-8 agonist, an inhibitor of inherent multidrug resistance, an anti-emetic agent, an immunologic-enhancing drug, an inhibitor of cell proliferation and survival signaling, an agent that interferes with a cell cycle checkpoint, and an apoptosis inducing agent.

Methods for the treatment, prevention or amelioration of one or more symptoms of HCV, treating disorders associated with HCV, modulating activity of HCV, or inhibiting cathepsin activity or associated disorders in a human subject, comprising the step of administering to a human subject in need of such treatment an effective amount of the above compositions or therapeutic combinations, also are provided.

Examples of such cathepsin-associated disorders include proliferative diseases, such as cancer, autoimmune diseases, viral diseases, fungal diseases, neurological/neurodegenerative disorders, arthritis, inflammation, anti-proliferative (e.g., ocular retinopathy), neuronal, alopecia and cardiovascular disease. Many of these diseases and disorders are listed in U.S. Pat. No. 6,413,974, the disclosure of which is incorporated herein.

Other examples of diseases that can be treated include an inflammatory disease, such as organ transplant rejection, graft v. host disease, arthritis, rheumatoid arthritis, inflammatory bowel disease, atopic dermatitis, psoriasis, asthma, allergies, multiple sclerosis, fixed drug eruptions, cutaneous delayed-type hypersensitivity responses, tuberculoid leprosy, type I diabetes, and viral meningitis.

Other examples of diseases that can be treated include Hepatitis B virus and related diseases, Hepatitis A virus and related diseases, HIV and related diseases (e.g., AIDS), and the like.

Another example of a disease that can be treated is a cardiovascular disease.

Other examples of diseases that can be treated include a central nervous system disease, such as depression, cognitive function disease, neurodegenerative disease such as Parkinson's disease, senile dementia such as Alzheimer's disease, and psychosis of organic origin.

Other examples of diseases that can be treated include diseases characterized by bone loss, such as osteoporosis; gingival diseases, such as gingivitis and periodontitis; and diseases characterized by excessive cartilage or matrix degradation, such as osteoarthritis and rheumatoid arthritis.

In one embodiment, the present invention encompasses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treatment naïve” with respect to a human subject refers to one that has never been treated with ribavirin or any interferon including, but not limited to an interferon-alpha. In contrast, the term “treatment experienced” with respect to a human subject refers to one that has been treated with ribavirin or any interferon including, but not limited to an interferon-alpha.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention is a method of treating cancer comprising administering a therapeutically effective amount of at least one compound of the present invention in combination with radiation therapy and at least one compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interferes with a cell cycle checkpoint, and an apoptosis inducing agent.

Yet another embodiment of the invention is a method of treating cancer comprising administering a therapeutically effective amount of at least one compound of the present invention in combination with paclitaxel or trastuzumab.

The present invention also includes a pharmaceutical composition useful for treating or preventing the various disease states mentioned herein cellular proliferation diseases (such as cancer, hyperplasia, cardiac hypertrophy, autoimmune diseases, fungal disorders, arthritis, graft rejection, inflammatory bowel disease, immune disorders, inflammation, and cellular proliferation induced after medical procedures) that comprises a therapeutically effective amount of at least one compound of the present invention and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interferes with a cell cycle checkpoint, and an apoptosis inducing agent.

When the disease being treated by the cathepsin inhibitor compounds of the present invention is inflammatory disease, an embodiment of the present invention comprises administering: (a) a therapeutically effective amount of at least one compound of the present cathepsin inhibitors (e.g., a compound according to Formula I-XXVI) or a pharmaceutically acceptable salt, solvate or ester thereof concurrently or sequentially with (b) at least one medicament selected from the group consisting of: disease modifying antirheumatic drugs; nonsteroidal anti-inflammatory drugs; COX-2 selective inhibitors; COX-1 inhibitors; immunosuppressives (non-limiting examples include methotrexate, cyclosporin, FK506); steroids; PDE IV inhibitors, anti-TNF-α compounds, TNF-alpha-convertase inhibitors, cytokine inhibitors, MMP inhibitors, glucocorticoids, chemokine inhibitors, CB2-selective inhibitors, p38 inhibitors, biological response modifiers; anti-inflammatory agents and therapeutics.

Another embodiment of the present invention is directed to a method of inhibiting or blocking T-cell mediated chemotaxis in a patient in need of such treatment the method comprising administering to the patient a therapeutically effective amount of at least one compound of the present cathepsin inhibitors (e.g., a compound according to Formula I-XXVI) or a pharmaceutically acceptable salt, solvate or ester thereof.

Another embodiment of this invention is directed to a method of treating inflammatory bowel disease in a patient in need of such treatment comprising administering to the patient a therapeutically effective amount of at least one compound according to the present cathepsin inhibitors or a pharmaceutically acceptable salt, solvate or ester thereof.

Another embodiment of this invention is directed to a method of treating or preventing graft rejection in a patient in need of such treatment comprising administering to the patient a therapeutically effective amount of at least one compound according to the present cathepsin inhibitors, or a pharmaceutically acceptable salt, solvate or ester thereof.

Another embodiment of this invention is directed to a method comprising administering to the patient a therapeutically effective amount of: (a) at least one compound according to the present cathepsin inhibitors, or a pharmaceutically acceptable salt, solvate or ester thereof concurrently or sequentially with (b) at least one compound selected from the group consisting of: cyclosporine A, FK-506, FTY720, beta-Interferon, rapamycin, mycophenolate, prednisolone, azathioprine, cyclophosphamide and an antilymphocyte globulin.

Another embodiment of this invention is directed to a method of treating multiple sclerosis in a patient in need of such treatment the method comprising administering to the patient a therapeutically effective amount of: (a) at least one aldo-keto reductase inhibitor and at least one cathepsin inhibitor compound according to the present invention, or a pharmaceutically acceptable salt, solvate or ester thereof concurrently or sequentially with (b) at least one compound selected from the group consisting of: beta-interferon, glatiramer acetate, glucocorticoids, methotrexate, azothioprine, mitoxantrone, VLA-4 inhibitors and/or CB2-selective inhibitors.

Another embodiment of this invention is directed to a method of treating multiple sclerosis in a patient in need of such treatment the method comprising administering to the patient a therapeutically effective amount of the present combination concurrently or sequentially with at least one compound selected from the group consisting of: methotrexate, cyclosporin, leflunimide, sulfasalazine, β-methasone, β-interferon, glatiramer acetate, prednisone, etonercept, and infliximab.

Another embodiment of this invention is directed to a method of treating rheumatoid arthritis in a patient in need of such treatment the method comprising administering to the patient a therapeutically effective amount of the present combination concurrently or sequentially with at least one compound selected from the group consisting of: COX-2 inhibitors, COX inhibitors, immunosuppressives, steroids, PDE IV inhibitors, anti-TNF-α compounds, MMP inhibitors, glucocorticoids, chemokine inhibitors, CB2-selective inhibitors, caspase (ICE) inhibitors and other classes of compounds indicated for the treatment of rheumatoid arthritis.

Another embodiment of this invention is directed to a method of treating psoriasis in a patient in need of such treatment the method comprising administering to the patient a therapeutically effective amount of the present combination concurrently or sequentially with at least one compound selected from the group consisting of: immunosuppressives, steroids, and anti-TNF-α compounds.

Another embodiment of this invention is directed to a method of treating a disease selected from the group consisting of: inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy, type I diabetes, viral meningitis and tumors in a patient in need of such treatment, such method comprising administering to the patient an effective amount of the present combination or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy and cancer in a patient in need of such treatment, such method comprising administering to the patient an effective amount of the present combination or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses and tuberculoid leprosy, type I diabetes, viral meningitis and cancer in a patient in need of such treatment, such method comprising administering to the patient an effective amount of the present combination or a pharmaceutically acceptable salt, solvate or ester thereof concurrently or sequentially with at least one medicament selected from the group consisting of: disease modifying antirheumatic drugs; nonsteroidal anti-inflammatory drugs; COX-2 selective inhibitors; COX-1 inhibitors; immunosuppressives; steroids; PDE IV inhibitors, anti-TNF-α compounds, MMP inhibitors, glucocorticoids, chemokine inhibitors, CB2-selective inhibitors, biological response modifiers; anti-inflammatory agents and therapeutics.

When the present invention involves a method of treating a cardiovascular disease, in addition to administering the amount of the present combination or a pharmaceutically acceptable salt, solvate or ester thereof, the method further comprises administering to the human subject in need one or more pharmacological or therapeutic agents or drugs such as cholesterol biosynthesis inhibitors and/or lipid-lowering agents discussed below.

Non-limiting examples of cholesterol biosynthesis inhibitors for use in the compositions, therapeutic combinations and methods of the present invention include competitive inhibitors of HMG CoA reductase, the rate-limiting step in cholesterol biosynthesis, squalene synthase inhibitors, squalene epoxidase inhibitors and a mixture of two or more thereof. Non-limiting examples of suitable HMG CoA reductase inhibitors include statins such as lovastatin (for example MEVACOR® which is available from Merck & Co.), pravastatin (for example PRAVACHOL® which is available from Bristol Meyers Squibb), fluvastatin, simvastatin (for example ZOCOR® which is available from Merck & Co.), atorvastatin, cerivastatin, rosuvastatin, rivastatin (sodium 7-(4-fluorophenyl)-2,6-diisopropyl-5-methoxymethylpyridin-3-yl)-3,5-dihydroxy-6-heptanoate, CI-981 and pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Preferred HMG CoA reductase inhibitors include lovastatin, pravastatin and simvastatin.

In another embodiment, the method of treatment comprises administering an amount of the present combination or a pharmaceutically acceptable salt, solvate or ester thereof in combination with one or more cardiovascular agents and one or more cholesterol biosynthesis inhibitors.

In another alternative embodiment, the method treatment of the present invention can further comprise administering nicotinic acid (niacin) and/or derivatives thereof, optionally with the cardiovascular agent(s) and sterol absorption inhibitor(s) discussed above.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include niceritrol, nicofuranose and acipimox (5-methylpyrazine-2-carboxylic acid 4-oxide). Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

In another alternative embodiment, the method of treatment of the present invention can further comprise administering one or more AcylCoA:Cholesterol O-acyltransferase (“ACAT”) Inhibitors, which can reduce LDL and VLDL levels, coadministered with or in combination with the cardiovascular agent(s) and sterol absorption inhibitor(s) discussed above. ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See Chong and Bachenheimer, “Current, New and Future Treatments in Dyslipidaemia and Atherosclerosis,” Drugs, 60(1):55-93 (2000), which is incorporated by reference herein.

In another alternative embodiment, the method of treatment of the present invention can further comprise administering probucol or derivatives thereof (such as AGI-1067 and other derivatives disclosed in U.S. Pat. Nos. 6,121,319 and 6,147,250), which can reduce LDL levels, coadministered with or in combination with the cardiovascular agent(s) and sterol absorption inhibitor(s) discussed above.

In another alternative embodiment, the method of treatment of the present invention can further comprise administering fish oil, which contains Omega 3 fatty acids (3-PUFA), which can reduce VLDL and triglyceride levels, coadministered with or in combination with the cardiovascular agent(s) and sterol absorption inhibitor(s) discussed above. Generally, a total daily dosage of fish oil or Omega 3 fatty acids can range from about 1 to about 30 grams per day in single or 2-4 divided doses.

In another alternative embodiment, the method of treatment of the present invention can further comprise administering natural water soluble fibers, such as psyllium, guar, oat and pectin, which can reduce cholesterol levels, coadministered with or in combination with the cardiovascular agent(s) and sterol absorption inhibitor(s) discussed above. Generally, a total daily dosage of natural water soluble fibers can range from about 0.1 to about 10 grams per day in single or 2-4 divided doses.

In another alternative embodiment, the method of treatment of the present invention can further comprise administering plant sterols, plant stanols and/or fatty acid esters of plant stanols, such as sitostanol ester used in BENECOL® margarine, which can reduce cholesterol levels, coadministered with or in combination with the cardiovascular agent(s) and sterol absorption inhibitor(s) discussed above. Generally, a total daily dosage of plant sterols, plant stanols and/or fatty acid esters of plant stanols can range from about 0.5 to about 20 grams per day in single or 2-4 divided doses.

In another alternative embodiment, the method of treatment of the present invention can further comprise administering antioxidants, such as probucol, tocopherol, ascorbic acid, β-carotene and selenium, or vitamins such as vitamin B₆ or vitamin B₁₂, coadministered with or in combination with the at least one aldo-keto reductase inhibitor and at least one cathepsin inhibitor compound according to the present invention. Generally, a total daily dosage of antioxidants or vitamins can range from about 0.05 to about 10 grams per day in single or 2-4 divided doses.

In another alternative embodiment, the method of treatment of the present invention can further comprise administering one or more bile acid sequestrants (insoluble anion exchange resins), coadministered with or in combination with the at least one aldo-keto reductase inhibitor and at least one cathepsin inhibitor compound according to the present invention.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors which bind LDL from plasma to further reduce cholesterol levels in the blood.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WELCHOL® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and a mixture of two or more thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

Also useful with the present invention are methods of treatment that can further comprise administering at least one (one or more) activators for peroxisome proliferator-activated receptors (PPAR). These activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARβ and as NUC1, and each of these names refers to the same receptor.

PPARα regulates the metabolism of lipids. PPARα is activated by fibrates and a number of medium and long-chain fatty acids, and it is involved in stimulating 3-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include the fibrates discussed above.

Other examples of PPARα activators useful with the practice of the present invention include suitable fluorophenyl compounds as disclosed in U.S. Pat. No. 6,028,109 which is incorporated herein by reference; certain substituted phenylpropionic compounds as disclosed in WO 00/75103 which is incorporated herein by reference; and PPARα activator compounds as disclosed in WO 98/43081 which is incorporated herein by reference.

Non-limiting examples of PPARγ activator include suitable derivatives of glitazones or thiazolidinediones, such as, troglitazone (such as REZULIN® troglitazone (-5-[[4-[3,4-dihydro-6-hydroxy-2,5,7,8-tetramethyl-2H-1-benzopyran-2-yl)methoxy]phenyl]methyl]-2,4-thiazolidinedione) commercially available from Parke-Davis); rosiglitazone (such as AVANDIA® rosiglitazone maleate (-5-[[4-[2-(methyl-2-pyridinylamino)ethoxy]phenyl]methyl]-2,4-thiazolidinedione, (Z)-2-butenedioate) (1:1) commercially available from SmithKline Beecham) and pioglitazone (such as ACTOS™ pioglitazone hydrochloride (5-[[4-[2-(5-ethyl-2-pyridinyl)ethoxy]phenyl]methyl]-2,4-]thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARy activator compounds disclosed in U.S. Pat. No. 5,994,554 which is incorporated herein by reference.

Other useful classes of PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 & WO 01/12187 which are incorporated herein by reference; and substituted 4-hydroxy-phenylalconic acid compounds as disclosed in WO 97/31907 which is incorporated herein by reference.

PPARδ compounds are useful for, among other things, lowering triglyceride levels or raising HDL levels. Non-limiting examples of PPARδ activators include suitable thiazole and oxazole derivates, such as C.A.S. Registry No. 317318-32-4, as disclosed in WO 01/00603 which is incorporated herein by reference); certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-difluorophenyl]-1heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; o-anisamide compounds as disclosed in WO 01/21578 which is incorporated herein by reference; and PPAR activator compounds as disclosed in WO 01/40192 which is incorporated herein by reference.

Also useful with the present invention are methods of treatment which further comprise administering hormone replacement agents and compositions. Useful hormone agents and compositions for hormone replacement therapy of the present invention include androgens, estrogens, progestins, their pharmaceutically acceptable salts and derivatives. Combinations of these agents and compositions are also useful.

The cathepsin inhibitors of the present invention are useful in the treatment of central nervous system diseases such as depression, cognitive function diseases and neurodegenerative diseases such as Parkinson's disease, senile dementia as in Alzheimer's disease, and psychoses of organic origin. In particular, the cathepsin inhibitors of the present invention can improve motor-impairment due to neurodegenerative diseases such as Parkinson's disease.

The other agents known to be useful in the treatment of Parkinson's disease which can be administered in combination with the cathepsin inhibitors of the present invention include: L-DOPA; dopaminergic agonists such as quinpirole, ropinirole, pramipexole, pergolide and bromocriptine; MAO-B inhibitors such as deprenyl and selegiline; DOPA decarboxylase inhibitors such as carbidopa and benserazide; and COMT inhibitors such as tolcapone and entacapone.

A preferred dosage for the administration of a composition of the present invention is about 0.001 to 500 mg/kg of body weight/day of a composition of the present invention or a pharmaceutically acceptable salt or ester thereof. An especially preferred dosage is about 0.01 to 25 mg/kg of body weight/day of a composition of the present invention or a pharmaceutically acceptable salt or ester thereof.

The phrases “effective amount” and “therapeutically effective amount” mean that amount of a compound/composition of the present invention, and other pharmacological or therapeutic agents described herein, that will elicit a biological or medical response of a tissue, a system, or a human subject that is being sought by the administrator (such as a researcher or doctor) which includes alleviation of the symptoms of the condition or disease being treated and the prevention, slowing or halting of progression of one or more of the presently claimed diseases. The formulations or compositions, combinations and treatments of the present invention can be administered by any suitable means which produce contact of these compounds with the site of action in the body of, for example, a mammal or human.

For administration of pharmaceutically acceptable salts of the compounds, the weights indicated above refer to the weight of the acid equivalent or the base equivalent of the therapeutic compound derived from the salt.

As described above, this invention includes combinations comprising an amount of at least one CYP3A4 inhibitor and an amount of at least one HCV protease inhibitor, and an amount of one or more additional therapeutic agents listed above (administered together or sequentially) wherein the amounts of the inhibitors result in the desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of the present invention and an additional therapeutic agent may be present in fixed amounts (dosage amounts) in a single dosage unit (e.g., a capsule, a tablet and the like).

If formulated as a fixed dose, such combination products employ the compounds of this invention within the dosage range described herein and the other pharmaceutically active agent or treatment within its dosage range. Compounds of the present invention may also be administered sequentially with known therapeutic agents when a combination formulation is inappropriate. The invention is not limited in the sequence of administration; compounds/compositions of the present invention may be administered either prior to or after administration of the known therapeutic agent. Such techniques are within the skills of persons skilled in the art as well as attending physicians.

The pharmacological properties of the compositions of this invention may be confirmed by a number of pharmacological assays for measuring HCV viral activity or cathepsin activity, such as are well know to those skilled in the art.

While it is possible for the active ingredient to be administered alone, it is preferable to present it as a pharmaceutical composition. The compositions of the present invention comprise at least one active ingredient, as defined above, together with one or more acceptable carriers, adjuvants or vehicles thereof and optionally other therapeutic agents. Each carrier, adjuvant or vehicle must be acceptable in the sense of being compatible with the other ingredients of the composition and not injurious to the mammal in need of treatment.

Accordingly, this invention also relates to pharmaceutical compositions comprising at least one compound utilized in the presently claimed methods, or a pharmaceutically acceptable salt or ester thereof and at least one pharmaceutically acceptable carrier, adjuvant or vehicle.

In yet another embodiment, the present invention discloses methods for preparing pharmaceutical compositions comprising the inventive compounds as an active ingredient. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition. Surfactants may be present in the pharmaceutical formulations of the present invention in an amount of about 0.1 to about 10% by weight or about 1 to about 5% by weight. Acidifying agents may be present in the pharmaceutical formulations of the present invention in a total amount of about 0.1 to about 10% by weight or about 1 to 5% by weight.

Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. HCV inhibitory activity or cathepsin inhibitory activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose.

Preferably the compound is administered orally, intravenously, intrathecally or subcutaneously, parenterally, transdermally or any combination of such methods.

Preferably, the pharmaceutical preparation is in a unit dosage form. In such form, the preparation is subdivided into suitably sized unit doses containing appropriate quantities of the active components, e.g., an effective amount to achieve the desired purpose.

Some useful terms are described below:

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gel—refers to the active ingredients dispersed or solubilized in a hydrophillic semi-solid matrix.

Powder for constitution refers to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition cant range from about 10 to about 90% by weight of the total composition, preferably from about 25 to about 75%, more preferably from about 30 to about 60% by weight, even more preferably from about 12 to about 60%.

Disintegrant—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescents. The amount of disintegrant in the composition can range from about 2 to about 15% by weight of the composition, more preferably from about 4 to about 10% by weight.

Binder—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glident—material that prevents caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

For preparing pharmaceutical compositions from the combinations described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a human subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Preferably the composition is administered orally, intravenously or subcutaneously.

Preferably, the pharmaceutical preparation is in a unit dosage form. In such form, the preparation is subdivided into suitably sized unit doses containing appropriate quantities of the active component, e.g., an effective amount to achieve the desired purpose.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compositions of the present invention and/or the pharmaceutically acceptable salts or esters thereof will be regulated according to the judgment of the attending clinician considering such factors as age, condition and size of the patient as well as severity of the symptoms being treated. A typical recommended daily dosage regimen for oral administration can range from about 1 mg/day to about 3000 mg/day, inclusive of each amount therebetween, preferably about 50 mg/day to about 800 mg/day, in two to four divided doses. In another embodiment, the daily dosage can range from about 50 to about 600 mg/day. In another embodiment, the daily dosage can range from about 50 to about 400 mg/day. In another embodiment, the daily dosage can range from about 50 to about 200 mg/day. Preferably, the dosage is 400 mg/TID.

The compositions of the present invention preferably are administered in an amount effective to reduce the concentration of HCV RNA per milliliter of plasma to a level of less than about 29 IU/mL. The term “concentration of less than 29 International Units of HCV RNA per milliliter of plasma (29 IU/mL)” in the context of the present invention means that there are fewer than 29 IU/ml of HCV RNA, which translates into fewer than 100 copies of HCV-RNA per ml of plasma of the patient as measured by quantitative, multi-cycle reverse transcriptase PCR methodology. HCV-RNA is preferably measured in the present invention by research-based RT-PCR methodology well known to the skilled clinician. This methodology is referred to herein as HCV-RNA/qPCR. The lower limit of detection of HCV-RNA is 29 IU/ml or 100 copies/ml. Serum HCV-RNA/qPCR testing and HCV genotype testing will be performed by a central laboratory. See also J. G. McHutchinson et al. (N. Engl. J. Med., 1998, 339:1485-1492), and G. L. Davis et al. (N. Engl. J. Med. 339:1493-1499).

### Assay for HCV Protease Inhibitory Activity:

### Spectrophotometric Assay:

Spectrophotometric assay for the HCV serine protease can be performed on the inventive compounds by following the procedure described by R. Zhang et al., Analytical Biochemistry, 270 (1999) 268-275, the disclosure of which is incorporated herein by reference. The assay based on the proteolysis of chromogenic ester substrates is suitable for the continuous monitoring of HCV NS3 protease activity. The substrates are derived from the P side of the NS5A-NS5B junction sequence (Ac-DTEDWX(Nva), where X=A or P) whose C-terminal carboxyl groups are esterified with one of four different chromophoric alcohols (3- or 4-nitrophenol, 7-hydroxy-4-methyl-coumarin, or 4-phenylazophenol). Illustrated below are the synthesis, characterization and application of these novel spectrophotometric ester substrates to high throughput screening and detailed kinetic evaluation of HCV NS3 protease inhibitors.

### Materials and Methods:

Materials: Chemical reagents for assay related buffers are obtained from Sigma Chemical Company (St. Louis, Mo.). Reagents for peptide synthesis were from Aldrich Chemicals, Novabiochem (San Diego, Calif.), Applied Biosystems (Foster City, Calif.) and Perseptive Biosystems (Framingham, Mass.). Peptides are synthesized manually or on an automated ABI model 431A synthesizer (from Applied Biosystems). UV/VIS Spectrometer model LAMBDA 12 was from Perkin Elmer (Norwalk, Conn.) and 96-well UV plates were obtained from Corning (Corning, N.Y.). The prewarming block can be from USA Scientific (Ocala, Fla.) and the 96-well plate vortexer is from Labline Instruments (Melrose Park, Ill.). A Spectramax Plus microtiter plate reader with monochrometer is obtained from Molecular Devices (Sunnyvale, Calif.).

### Enzyme Preparation:

Recombinant heterodimeric HCV NS3/NS4A protease (strain 1a) is prepared by using the procedures published previously (D. L. Sali et al., Biochemistry, 37 (1998) 3392-3401). Protein concentrations are determined by the Biorad dye method using recombinant HCV protease standards previously quantified by amino acid analysis. Prior to assay initiation, the enzyme storage buffer (50 mM sodium phosphate pH 8.0, 300 mM NaCl, 10% glycerol, 0.05% lauryl maltoside and 10 mM DTT) is exchanged for the assay buffer (25 mM MOPS pH 6.5, 300 mM NaCl, 10% glycerol, 0.05% lauryl maltoside, 5 μM EDTA and 5 μM DTT) utilizing a Biorad Bio-Spin P-6 prepacked column.

### Substrate Synthesis and Purification:

The synthesis of the substrates is done as reported by R. Zhang et al., (ibid.) and is initiated by anchoring Fmoc-Nva-OH to 2-chlorotrityl chloride resin using a standard protocol (K. Barlos et al., Int. J. Pept. Protein Res., 37 (1991), 513-520). The peptides are subsequently assembled, using Fmoc chemistry, either manually or on an automatic ABI model 431 peptide synthesizer. The N-acetylated and fully protected peptide fragments are cleaved from the resin either by 10% acetic acid (HOAc) and 10% trifluoroethanol (TFE) in dichloromethane (DCM) for 30 min, or by 2% trifluoroacetic acid (TFA) in DCM for 10 min. The combined filtrate and DCM wash is evaporated azeotropically (or repeatedly extracted by aqueous Na₂CO₃ solution) to remove the acid used in cleavage. The DCM phase is dried over Na₂SO₄ and evaporated.

The ester substrates are assembled using standard acid-alcohol coupling procedures (K. Holmber et al., Acta Chem. Scand., B33 (1979) 410-412). Peptide fragments are dissolved in anhydrous pyridine (30-60 mg/ml) to which 10 molar equivalents of chromophore and a catalytic amount (0.1 eq.) of para-toluenesulfonic acid (pTSA) were added. Dicyclohexylcarbodiimide (DCC, 3 eq.) is added to initiate the coupling reactions. Product formation is monitored by HPLC and can be found to be complete following 12-72 hour reaction at room temperature. Pyridine solvent is evaporated under vacuum and further removed by azeotropic evaporation with toluene. The peptide ester is deprotected with 95% TFA in DCM for two hours and extracted three times with anhydrous ethyl ether to remove excess chromophore. The deprotected substrate is purified by reversed phase HPLC on a C3 or C8 column with a 30% to 60% acetonitrile gradient (using six column volumes). The overall yield following HPLC purification can be approximately 20-30%. The molecular mass can be confirmed by electrospray ionization mass spectroscopy. The substrates are stored in dry powder form under desiccation.

### Spectra of Substrates and Products:

Spectra of substrates and the corresponding chromophore products are obtained in the pH 6.5 assay buffer. Extinction coefficients are determined at the optimal off-peak wavelength in 1-cm cuvettes (340 nm for 3-Np and HMC, 370 nm for PAP and 400 nm for 4-Np) using multiple dilutions. The optimal off-peak wavelength is defined as that wavelength yielding the maximum fractional difference in absorbance between substrate and product (product OD−substrate OD)/substrate OD).

### Protease Assay:

HCV protease assays are performed at 30° C. using a 200 μl reaction mix in a 96-well microtiter plate. Assay buffer conditions (25 mM MOPS pH 6.5, 300 mM NaCl, 10% glycerol, 0.05% lauryl maltoside, 5 μM EDTA and 5 μM DTT) are optimized for the NS3/NS4A heterodimer (D. L. Sali et al., ibid.)). Typically, 150 μl mixtures of buffer, substrate and inhibitor are placed in wells (final concentration of DMSO 4° A) v/v) and allowed to preincubate at 30° C. for approximately 3 minutes. Fifty μls of prewarmed protease (12 nM, 30° C.) in assay buffer, is then used to initiate the reaction (final volume 200′ p1). The plates are monitored over the length of the assay (60 minutes) for change in absorbance at the appropriate wavelength (340 nm for 3-Np and HMC, 370 nm for PAP, and 400 nm for 4-Np) using a Spectromax Plus microtiter plate reader equipped with a monochrometer (acceptable results can be obtained with plate readers that utilize cutoff filters). Proteolytic cleavage of the ester linkage between the Nva and the chromophore is monitored at the appropriate wavelength against a no enzyme blank as a control for non-enzymatic hydrolysis. The evaluation of substrate kinetic parameters is performed over a 30-fold substrate concentration range (˜6-200 μM). Initial velocities are determined using linear regression and kinetic constants are obtained by fitting the data to the Michaelis-Menten equation using non-linear regression analysis (Mac Curve Fit 1.1, K. Raner). Turnover numbers (k_{cat}) are calculated assuming the enzyme is fully active.

### Evaluation of Inhibitors and Inactivators:

The inhibition constants (Kᵢ) for the competitive inhibitors Ac-D-(D-Gla)-L-1-(Cha)-C—OH (27), Ac-DTEDVVA(Nva)-OH and Ac-DTEDVVP(Nva)-OH are determined experimentally at fixed concentrations of enzyme and substrate by plotting vₒ/vᵢ vs. inhibitor concentration ([I]ₒ) according to the rearranged Michaelis-Menten equation for competitive inhibition kinetics: vₒ/vᵢ=1+[I]ₒ/(Kᵢ(1+[S]ₒ/Kₘ)), where vₒ is the uninhibited initial velocity, vᵢ is the initial velocity in the presence of inhibitor at any given inhibitor concentration ([I]ₒ) and [S]ₒ is the substrate concentration used. The resulting data are fitted using linear regression and the resulting slope, 1/(Kᵢ(1+[S]ₒ/Kₘ), is used to calculate the Kᵢ value.

Incubation Studies of Compound Formula Ia or Compound Formula XXVII with AKR Inhibitor or CYP3A4 Inhibitor

Pooled human liver microsomes (1 nmol P450/mL) and cytosol (1.6 mg/mL) were incubated with 1 and 20 μM Formula XXVII for 30 and 60 min respectively, in the presence of an NADPH-generating system (1 mM NADP, 5 mM glucose-6-phosphate and 1.5 units/mL glucose-6-phosphate dehydrogenase) and 3 mM magnesium chloride in 0.5 mL of 100 mM potassium phosphate buffer, pH 7.4. Prior to the addition of drug, the incubation mixture was preincubated for 2 min at 37° C. Reactions were initiated by addition of drug, allowed to proceed for up to 30 or 60 min at 37° C., and then terminated by the addition of 0.5 mL of ice-cold acetonitrile with 1% acetic acid. The incubation mixture was vortexed and centrifuged (˜10,000 g) at 4° C. for 15 min and supernatants were analyzed by LC-MS. Human liver microsomes and cytosol without NADPH served as negative controls. Parallel incubations with the compound of Formula Ia were used as positive controls.

Inhibition of Formula XXVII metabolism was evaluated using selective chemical inhibitors of aldo-keto reductase (100 μM flufenamic acid, 50 μM mefenamic acid, 200 μM diflunisal and 100 μM phenolphthalein) and CYP3A4 (2 μM ritonavir and 2 μM ketoconazole). Human liver cytosol (1.6 mg protein/mL) was pre-incubated separately with various inhibitors for 15 min at room temperature followed by the addition of buffer, cofactor and substrate (20 μM). All incubations were performed as described previously for human liver cytosols. Incubation volumes were 0.5 mL and the final concentration of the organic solvents in the incubation system was less than or equal to 1% (v/v). Reactions were initiated by addition of substrate, allowed to proceed for 60 min at 37° C., and then terminated by the addition of 0.5 mL of ice-cold acetonitrile with 1% acetic acid. The incubation mixture was vortexed and centrifuged (˜10,000 g) at 4° C. for 10 min; supernatants were analyzed by LC-MS. Parallel incubations with the compound of Formula Ia were used as positive controls.

Following incubation of Formula XXVII with human liver (HL) cytosol, an ‘M+2’ metabolite (m/z=680) was formed apparently by a metabolic pathway similar to that for the formation of the ‘M+2’ metabolite (m/z=522) from the compound of Formula Ia following similar incubations. Formation of the ‘M+2’ metabolite from Formula XXVII was inhibited 2- to 4-fold following incubations of Formula)(XVII in human liver cytosol in presence of AKR inhibitors such as flufenamic acid, mefenamic acid, diflunisal, and phenolphthalein (see Table 1). Formation of the ‘M+2’ metabolite from the compound of Formula Ia following similar incubations was inhibited 3- to 8-fold.

Metabolic inhibition of liver cytosolic enzymes (including AKRs) can be used clinically for improving the pharmacokinetics (PK) and/or pharmacodynamics (PD)/therapeutic outcome of Formula XXVII and the compound of Formula Ia resulting in either lower doses and/or decrease in dosing frequency.

Additional metabolic inhibition can be obtained clinically by concomitant inhibition of alternate metabolic pathways for the metabolism of Formula XXVII and/or the compound of Formula Ia, i.e., concomitant inhibition of the cytochrome P450 pathway by inhibitors of these enzymes (e.g., ritonavir or ketoconazole as inhibitors of CYP3A4 and other enzymes/transporters) would provide PK and/or PD benefit over and above that achievable by inhibition separately. Concomitant use of inhibitors of parallel metabolic/transport pathways other than the AKR pathway would allow inhibition of these pathways that would otherwise be involved from the diversion of metabolism resulting from inhibition of the AKR pathway for example.

TABLE 1
Incubation of compound Formula Ia or compound Formula
XXVII with AKR inhibitor or CYP3A4 inhibitor.

| | | | | % M + 2/ | |
|---|---|---|---|---|---|
| | | 1^{ST} PARENT | 1^{ST} M + 2 | PARENT | FOLD |
| COMPOUND | MATRICES | PEAK AREA | PEAK AREA | INITIAL | INHIBITION |
| Formula | HL Cytosol w/o | 7.41E+07 | 1.93E+06 | 2.60 | |
| Ia | NADPH | | | | |
| Formula | HL Cytosol w/o | 3.03E+08 | 0.00E+00 | 0.00 | |
| XXVII | NADPH | | | | |
| Formula | HL Cytosol w/ | 3.95E+07 | 6.78E+07 | 91.49 | |
| Ia | NADPH Vehicle | | | | |
| | Control | | | | |
| Formula | HL Cytosol w/ | 3.03E+08 | 2.09E+07 | 6.90 | |
| XXVII | NADPH Vehicle | | | | |
| | Control | | | | |
| Formula | HL Cytosol w/ | 3.81E+07 | 6.63E+07 | 89.40 | 1 |
| Ia | NADPH + 2 uM | | | | |
| | Ritonavir | | | | |
| Formula | HL Cytosol w/ | 2.98E+08 | 1.98E+07 | 6.53 | 1 |
| XXVII | NADPH + 2 uM | | | | |
| | Ritonavir | | | | |
| Formula | HL Cytosol w/ | 6.33E+07 | 1.75E+07 | 23.57 | 4 |
| Ia | NADPH + 100 uM | | | | |
| | Flufenamic acid | | | | |
| Formula | HL Cytosol w/ | 3.08E+08 | 7.82E+06 | 2.58 | 3 |
| XXVII | NADPH + 100 uM | | | | |
| | Flufenamic acid | | | | |
| Formula | HL Cytosol w/ | 6.19E+07 | 2.13E+07 | 28.68 | 3 |
| Ia | NADPH + 50 uM | | | | |
| | Mefenamic acid | | | | |
| Formula | HL Cytosol w/ | 2.92E+08 | 9.48E+06 | 3.13 | 2 |
| XXVII | NADPH + 50 uM | | | | |
| | Mefenamic acid | | | | |
| Formula | HL Cytosol w/ | 6.10E+07 | 9.02E+06 | 12.18 | 8 |
| Ia | NADPH + 200 uM | | | | |
| | Diflunisal | | | | |
| Formula | HL Cytosol w/ | 2.88E+08 | 6.55E+06 | 2.16 | 3 |
| XXVII | NADPH + 200 uM | | | | |
| | Diflunisal | | | | |
| Formula | HL Cytosol w/ | 6.23E+07 | 1.18E+07 | 15.90 | 6 |
| Ia | NADPH + 100 uM | | | | |
| | Phenolphthalein | | | | |
| Formula | HL Cytosol w/ | 2.86E+08 | 4.89E+06 | 1.61 | 4 |
| XXVII | NADPH + 100 uM | | | | |
| | Phenolphthalein | | | | |

### Clinical Study to Evaluate the Effect of Ketoconazole (CYP3A4 and Pgp Inhibitor) or Ibuprofen (AKR Inhibitor) on the Pharmacokinetics and Metabolism of Formula Ia

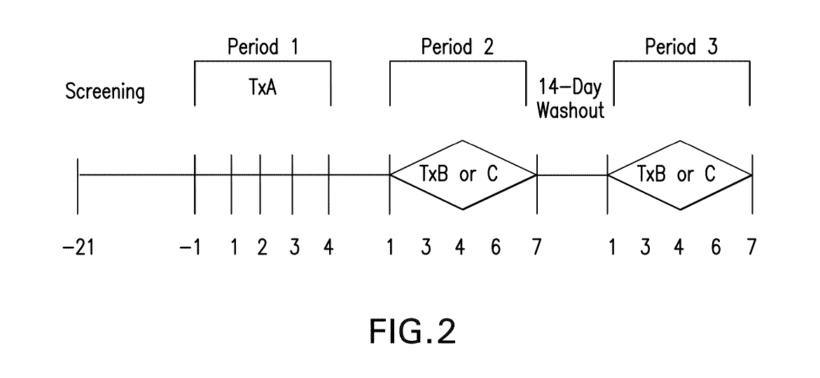

The study was conducted in an open-label, randomized, 3-period, 2-sequence crossover manner (FIG. 2). During Period 1, all 12 human subjects were administered a single 400 mg dose of Formula Ia. During Periods 2 and 3, human subjects received multiple doses of interacting drug, either ketoconazole (400 mg BID) or ibuprofen (600 mg TID) in a randomized sequence. The interacting drug was administered beginning on Day 1 (3 days prior to Formula Ia administration) and continued through Day 6. A single dose of Formula Ia was administered on Day 4 (2 hours after administration of the AM dose of interacting drug). Plasma samples for pharmacokinetic and metabolite analyses of Formula Ia was collected at predose (0 hour), 0.5, 1, 1.5, 2, 2.5, 3, 4, 5, 6, 7, 8, 9, 10, 12, 24, 36, 48, and 72 hour postdose for each period. (The 48 and 72 hour postdose samples for Period 1 was collected in an outpatient setting). In Periods 2 and 3, additional blood samples were collected immediately prior to dosing of the Formula Ia on Day 4 and two hours post ketoconazole/ibuprofen administration on Day 5 for determination of ketoconazole or ibuprofen concentration.
Treatment A: Formula Ia (4×100 mg capsules); single dose, P.O following an overnight fast, administered on Day 1 or Period 1.
Treatment B: Ketoconazole 400 mg; PO, administered BID from Day 1 to Day 6, Formula Ia (4×100 mg capsules); single dose, PO following an overnight fast, administered on Day 4 (2 hours after the AM ketoconazole dose).
Treatment C: Ibuprofen 600 mg; PO, TID from Day 1 to Day 6 Formula Ia (4×100 mg capsules); single dose, PO following an overnight fast, administered on Day 4 (2 hours after the AM ibuprofen dose).

Human subjects received a single dose of Formula Ia on Day 1 of Period 1. In Period 2 and Period 3, human subjects were treated for 6 days with either ketoconazole or ibuprofen and received a single dose of Formula Ia on Day 4 of each period. There were at least 7 days between administration of Formula Ia in Period 1 and Period 2 and at least 14 days between administration of Formula Ia in Period 2 and 3.

The proportion of human subjects with plasma concentrations above the in vitro IC₅₀ and IC₉₀ for the HCV replicon at each time point was determined. This plasma concentration data was used to estimate the following primary pharmacokinetic variables for the determination of bioavailability comparisons:
AUC(tf)—Area under the plasma concentration-time curve from Time 0 to infinity.
Cmax—Maximum observed plasma concentration.
Tmax—Time to maximum observed plasma concentration.
t½—Terminal phase half-life.

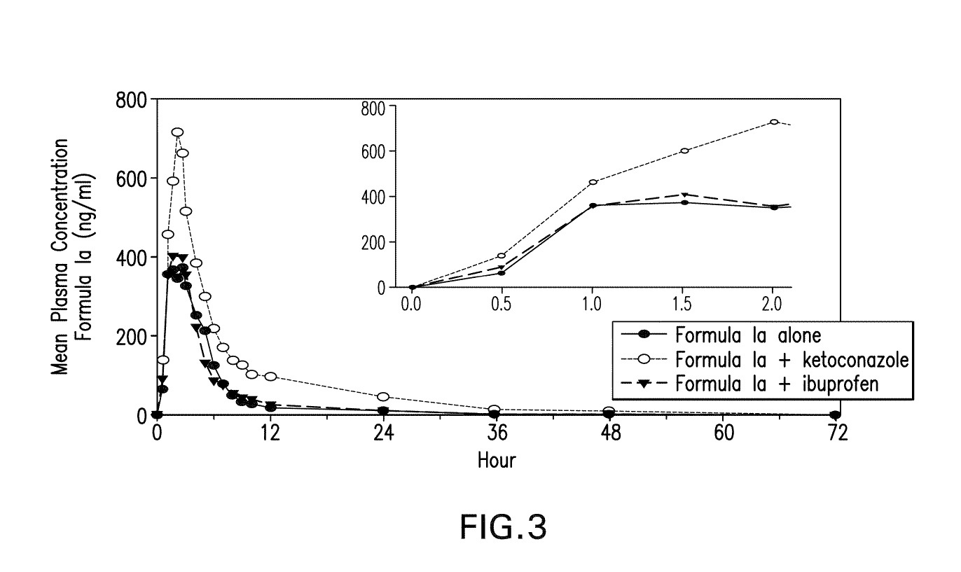

Coadministration of ketoconazole resulted in a prolonged exposure for Formula Ia and a 2-fold increase in the bioavailability of Formula Ia as compared to monotherapy of Formula Ia alone (see FIG. 3). This effect is attributed to the enhancement of both the rate and extent of absorption of Formula Ia (FIG. 3 with inset). The relative bioavailabilities of Formula Ia administered in the presence of the interacting drugs compared to Formula Ia administered alone are shown in Table 2.

TABLE 2
Comparison between Formula Ia treatment alone, Formula Ia
co-administered with ketoconazole or Formula Ia co-administered
with ibuprofen for major PK parameters.
Mean (% CV) PK Parameters

| | | Formula Ia + | Formula Ia + |
|---|---|---|---|
| | Formula Ia | ketoconazole | ibuprofen |
| Cmax | 571 (45) | 830 (48) | 642 (87) |
| AUClast | 2001 (59) | 4565 (36) | 2013 (47) |
| AUCall | 2044 (58) | 4639 (36) | 2055 (45) |
| AUC(l) | 2067 (57) | 4660 (37) | 2090 (44) |
| C8 | 48.0 (38) | 137 (51) | 54.3 (65) |
| t½ | 9.11 (59) | 7.71 (37) | 8.02 (51) |
| MRT (l) | 6.57 (30) | 9.44 (32) | 6.91 (28) |
| t½ eff | 3.3 (26) | 5.96 (34) | 4.16 (35) |
| Tmax (median) | 1.75 | 2.00 | 2.00 |

A comparison between Formula Ia treatment alone and Formula Ia co-administered with ketoconazole or Formula Ia co-administered with ibuprofen for several PK parameters is displayed in Table 3. Co-administering ketoconazole with Formula Ia increased the overall exposure of Formula Ia by more than 2-fold (AUC) and increased the trough concentration (C8) by approximately 3-fold. The increase in Cmax was moderate (average of 40%).

TABLE 3
Comparison between Formula Ia treatment alone and
Formula Ia co-administered with ketoconazole or Formula
Ia co-administered with ibuprofen for several PK parameters.

| | |
|---|---|
| Formula Ia + | Formula Ia + |
| ketoconazole | ibuprofen |

| Parameter | Ratio (%) | 90% CI | Ratio (%) | 90% CI |
|---|---|---|---|---|
| Cmax | 140 | 98-200 | 94 | 65-136 |
| AUCₗₐₛₜ | 238 | 198-287 | 104 | 90-121 |
| AUC (l) | 233 | 195-275 | 104 | 90-120 |
| C8 | 309 | 239-401 | 118 | 82-169 |

It has been well documented in the literature that ketoconazole is a potent inhibitor of CYP3A4 and that it interacts with Pgp (gene product of mdr1 gene). Formula Ia appears to be a substrate for CYP3A4 and Pgp as the increase in bioavailability when combined with ketoconazole probably reflects both an increase in absorption due to inhibition of Pgp-mediated intestinal efflux and a decrease in clearance due to inhibition of CYP3A4-mediated metabolism. In addition, the mean residence time (MRT) and effective half-life of Formula Ia were increased by ketoconazole, an effect most consistent with a decrease of clearance of Formula Ia due to inhibition of CYP3A4/5.

Clinical Study to Assess the Pharmacokinetics, Safety, and Tolerability of Formula Ia Administered in Combination with Ritonavir

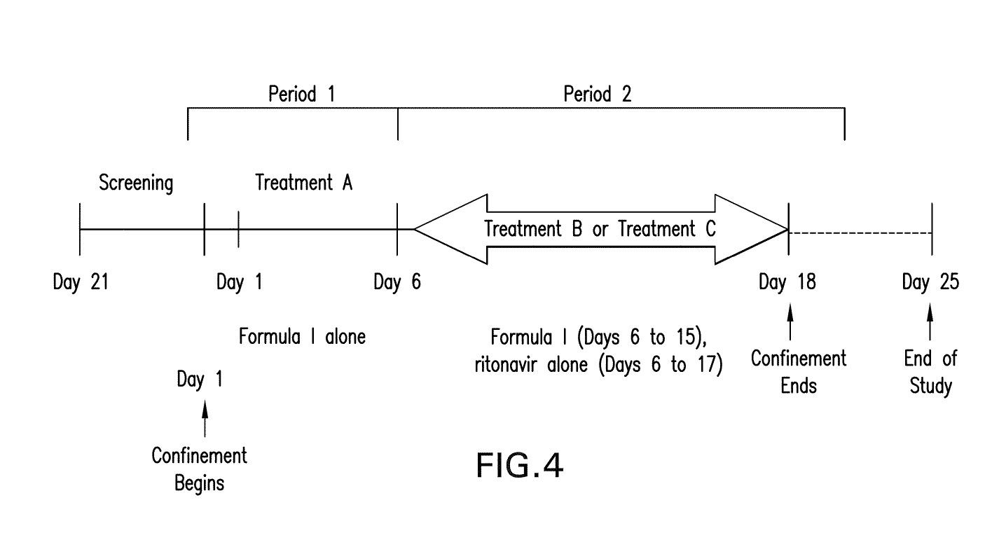

This study was an open-label, randomized, 2-period fixed-sequence, multiple-dose study (FIG. 4). The safety of coadministration of Formula Ia and ritonavir, as well as the quantitation of the ability of ritonavir to enhance Formula Ia PK parameters (specifically trough concentration values) in healthy human subjects was explored. A dose of 400 mg TID of Formula Ia coadministered with ritonavir was selected, as we have substantial safety and PK data available with Formula Ia administered alone at 400 mg TID and 800 TID for comparison. The dose selected of ritonavir was at a level to inhibit CYP3A4 and below the therapeutic dose for HIV.

Although the half life of ritonavir is approximately 3 to 5 hours, the inhibitory effects may last longer. In this study, the effect of ritonavir on Formula Ia was examined as a low dose (100 mg) at two different dosing frequencies (i.e., once in the morning (QAM) and twice a day (BID)), which are commonly administered in HIV therapy. Based upon the findings of these regimens, subsequent regimens may be explored, with modification of the Formula Ia and/or ritonavir component(s).

Human subjects received Formula Ia alone for 5 days in order to achieve steady-state. Human subjects were then randomized to receive one of two treatment regimens in which ritonavir was coadministered with Formula Ia (Formula Ia for 10 days, ritonavir administered for 12 days). Steady-state PK samples for Formula Ia were collected on Day 5 (Formula Ia alone), and on Day 15 (Formula Ia+ritonavir) and the PK parameters (primarily trough concentrations values) compared. Ritonavir was administered alone on Days 16 and 17 to maintain inhibition while the terminal t½ of Formula Ia and Formula Ia metabolites (Formula Ia′; Formula Ic) were assessed.

It has been shown that the exposure to Formula Ia increases when coadministered with food. Food also increases the tolerability to ritonavir. In this study, Formula Ia and ritonavir were administered with food to allow the assessment of safety at maximum exposure. The 400 mg dose for Formula Ia was chosen as there is at least a 4-fold exposure multiple noted in the most sensitive animal species as compared with the mean exposure to Formula Ia noted in humans receiving 400 mg thrice-a-day (TID).

In Period 1, all 16 human subjects received Treatment A and in Period 2 human subjects were randomized to either Treatment B or Treatment C (8 human subjects/treatment).

Period 1 (Days 1 to 5): Treatment A: Formula Ia 400 mg TID, every 8 hours) (Q8° following a meal or snack.

Period 2 (Days 6 to 17): Treatment B: Formula Ia 400 mg TID (Q8°, Days 6 to 15), ritonavir 100 mg QAM (Days 6 to 17), following a meal or snack; Treatment C:

Formula Ia 400 mg BID, every 12 hours (Q12°), (Days 6 to 15), ritonavir 100 mg BID, Q12° (Days 6 to 17), following a meal or snack.

Safety parameters including vital signs, laboratory tests, and ECG were monitored throughout the study. PK samples for Formula Ia, Formula Ic, Formula Ia′, and ritonavir were collected on Days 15, 16, 17, and 18. Serum Inhibin B and semen samples were collected throughout the study. See FIG. 4 for a schematic of this clinical study.

### Test Product, Dose, Mode of Administration

Formula Ia (2×200 mg 3% SLS containing capsules), PO, TID, following a meal or snack.
Formula Ia (2×200 mg 3% SLS containing capsules), PO, BID, following a meal or snack.
Ritonavir (1×100 mg capsules), PO, QAM, following a meal or snack.
Ritonavir (1×100 mg capsules), PO, BID, following a meal or snack.

### Duration of Treatment

Seventeen days; 5 days Formula Ia alone, 10 days Formula Ia in combination with ritonavir and 2 days of ritonavir alone.

### Safety and Tolerability

The overall Safety and tolerability evaluation included all safety data (safety labs, ECGs, AEs and vital signs).

### Pharmacokinetics

The trough levels after multiple-dosing of Formula Ia alone (Day 5) and after multiple-dosing of Formula Ia in combination with ritonavir (Day 15) were compared. The following parameters of Formula Ic (active diastereomer) and Formula Ia were determined: AUC, Cmax, Cmin, and Tmax. The following parameters of Formula Ia′ (metabolite) are reported in Table 4: AUC, Cmax and Tmax. The t½ (based on data through 72 hours postdose), Vd/F, and CL/F will be reported for combination administration only if data permit.

### Safety

Adverse events were tabulated by treatment. ECG parameters were looked at and reviewed as well as the safety laboratory tests and vital signs.

### Pharmacokinetics

Plasma Formula Ia concentrations and pharmacokinetic parameters were listed and summarized using descriptive statistics.

The primary pharmacokinetic parameter is Cmin. The secondary parameters are Cmax and AUC. The log transformed pharmacokinetic parameters including Cmin, AUC, and Cmax were statistically analyzed using ANOVA model extracting effects due to treatment and human subject. The point estimates of the mean difference between Treatment B (Formula Ia 400 mg TID+ritonavir 100 mg QAM) or Treatment C (Formula Ia 400 mg BID+ritonavir 100 mg BID) versus Treatment A (Formula Ia 400 mg TID) were calculated. The corresponding 90% confidence intervals were also provided. There is no intention to compare Treatments B and C to each other.

### Period 1 (Days 1 to 5)

Treatment A: Formula Ia 400 mg TID (Q8°) following a meal or snack. Period 2 (Days 6 to 17).
Treatment B: Formula Ia 400 mg TID (Q8°, Days 6 to 15), ritonavir 100 mg QAM (Days 6 to 17) following a meal or snack.
Treatment C: Formula Ia 400 mg BID (Q12° Days 6 to 15), ritonavir 100 mg BID (Q12°, Days 6 to 17) following a meal or snack.

This study was designed to determine the effect of ritonavir on the trough concentration value of Formula Ia, as well as other pharmacokinetic profile parameters (AUC, Cmax, Tmax, t½ of Formula Ia).

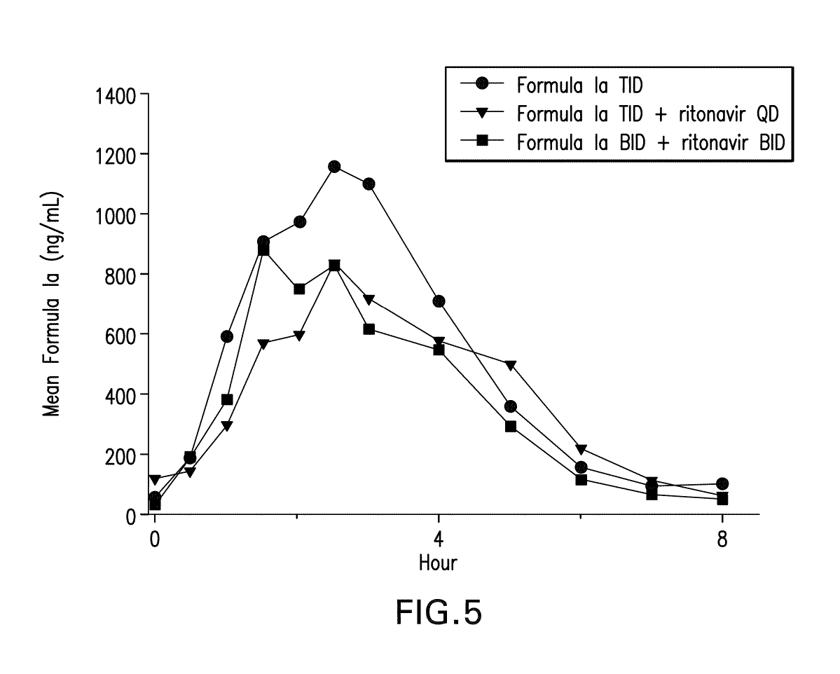

Coadministration of Formula Ia with 100 mg ritonavir QD or BID dosing had no effect on the PK parameters examined compared to monotherapy of Formula Ia alone (see FIG. 5). The relative bioavailabilities of Formula Ia administered in the presence and absence of ritonavir are shown in Table 4.

TABLE 4
Comparison between Formula Ia treatment alone and Formula Ia
co-administered with ritonavir for several PK parameters.

| | Mean PK (% CV) | | |
|---|---|---|---|
| | | Formula Ia TID + | Formula Ia TID + |
| | Formula Ia TID | ritonavir QD | ritonavir BID |
| Cmax | 1358 (11) | 876 (22) | 907 (7) |
| AUC8 | 4116 (9) | 3248 (15) | 3158 (20) |
| Tmax | 2.13 (35) | 3.25 (76) | 0.71 (35) |
| C8 | 104 (31) | 64.3 (45) | 51.8 (10) |
| C12 | — | — | 8.5 (12) |

Clinical Study to Assess the Pharmacokinetics, Safety, and Tolerability of Formula XIVa after Multiple-Dose Administrations with Increasingly Higher Doses, as Well as Administered in Combination with Ritonavir

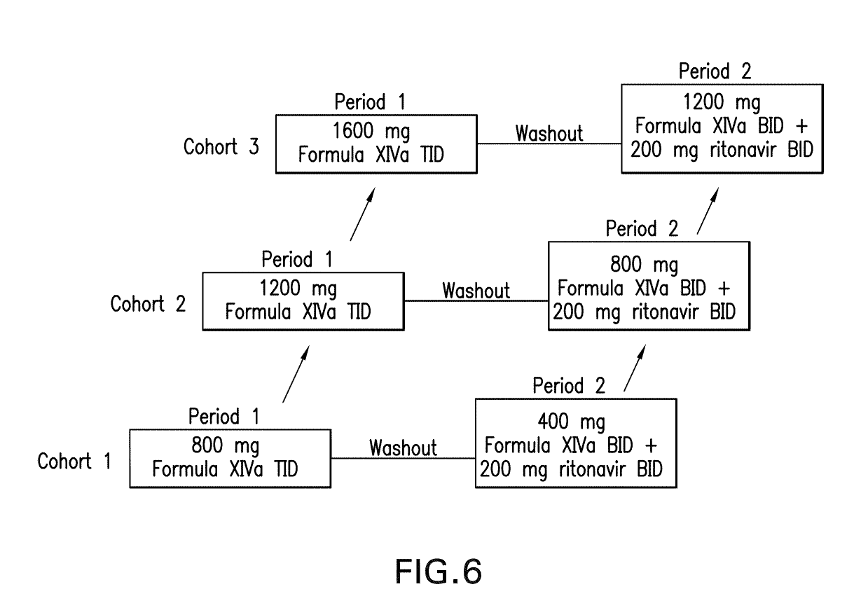

This study will be a randomized, 2-period fixed-sequence, multiple-dose study to assess the pharmacokinetics, safety, and tolerability of Formula XIVa (FIG. 6). In addition, the safety of Formula XIVa administered in combination with ritonavir, as well as the quantitation of enhancement of Formula XIVa PK parameters (specifically trough concentration values) in healthy human subjects will be explored.

### Rising Multiple Dose (RMD) (Period 1)

Subjects will be treated with multiple doses of amorphous Formula XIVa (800 mg, 1200 mg, and 1600 mg TID) or placebo suspension for 11 days (Cohort 1) or 6 days (Cohorts 2 and 3). Within each dose group, 6 subjects will be randomized to receive active drug and 2 subjects will receive placebo. Subjects will be admitted to the study center on Day −2 for baseline assessments. On Day −1, subjects will have serial vital sign and ECG measurements recorded. On Day 1, the subjects will receive a single dose of Formula XIVa or placebo following a high-fat breakfast and will undergo extensive PK sampling (predose, 1, 2, 3, 4, 5, 6, 7, 8, 10, 12, 16, and 24 hours postdose). On Day 2, subjects will begin to receive multiple doses of Formula XIVa (or placebo) TID. Treatment will be administered Q8H: in the morning (at approximately 8 AM) following a high-fat breakfast, in the afternoon (at approximately 4 PM) following a high-fat snack, and at night (at approximately 12 PM) following a high-fat snack. The first dose level will be 800 mg. For Cohort 1, subjects will continue with 800 mg TID of Formula XIVa (or placebo) through Day 10. For Cohorts 2 and 3, subjects will continue with 1200 mg or 1600 mg TID of Formula XIVa, respectively, (or placebo) through Day 5. On Day 11 for Cohort 1 and Day 6 for Cohorts 2 and 3, subjects will receive a single AM dose of Formula XIVa (or placebo) following a high-fat breakfast and will undergo extensive PK sampling once again. On the final study day, safety assessment will again be performed and subjects will be discharged. Samples will be collected for safety assessments throughout the study. Progression to each successive dose level will occur only after safety and tolerability (review of safety laboratory tests, ECGs, vital signs, and adverse event occurrences) of the completed dose (Period 1 of each cohort) have been established and will be agreed upon by the sponsor and the principal investigator.

### Drug-Drug Interaction (DDI) (Period 2)

After an interdose interval of approximately 7 days, subjects will return to be treated with multiple doses of amorphous Formula XIVa (400 mg, 800 mg, and 1200 mg BID) or placebo suspension for 11 days in combination with 200 mg ritonavir BID. Cohort 1 will receive 400 mg Formula XIVa or placebo BID with 200 mg ritonavir BID, Cohort 2 will receive 800 mg of Formula XIVa or placebo BID with 200 mg ritonavir BID, and Cohort 3 will receive 1200 mg of Formula XIVa or placebo with 200 mg ritonavir BID. Within each cohort, 6 subjects will receive active drug and 2 subjects will receive placebo according to the randomization assigned in Period 1. Subjects will be admitted to the study center of Day −2 for baseline assessments to confirm eligibility. On Day −1, subjects will have serial vital sign and ECG measurements recorded. On Day 1, the subjects will receive a single dose of Formula XIVa or placebo and will undergo extensive PK sampling (predose 1, 2, 3, 4, 5, 6, 7, 8, 10, 12, 16, and 24 hours postdose). On Day 2, subjects will begin to receive multiple doses of Formula XIVa (or placebo) BID and 200 mg ritonavir BID. Treatment with both Formula XIVa and ritonavir will be administered Q12H: in the morning (at approximately 8 AM) following a standard high fat breakfast and at night (at approximately 8 PM) following a standard high fat dinner. The first dose level of Formula XIVa in combination with ritonavir will be 400 mg. For all three cohorts, subjects will continue with 400 mg, 800 mg, or 1200 mg BID of Formula XIVa (or placebo) in combination with ritonavir through Day 10. On Day 11 for all three Cohorts, subjects will receive a single AM dose of Formula XIVa (or placebo) and ritonavir BID and will undergo extensive PK sampling once again. Comparison of the pharmacokinetic profile of Formula XIVa pre- and post-treatment with ritonavir will assess whether ritonavir can improve the trough levels of the drug and whether ritonavir in combination with Formula XIVa can reduce the dosing frequency of the drug. On Day 12, safety assessments will again be performed and subjects will be discharged from the study. Samples will be collected for safety assessments throughout the study. Progression to each successive dose level will occur only after safety and tolerability (review of safety laboratory tests, ECGs, vital signs, and adverse event occurrences) of the completed dose (Period 2 of each Cohort) have been established and will agreed upon by the sponsor and the principal investigator.

### Test Product, Dose, Mode of Administration

Each Cohort is comprised of two periods:
Period 1: 800 mg, 1200 mg, or 1600 mg Formula XIVa or placebo TID
Period 2: 400 mg, 800 mg, or 1200 mg Formula XIV1 or placebo BID+200 mg ritonavir BID
   Cohort 1 Period 1 (RMD): Amorphous Formula XIVa, single 800 mg dose (AM) followed by 800 mg TID for 9 days and then a single 800 mg dose (AM) for 1 day administered as an oral suspension.
   Cohort 2 Period 1 (RMD): Amorphops Formula XIVa, single 1200 mg dose (AM) followed by 1200 mg TID for 4 days and then a single 1200 mg dose (AM) for 1 day administered as an oral suspension.
   Cohort 3 Period 1 (RMD): Amorphous Formula XIVa, single 1600 mg dose (AM) followed by 1600 mg TID for 4 days and then a single 1600 mg dose (AM) for 1 day administered as an oral suspension.
   All Cohorts Period 2 (DDI): Amorphous Formula XIVa, as a single 400 mg, 800 mg, or 1200 mg dose (AM), followed by 400 mg, 800 mg, or 1200 mg BID for 9 days, then a single 400 mg, 800 mg, or 1200 mg dose (AM) for 1 day administered as an oral suspension in combination with 200 mg rionavir (2×100 mg capsule) BID on Days 2 to 11.

Notably, all treatments will be administered with a high-fat meal or snack.

### Reference Therapy, Dose, Mode of Administration

Placebo, multiple dose, administered as an oral suspension to match the Formula XIVa treatment. Notably, all treatments will be administered with a high-fat meal or snack.

### Duration of Treatment

All subjects will participate in two treatment periods; the two periods will be separated by a washout period of approximately 7 days.

Period 1 (RMD): Subjects in Cohort 1 will receive treatment (Formula XIVa or matching placebo) for 11 days. Subjects in Cohorts 2 and 3 will receive treatment (Formula XIVa or matching placebo) for 6 days.
Period 2 (DDI): All subjects will be treated with Formula XIVa or matching placebo in combination with rionavir for 11 days.

### Safety and Tolerability

Adverse events, ECGs, vital signs, urinalysis, and laboratory values will be listed for each subject and tabulated by treatment and summarized using descriptive statistics.

### Pharmacokinetics

Single and multiple plasma Formula XIVa concentrations and pharmacokinetic parameters will be listed and summarized using descriptive statistics and graphically displayed by day and dose/regimen. Point estimate along with 90% confidence intervals will be provided for each day and dose/regimen based on log-transformed AUC, Cmax, C8, and C12.

To assess preliminary multiple dose proportionality, log transformed, dose normalized AUC and Cmax at the last day will be analyzed separately for each period using one way ANOVA extracting the effect due to dose. Steady state will be characterized using Days 3, 4, and 5 (or 7, 8, 9, and 10) trough concentrations for each dose/regimen.

To characterize the Formula XIVa pharmacokinetic exposure with and without ritonavir, concentrations of Formula XIVa at 8 and 12 hours after dose will be summarized and graphically displayed by dose/regimen. The number of subjects whose concentration levels are above EC90 (30 ng/ml) at 8 or 12 hours post dose will be tabulated by dose/regimen. In addition, the number of subject whose concentration levels are above the EC90 at their lowest concentration and the fold above EC90 at that time point will be listed.

Ritonavir plasma concentrations will be listed and summarized using descriptive statistics.

Preliminary analysis will include examining the pharmacokinetic parameters for extreme values by reviewing the standardized ranges of deviations from the expected value derived from the model to see if any value exceeds 3. The impact of any outlier on the results of the analyses will be calculated.

A Phase II clinical study of HCV positive patients treated with recombinant human IL-10 showed that treatment was associated with an increase in viral load and a decrease in hepatic fibrosis (see, e.g., Nelson et al., Hepatology, 38(4):859-868 (2003)), suggesting a role of IL-10 in maintenance of chronic HCV infection and its pathogenic sequelae, and further suggesting that anti-IL-10 could be of clinical benefit as an adjunct to the molecules of the present invention for chronic HCV hepatitis.

### Pre-Clinical Study to Assess the Efficacy of Humanized Monoclonal Antibody Against Human IL-10

Humanized 12G8, a humanized monoclonal antibody against human IL-10 previously shown to bind and neutralize the biological activity of recombinant chimpanzee IL-10, was administered to chimpanzees chronically infected with HCV. The primary endpoint for this study was viral load in blood serum measured by reverse transcriptase polymerase chain reaction (RT-PCR).

Chimpanzees (Pan troglodytes; Southwest Foundation for Biomedical Research (SFBR, New Mexico) chronically infected with HCV genotype 1a and persistently mild to moderate elevations in ALT/AST were used for the study. The chimpanzees were group housed in individual cages and offered a nutritionally adequate ration (Heartland Monkey Chow) ad libitum, replaced twice per day, with tap water provided ad libitum. Chimpanzees received supportive care including antibiotics, analgesics and minor surgery as determined to be medically necessary by the study veterinarian.

A solution of humanized 12G8 solution was used for injection at a concentration 24.1 mg/ml. Intravenous injection into the cephalic vein was as a bolus over 5-10 minutes at a dosage of 10 mg/kg. The chimpanzees were monitored for blood pressure, heart rate and respiration during infusion. Administration was once every 14-day period for 2 months, for a total of 5 injections. The first day of dosing was designated as Day 0. The actual volume administered to each animal was calculated from the most recent body weight data.

Blood for serum assays was collected into serum separator tubes then centrifuged to obtain the serum. The serum was then collected, split into 1 ml aliquots, and placed in a −80° C. freezer within 2 hours of the blood sample collection.

Total liver or serum RNA was isolated using RNazol (Leedo, Houston, Tex.). Replicon RNA was quantified by a real time, 5′ exonuclease RT-PCR (Taqman) assay as described in Lanford et al., J Gen Virol, 82(Pt 6):1291-1297 (2001). The primers and probe were derived from the 5′ non-coding region (NCR) and were selected using the Primer Express software designed for this purpose (PE Biosystems). The primers and probe were used at 10 μmol/50 μl reaction. The reactions were performed using the BRILLIANT PLUS SINGLE STEP RT-PCR Kit (Stratagene, La Jolla, Calif.) and included a 30 min 48° C. reverse transcription step, followed by 10 min at 95° C., and then 40 cycles of amplification using the universal Taqman RT-PCR standardized conditions; 15 sec at 95° C. for denaturation and 1 min at 60° C. for annealing and extension. Standards to establish genome equivalents were synthetic RNAs transcribed from a clone of the 5′ NCR of the HCV-1 strain (see, Lanford et al., J Gen Virol, 82(Pt 6):1291-1297 (2001)). Synthetic RNA was prepared using the T7 Megascript Kit and was purified by DNase treatment, RNazol extraction, and ethanol precipitation. RNA was quantified by optical density and 10-fold serial dilutions were prepared from 1 million to 10 copies using tRNA as a carrier. These standards were run in all TAQMAN RT-PCR assays in order to calculate genome equivalents in the experimental samples.

Two chimpanzees completed the study and one chimpanzee was lost from the study due to an intrahepatic bleed as a complication of liver biopsy.

Overall, the study showed that chronic HCV-1a infected chimpanzees treated with humanized 12G8 was safe and well-tolerated. Immunomodulatory effects on liver-infiltrating T-cells were observed in both chimpanzees. A decrease in viral load was observed in one animal that paralleled decreases in a serum marker for liver inflammation (GGT) as well as decreases in tissue expression of several chemokines associated with inflammation. These observations suggest that treatment with anti-IL-10 may be of benefit in treatment of chronic HCV infection.

Reduction of viral load (i.e., the number of HCV genomes per ml of serum) is a well accepted marker of response to anti-viral therapy (see, e.g., Flamm, JAMA, 289(18):2413-2417 (2003)). Viral loads in chimpanzees 4×0174 and 4×0216 at study initiation were in the range of 1e5 to 5e6 genomes per ml, typical of chronically-infected humans and chimpanzees. Measurements of viral load in untreated humans and chimpanzees fluctuate over time, and changes of 0.5 to 1 log are not unusual. Viral load measures in animal 4×0262, were relatively stable prior to and during treatment with humanized 12G8, then trended consistently downward after Week 10. Viral loads of animal 4×0174 showed some fluctuation during the course of treatment. Overall, the downward trend in animal 4×216, with a 1 log drop in viral load at the end of the study, is suggestive of an antiviral effect of humanized 12G8 treatment.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Each document (including granted patents, published patent applications, and nonpatent publications such as journal articles) referred, to in this application is incorporated in its entirety by reference for all purposes.

1-31. (canceled)

32. A medicament comprising, separately or together:
(a) at least one cytochrome P450 isoenzyme 3A4 (CYP3A4) inhibitor; and
(b) at least one hepatitis C virus (HCV) protease inhibitor which is: 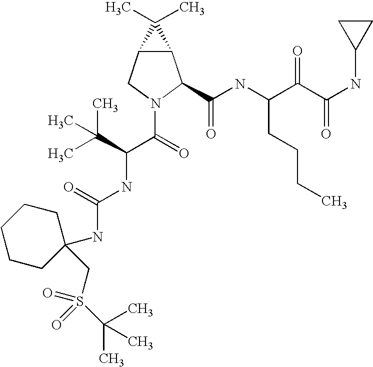
Formula XIVa or a pharmaceutically acceptable salt thereof;
for concurrent or consecutive administration in treating or ameliorating one or more symptoms of HCV in a subject in need thereof.

33. The medicament of claim 32, further comprising at least one other therapeutic agent.

34. The medicament of claim 33, wherein at least one other therapeutic agent is an interferon.

35. The medicament of claim 34, further comprising ribavirin.

36. The medicament of claim 33, wherein at least one other therapeutic agent is ribavirin.

37-49. (canceled)

50. A method for treating or ameliorating one or more symptoms of HCV or disorders associated with HCV in a human subject in need thereof, comprising the step of administering to the human subject an effective amount of the medicament of claim 32.

51. The method of claim 50, wherein the human subject is treatment naïve.

52. The method of claim 50, wherein the human subject is treatment experienced.

53. The method of claim 50, wherein the human subject is co-infected with HIV.

54-78. (canceled)

79. The medicament of claim 34, wherein the interferon is a pegylated interferon.

80. The medicament of claim 34, wherein the interferon is interferon-alpha, PEG-interferon alpha conjugate, interferon alpha fusion polypeptide, consensus interferon, or a mixture of two or more thereof.

81. The medicament of claim 33, wherein at least one other therapeutic agent is interferon, ribavirin, levovirin, VP 50406, ISIS 14803, Heptazyme, VX 497, Thymosin, Maxamine, mycophenolate mofetil, or an interleukin-10 (IL-10) antagonist or an IL-10 receptor antagonist.

82. The medicament of claim 32, wherein at least one CYP3A4 inhibitor is ritonavir, ketoconazole, clarithromycin, BAS 100, itraconazole, nelfinavir, indinavir, erythromycin, troleandomycin, saquinavir, nefazodone, fluconazole, one or more furocoumarin or furocoumarin dimers, fluoxetine, fluvoxamine, clotrimazole, midazolam, naringenin, bergamottin, a compound disclosed selected from the group consisting of 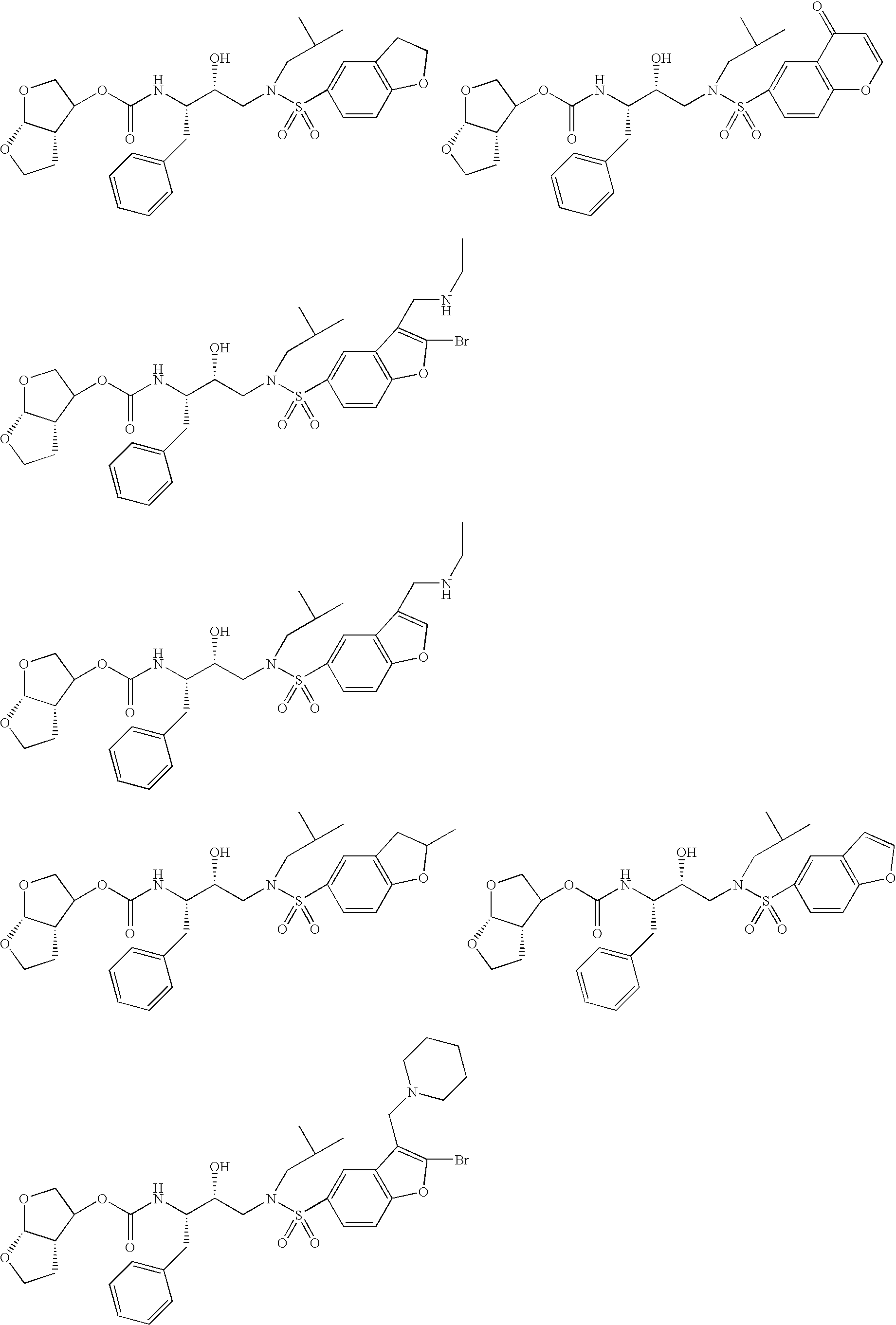 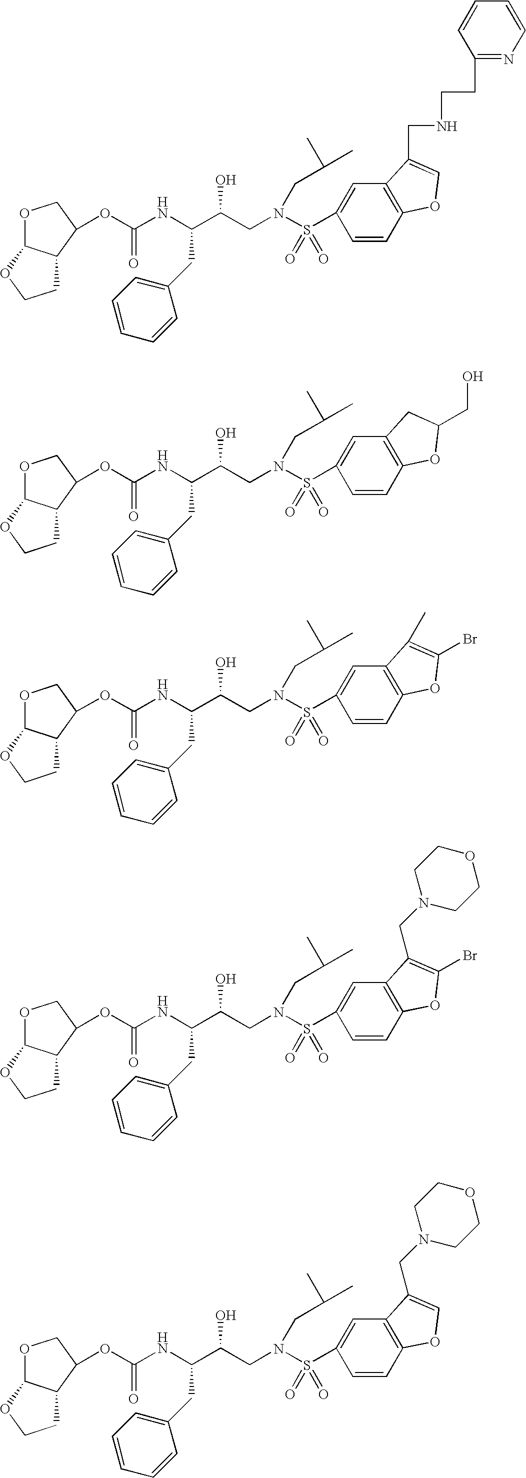 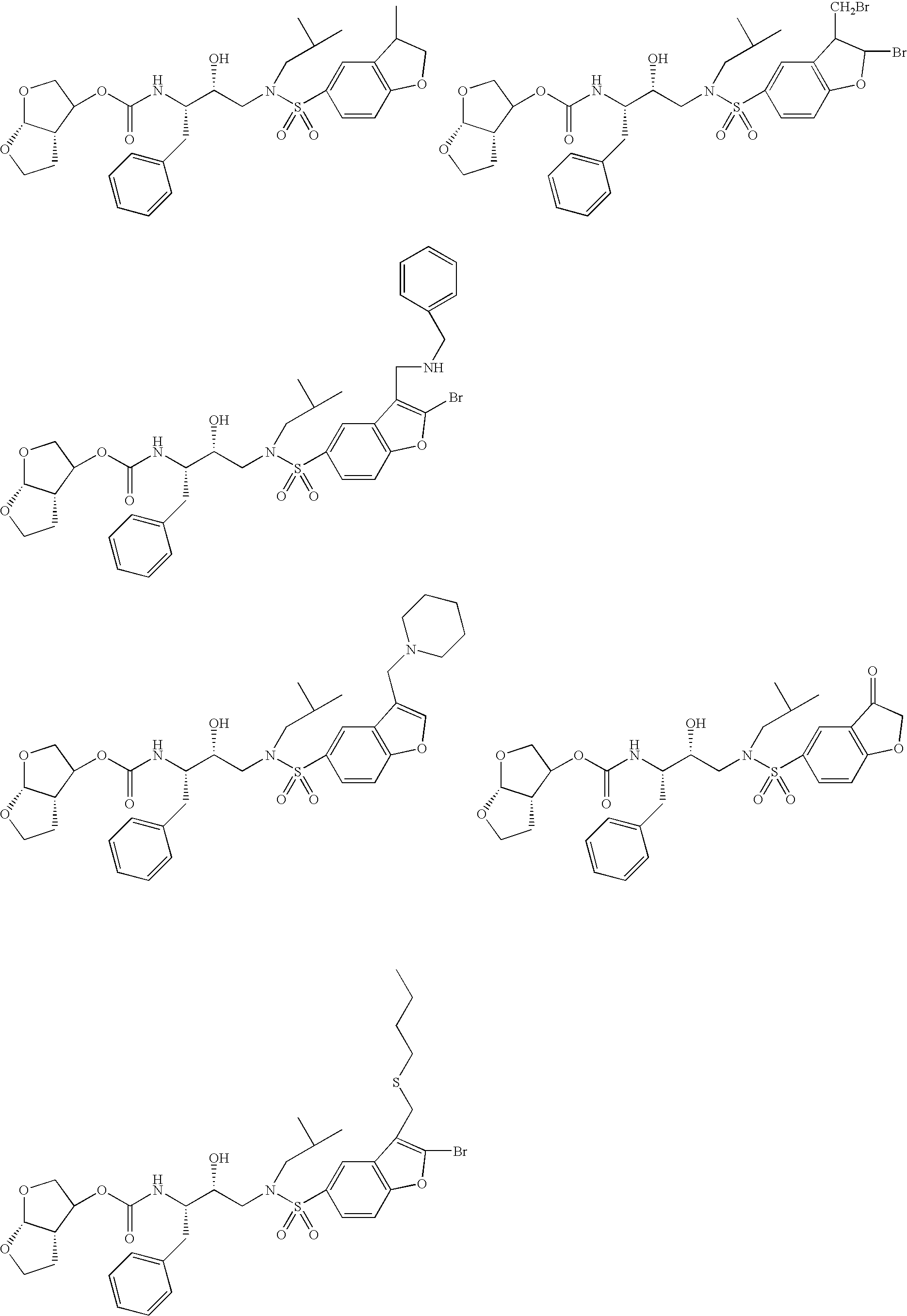 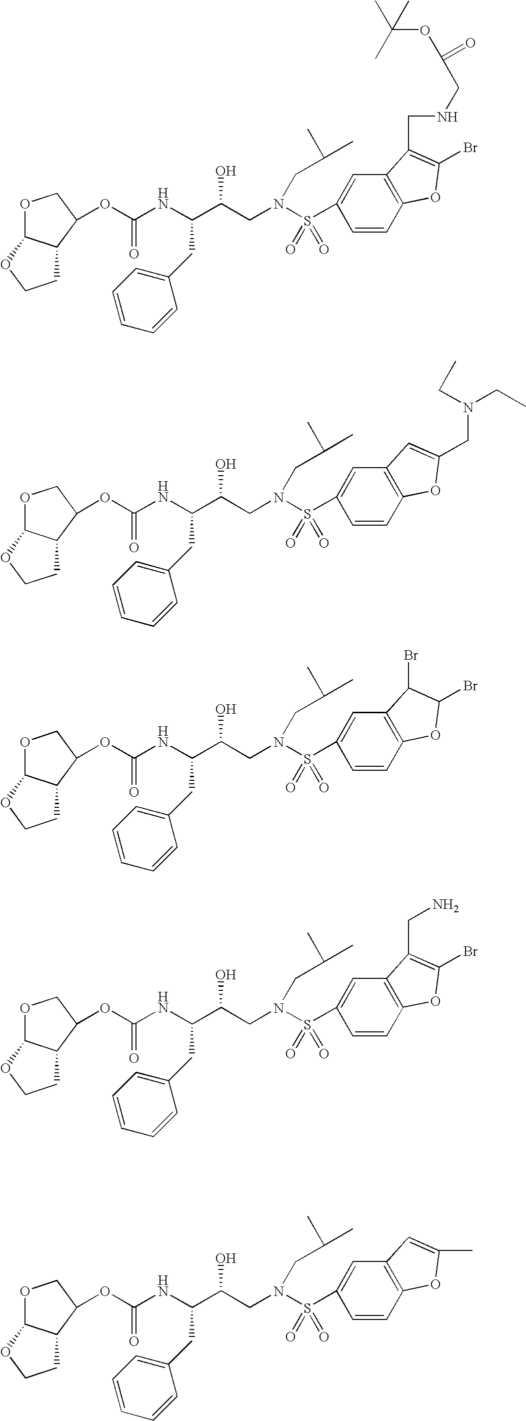 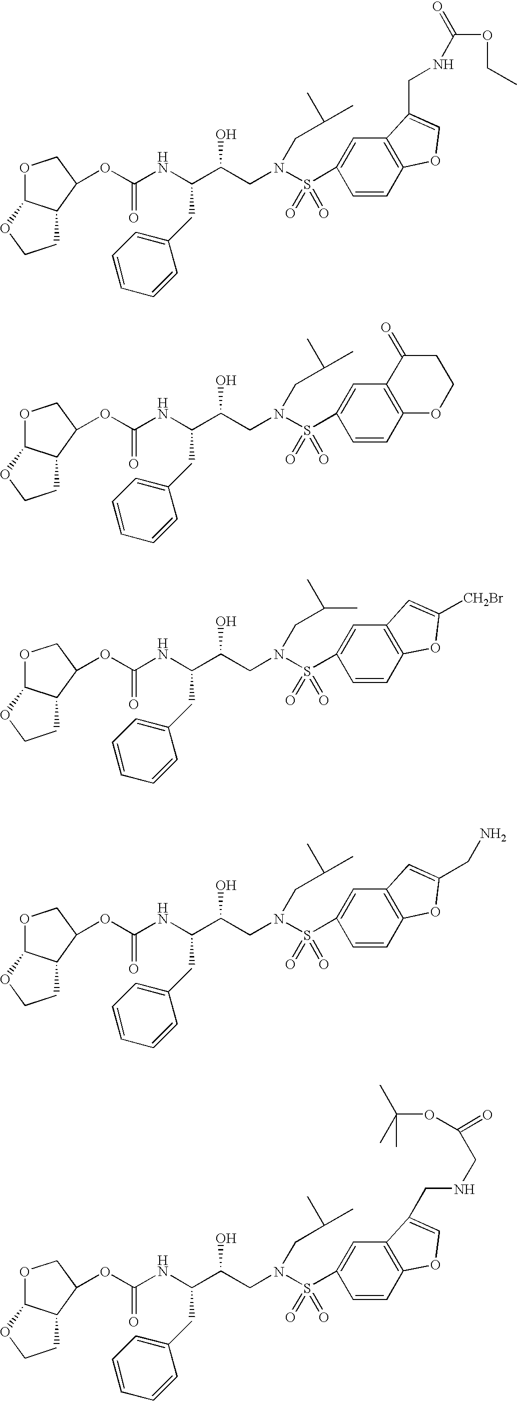 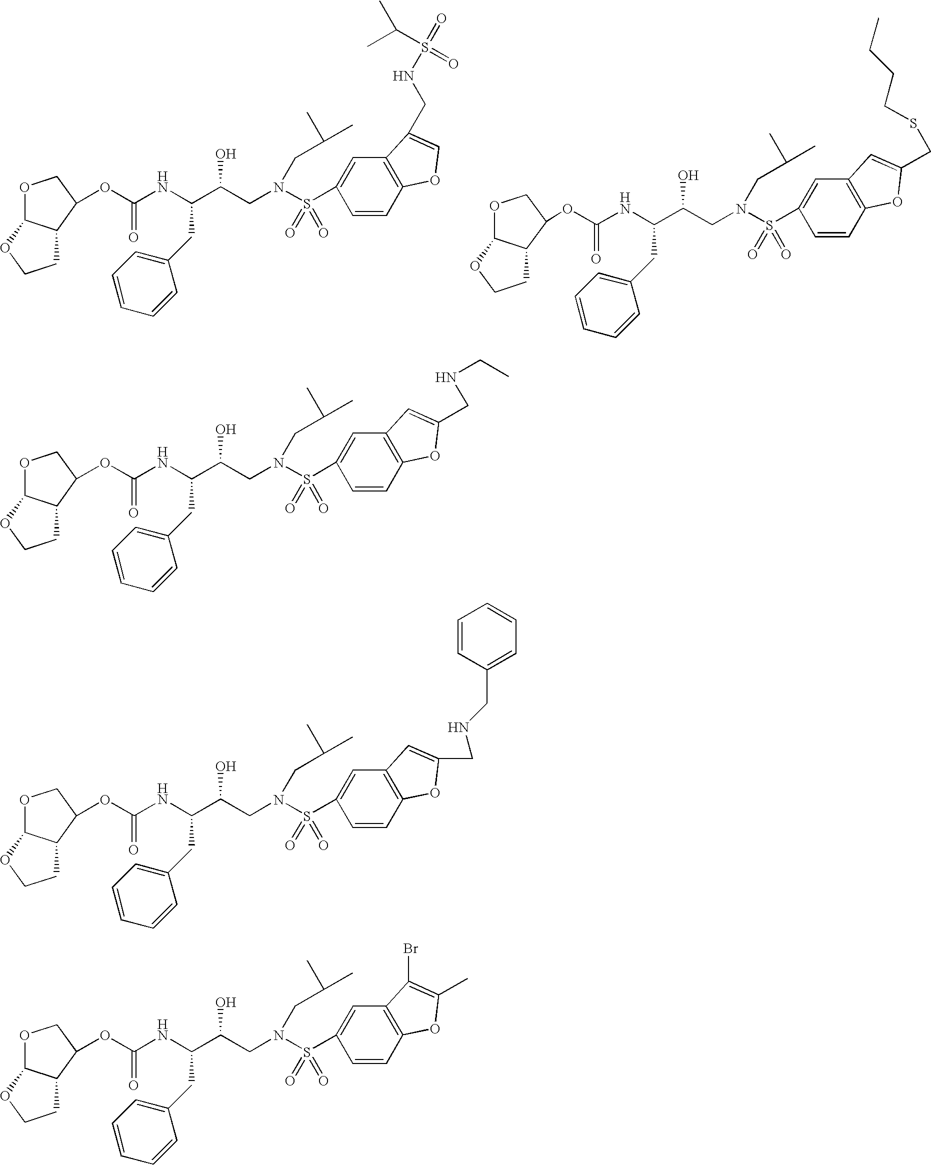 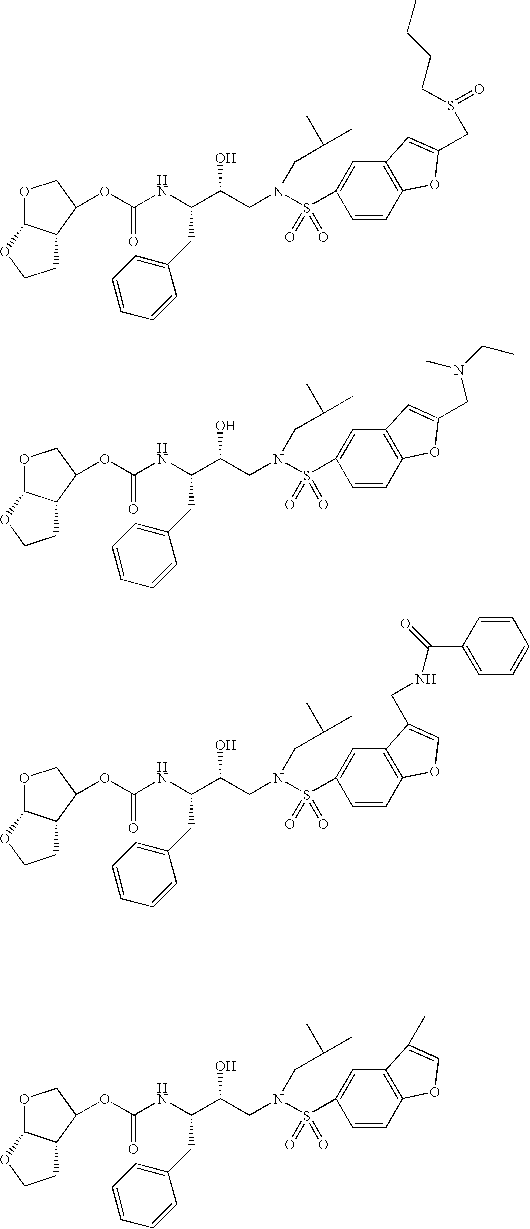 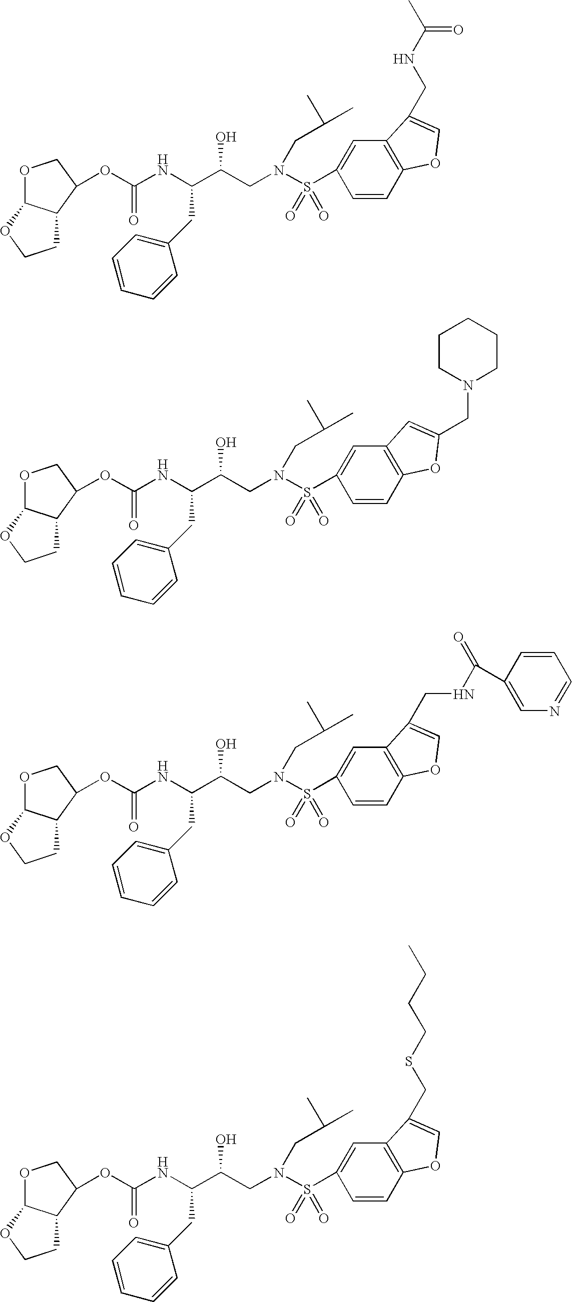 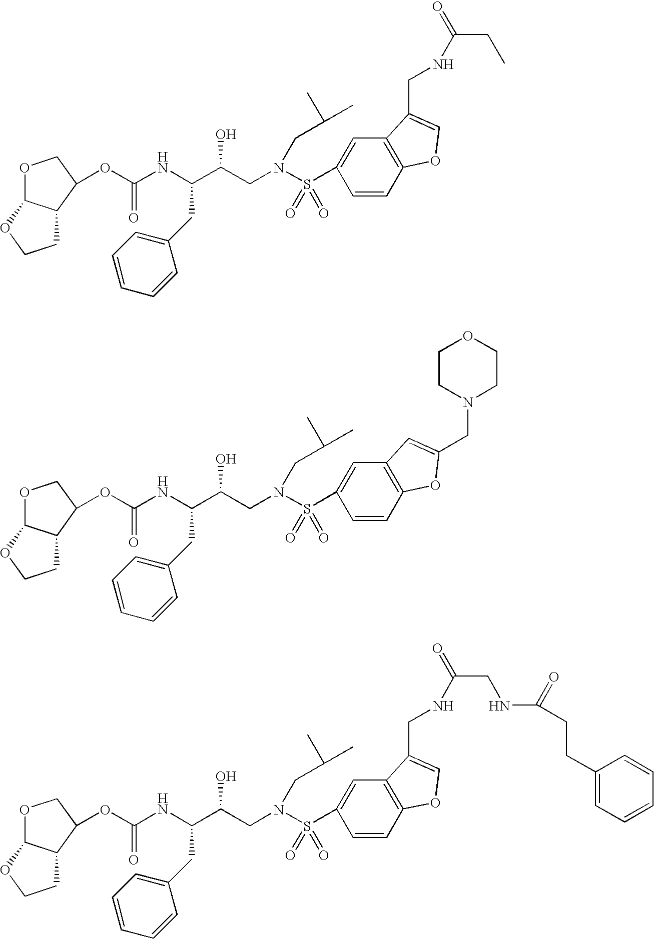 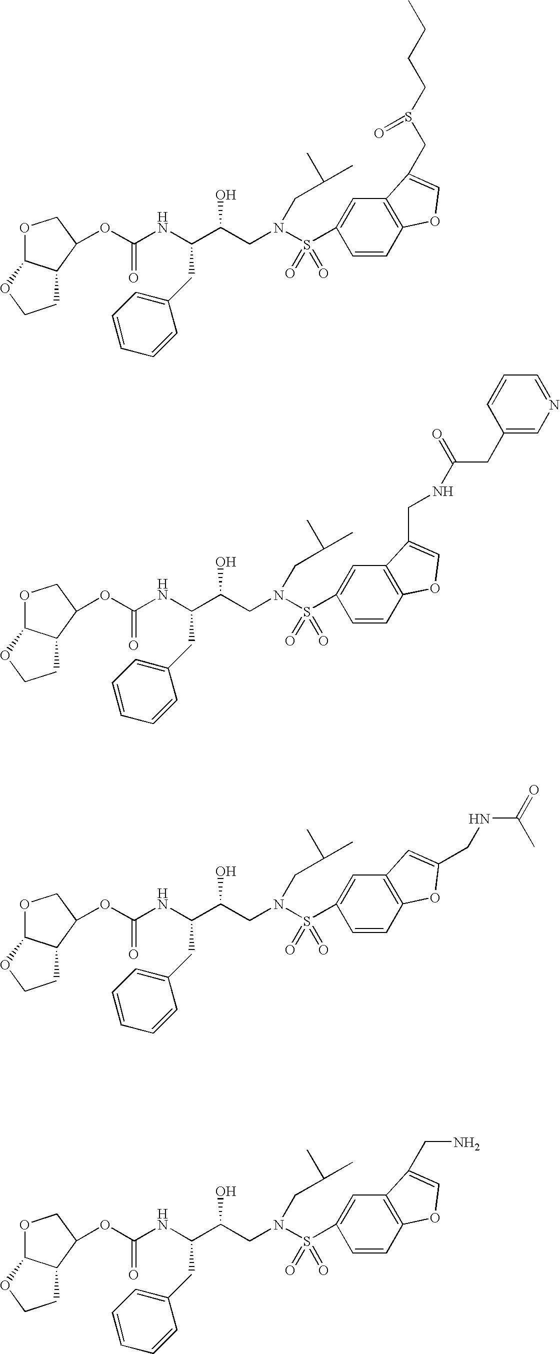 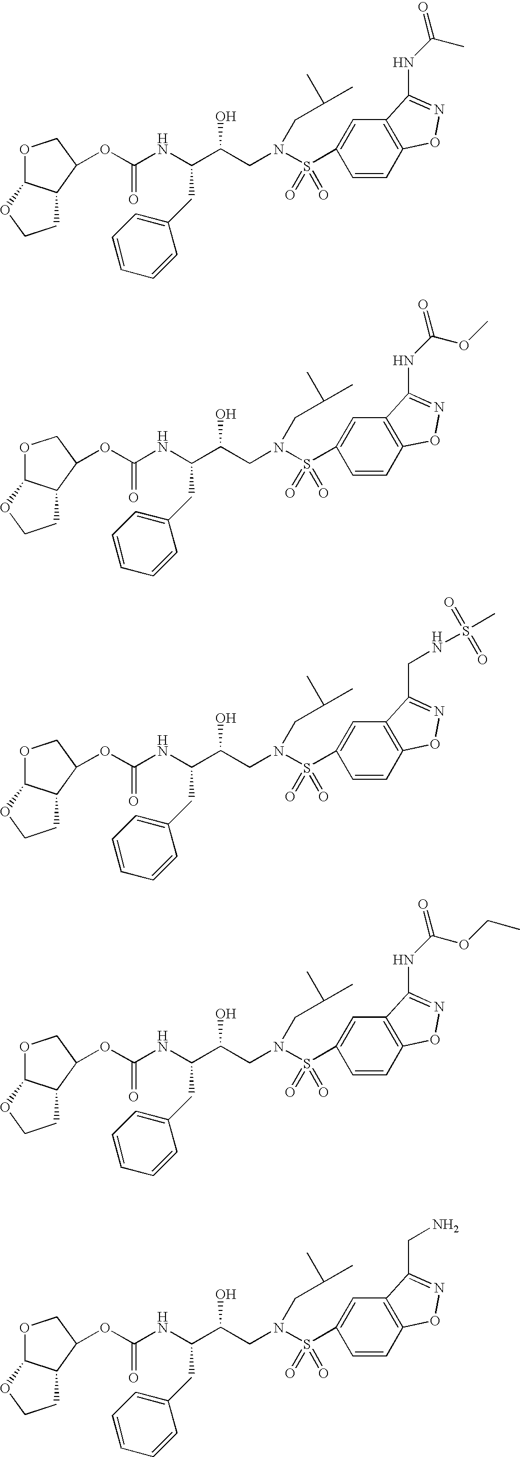 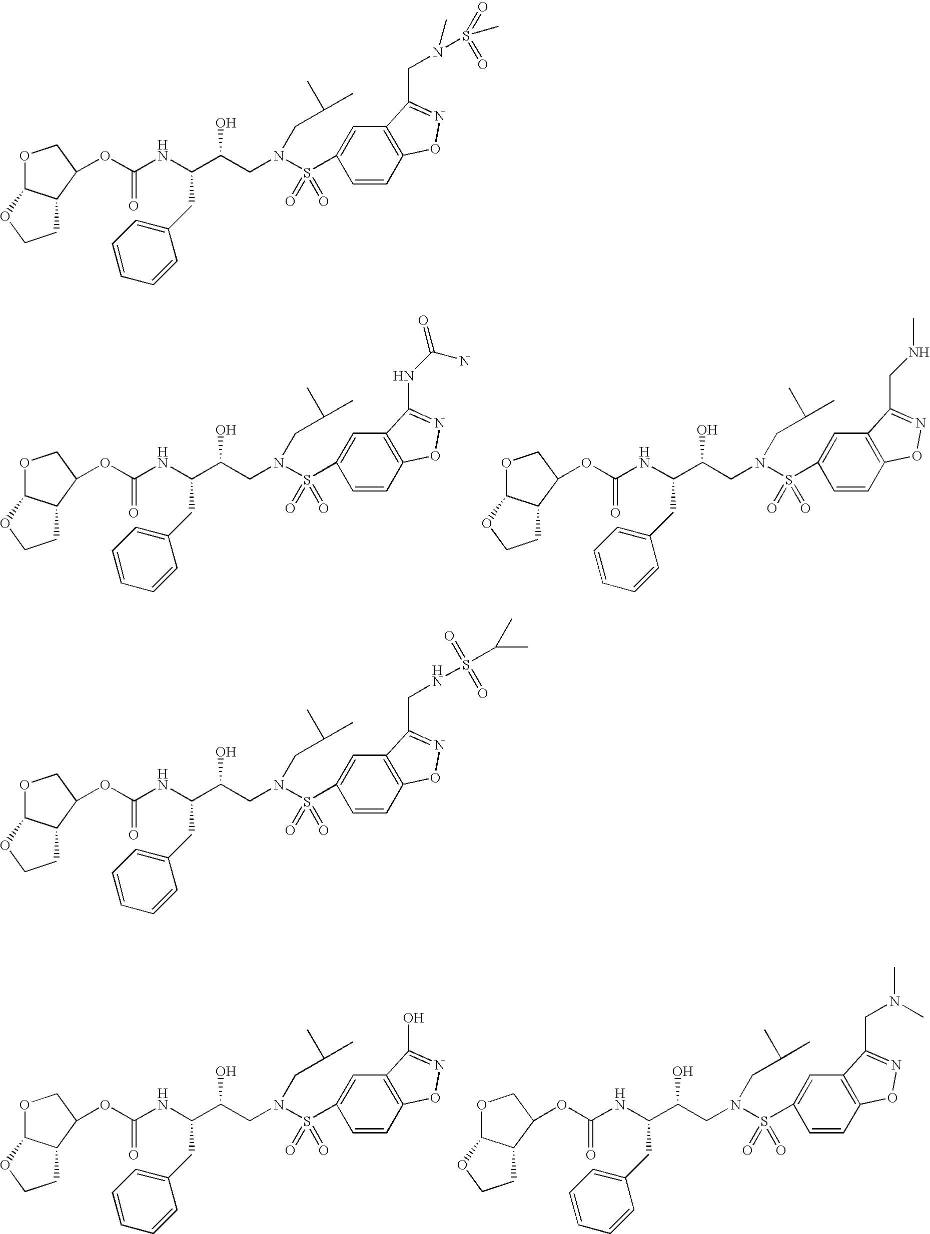 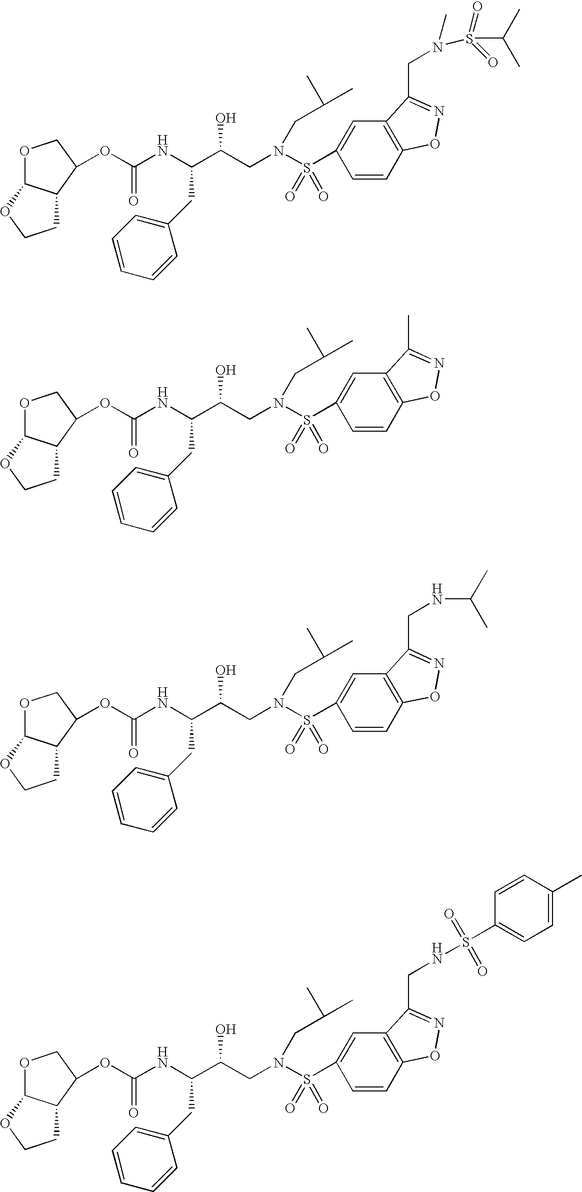 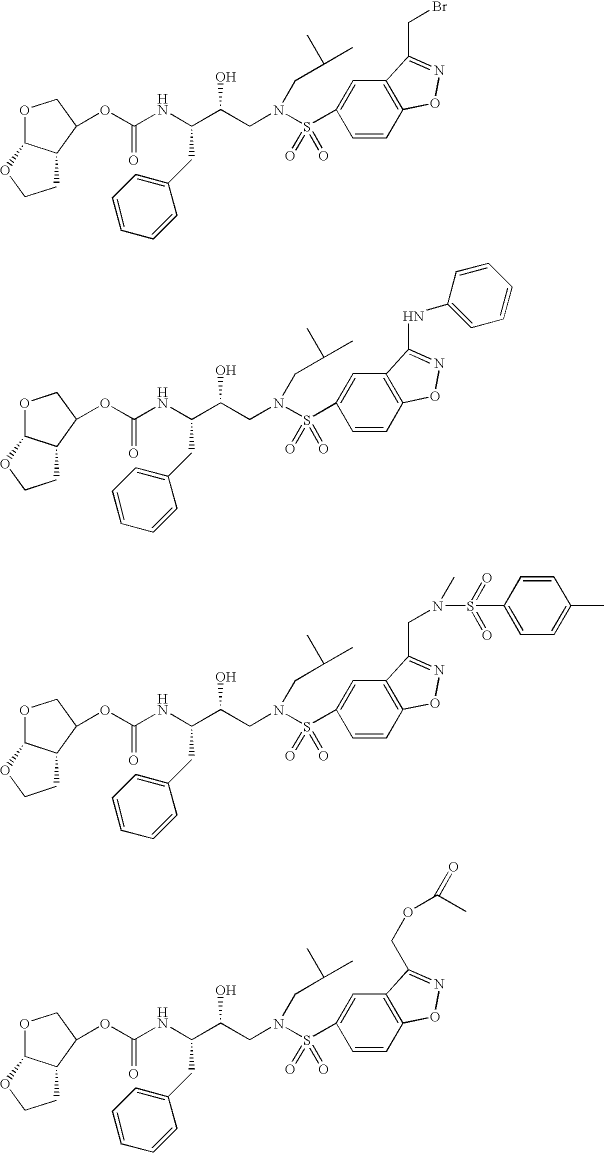 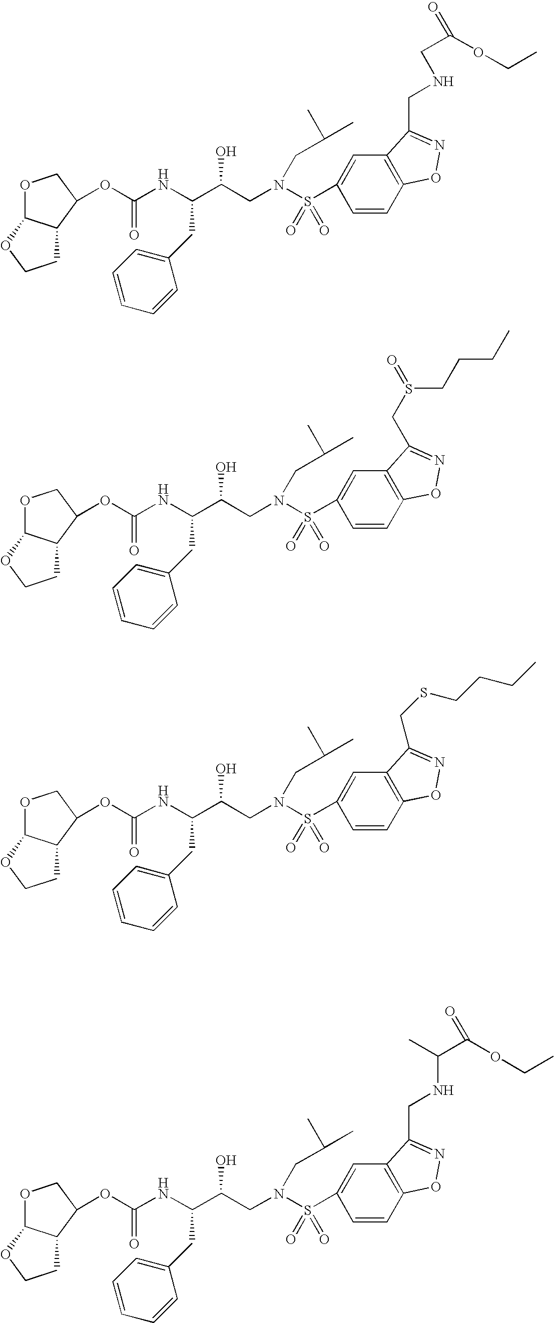 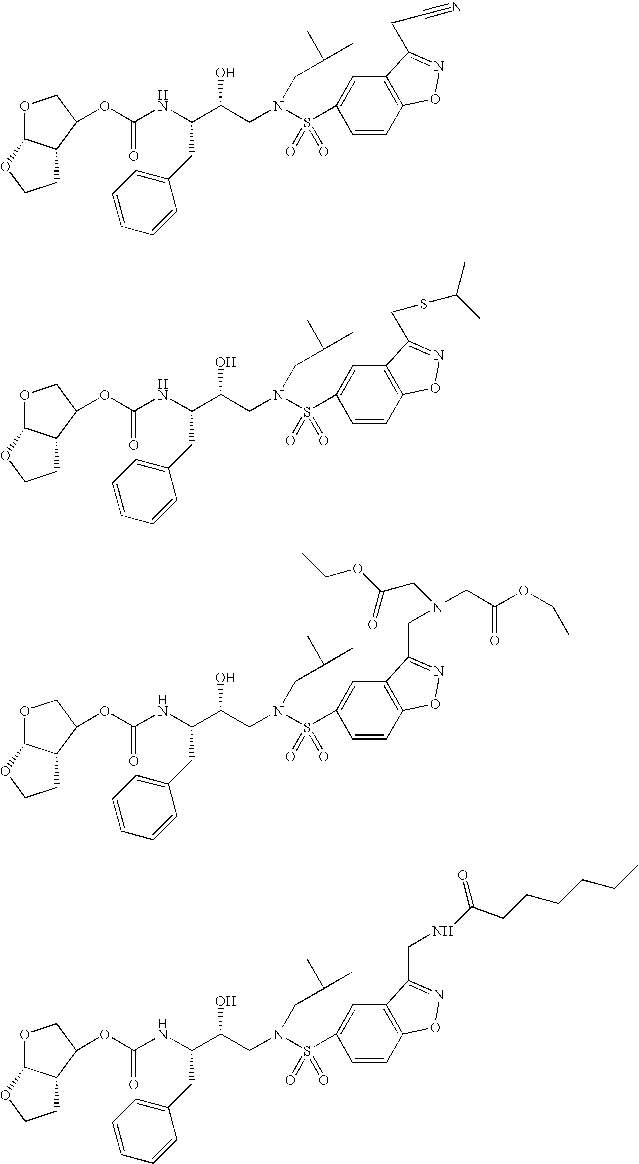 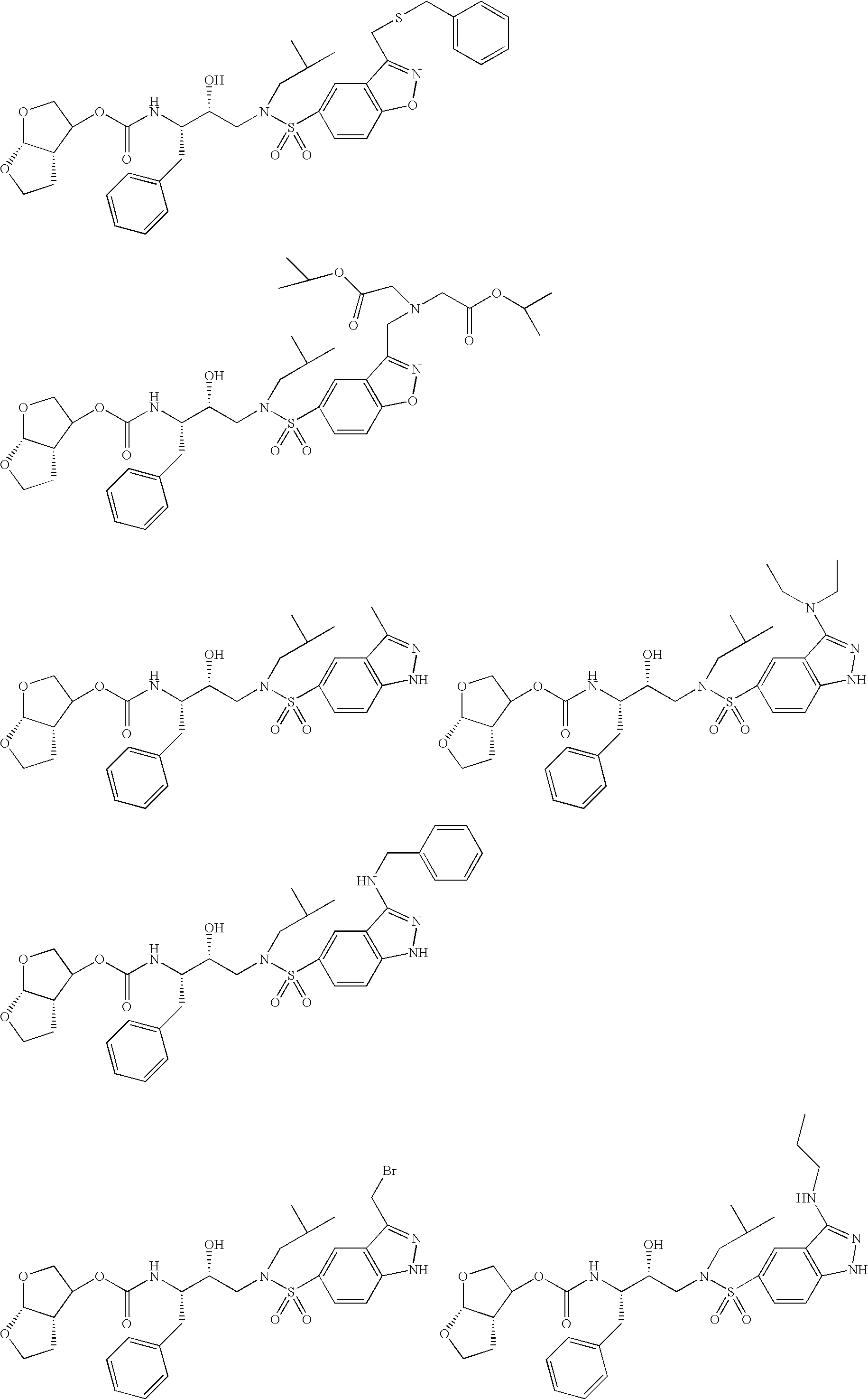 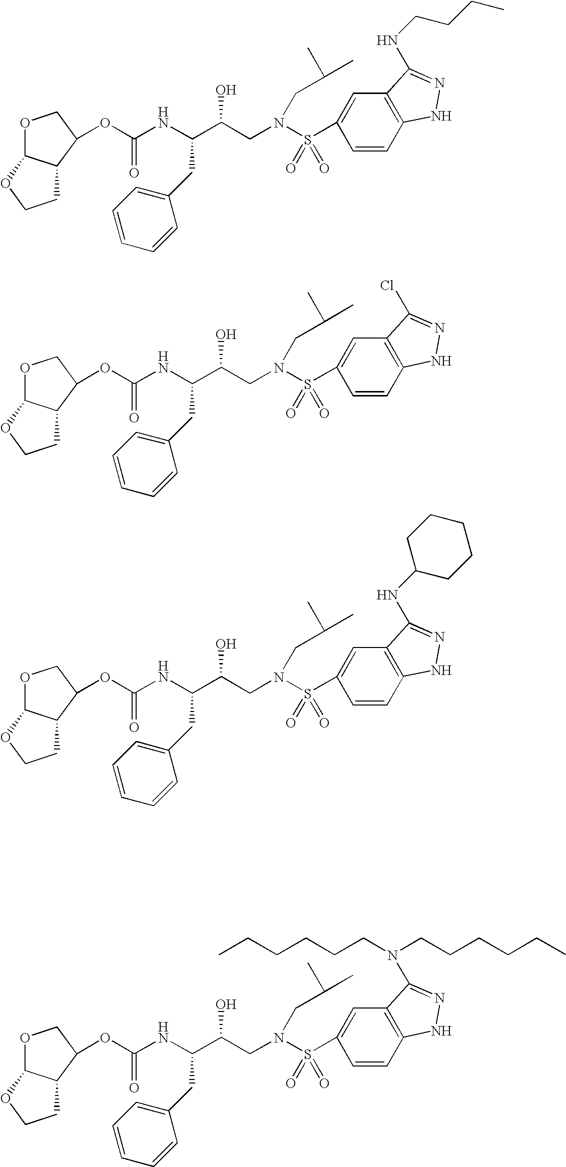 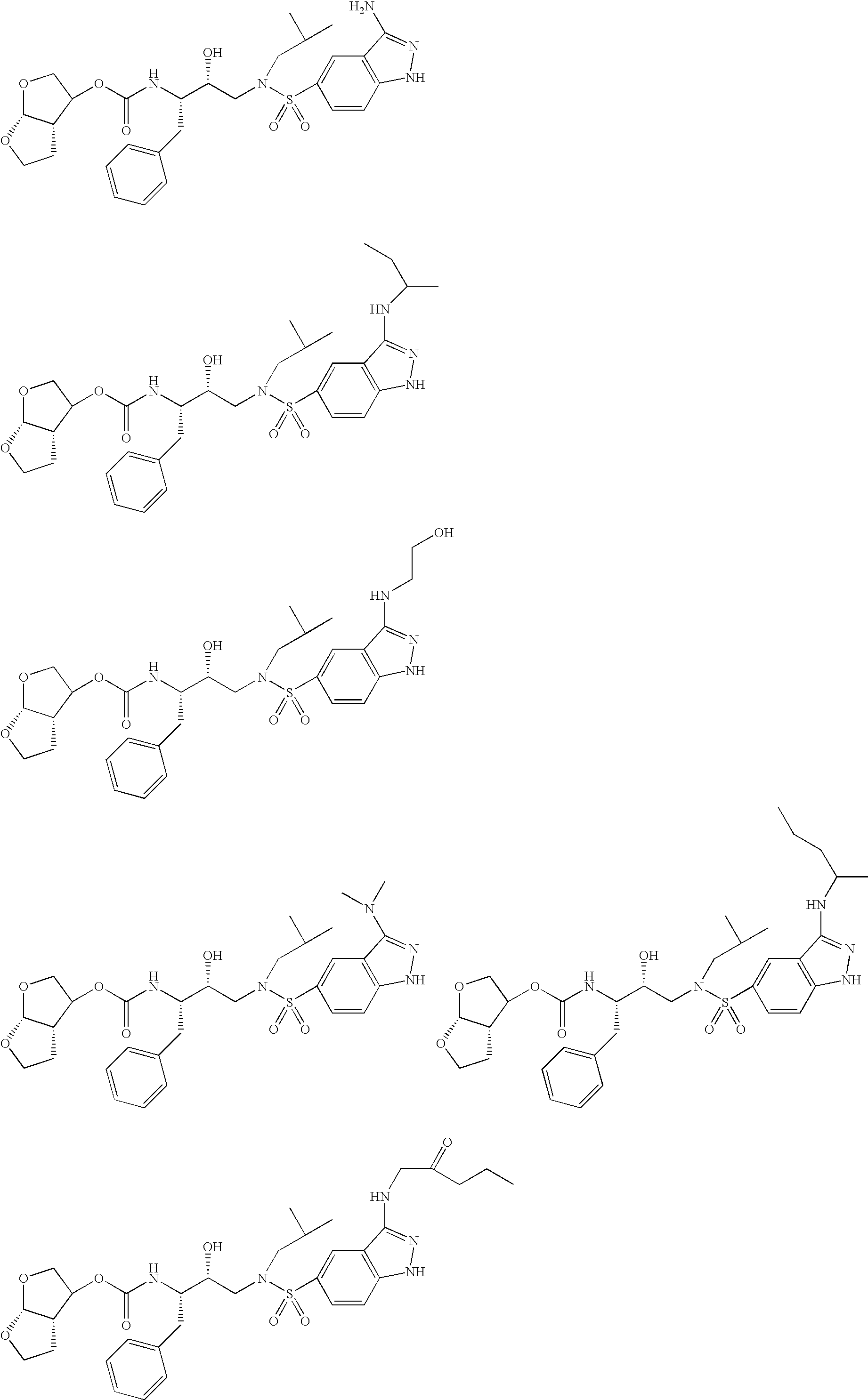 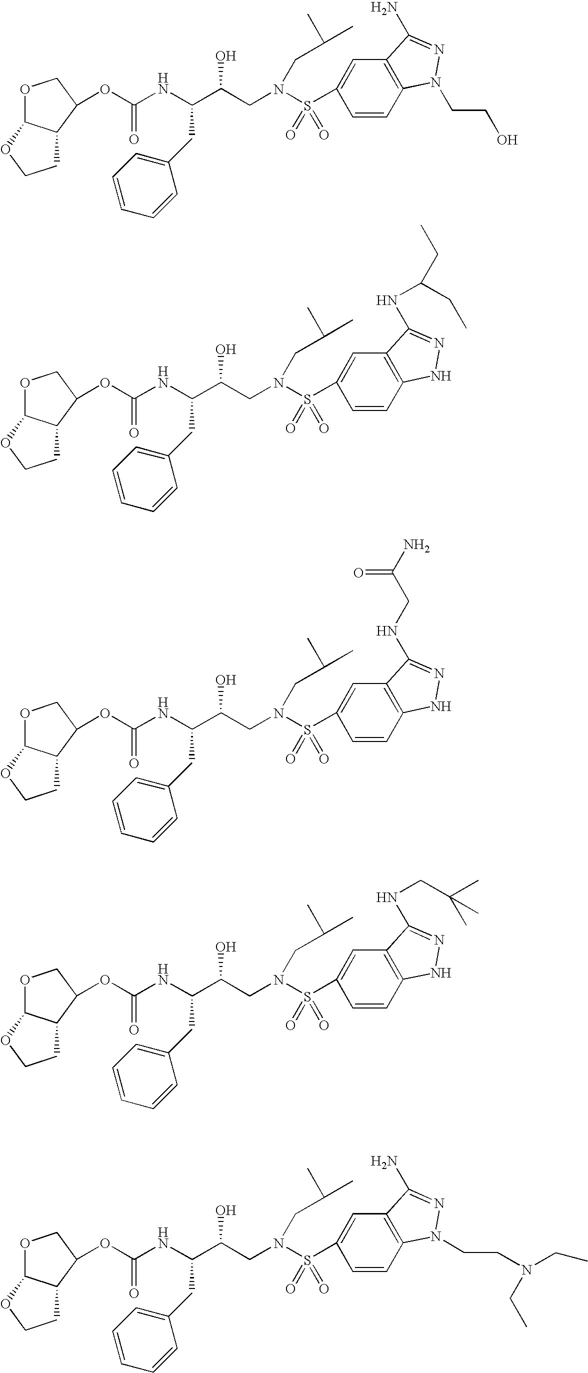
or a pharmaceutically acceptable salt, solvate or ester thereof.

83. A pharmaceutical composition comprising a therapeutically effective amount of the medicament of claim 32, and a pharmaceutically acceptable carrier.

84. A pharmaceutical kit comprising (a) as defined in claim 32, and (b) as defined in claim 32, in separate unit dosage forms, said forms being suitable for administration of (a) and (b) in effective amounts, and instructions for administering (a) and (b).

85. The medicament of claim 32, wherein at least one CYP3A4 inhibitor is:
7H-furo[3,2-g][1]benzopyran-7-one, 4-[[(2E)-5-[(4R)-4′-[[(2E)-3,7-dimethyl-2,6-octadienyl]oxy]-5,5-dimethylspiro[1,3-dioxolane-2,7′-[7H]furo[3,2-g][1]benzopyran]-4-yl]-3-methyl-2-pentenyl]oxy];
7H-furo[3,2-g][1]benzopyran-7-one, 4-[[(2E)-5-[(4R)-4′-[[2E)-6,7-dihydroxy-3,7-dimethyl-2-octenyl]oxy]-5,5-dimethylspiro[1,3-dioxolane-2,7′-[7H]furo[3,2-g][1]benzopyran]-4-yl]-3-methyl-2-pentenyl]oxy]; or
7H-furo[3,2-g][1]benzopyran-7-one, 4-[[(2E)-5-[(2R,4R)-4′-[[(2E,6R)-6,7-dihydroxy-3,7-dimethyl-2-octenyl]oxy]-5,5-dimethylspiro[1,3-dioxolane-2,7′-[7H]furo[3,2-g][1]benzopyran]-4-yl]-3-methyl-2-pentenyl]oxy];
or a pharmaceutically acceptable salt, solvate or ester thereof.

86. The medicament of claim 32, wherein at least one HCV protease inhibitor is administered in an amount ranging from about 100 to about 3600 mg per day.

87. The medicament of claim 32, further comprising at least one aldo-keto reductase (AKR) inhibitor.

88. The medicament of claim 32, further comprising at least one permeability-glycoprotein (Pgp) inhibitor.

89. The medicament of claim 33, wherein at least one other therapeutic agent is an interleukin-10 (IL-10) antagonist or an IL-10 receptor antagonist.
